SEQUENCE LISTING (52 sequences)

SEQ ID NO: 1 - DNA, 1092 bp, Hepatitis C virus
aggataggcttgataggggggacgacatcccttaacaccttcagggttgcgtctatcttctcacctgggccgtcccagaagattcaacttgtgaacaccaacggcagctggcacatcaacaggactgctctggactgcaaagcctccctccagactgggtttcttgctgggctgctctacgcgaaaaagttcaacacgtccggatgcttggagcgtatggccagttgtcgccccattgatacgttcgctcagggatggggtcccatcacctatacagagcgtgtcaactcggaccagaagccttattgctggcactacgcaccacgacggtgtggtatcatacccgcgtcgcaggtgtgcggtccagtctattgtttcaccccaagccccgttgtggtggggacgaccgatcatcttggtgcccctacgtccgtccggggggagaatggaacagacgtgctgctcctcaacaacacgcggccgccacaaggtaactggttcggctgtacatggatgaatagtactgggttcaccaagacctgcggaggccccccctgcaacatcgggggggggggcggcaacaacaccctgacctgccccacggactgcttccgaaagcaccccgaggctacgtacacaaaatgtggttcggggccttggttgacacctaggtgcttggttgactacccatacaggctctggcactacccctgcactttcaattttaccatcttcaaggtcaggatgtatgtggggggtgtggaacacaggctcagcgttgcatgcaactggactcggggagagcgttgtaatctggaggacagggatagatcagagctcagtccgctgctgctgtctacgacagagtggcagatactgccctgttccttcaccaccctgccagctctgtccaccggtttgattcatctccatcagggcatcgtggacgtgcaatacctgtatggtatagggtcagcgtttgtctcctttgcaatcaagtgggagtatgtcctgttgcttttcctcctcctggcggacgcgcgcgtctgtgcctgcttgtggatgatgctgttgatagcccaggctgaggcc

SEQ ID NO: 2 - DNA, 1092 bp, Hepatitis C virus
cggatcggcctgatcggcggcaccacctccctgaacaccttccgggtggccagcatcttcagccctggccccagccagaaaatccagctggtgaacaccaacggcagctggcacatcaaccggaccgccctggactgcaaggccagcctgcagaccggcttcctggccgggctgctgtacgccaagaagttcaacaccagcggctgcctggagcggatggccagctgccggcccatcgacaccttcgcccagggctggggccccatcacctacaccgagagagtgaacagcgaccagaagccctactgctggcactacgcccccagaagatgcggcatcatccccgcctcccaggtgtgcggccctgtgtactgcttcacccccagccccgtggtggtgggcacaaccgaccacctgggagcccccacctccgtgcggggcgagaacggcaccgacgtgctgctgctgaacaacaccagacccccccagggcaattggttcggctgcacctggatgaacagcaccggcttcaccaagacctgcggcggacccccctgcaacatcggcggagggggcggaaacaacaccctgacctgccccaccgactgttttcggaagcaccccgaggccacctacaccaagtgcggctccggcccctggctgaccccccggtgcctggtggactacccctaccggctgtggcactacccctgcaccttcaacttcaccatcttcaaagtgcggatgtatgtgggcggcgtggagcacagactgagcgtggcctgcaactggaccaggggcgagcggtgcaacctggaggaccgggaccggagcgagctgtcccccctgctgctgtccaccaccgagtggcagatcctgccctgcagcttcaccaccctgcccgccctgagcaccggcctgatccacctgcaccagggcatcgtggacgtgcagtacctgtacggcatcggcagcgccttcgtgagcttcgccatcaagtgggagtatgtgctgctgctgttcctgctgctggccgacgccagagtgtgcgcctgcctgtggatgatgctgctgattgcccaggccgaggcc

SEQ ID NO: 3 - protein, 364 aa, Hepatitis C virus
RIGLIGGTTSLNTFRVASIFSPGPSQKIQLVNTNGSWHINRTALDCKASLQTGFLAGLLYAKKFNTSGCLERMASCRPIDTFAQGWGPITYTERVNSDQKPYCWHYAPRRCGIIPASQVCGPVYCFTPSPVVVGTTDHLGAPTSVRGENGTDVLLLNNTRPPQGNWFGCTWMNSTGFTKTCGGPPCNIGGGGGNNTLTCPTDCFRKHPEATYTKCGSGPWLTPRCLVDYPYRLWHYPCTFNFTIFKVRMYVGGVEHRLSVACNWTRGERCNLEDRDRSELSPLLLSTTEWQILPCSFTTLPALSTGLIHLHQGIVDVQYLYGIGSAFVSFAIKWEYVLLLFLLLADARVCACLWMMLLIAQAEA

SEQ ID NO: 4 - DNA, 1089 bp, Hepatitis C virus
gatacccacgtgacagggggggcgcaagccaaaaccaccaacaggctcgtgtccatgttcgcaagtgggccgtctcagaaaatccagcttataaacaccaatggcagttggcacatcaacaggactgccctgaactgcaatgactctctccagactgggtttcttgccgcgctgttctacacacatagtttcaactcgtccgggtgcccagagcgcatggccagctgccgcaccattgacaagttcgaccagggatggggtcccattacttatgctgagtctagcagatcagaccagaggccatattgctggcactacccacctccacaatgtaccatcgtacctgcgtcggaggtgtgcggcccagtgtactgcttcaccccaagccctgtcgtcgtggggacgaccgatcgtttcggtgtccctacgtatagatggggggagaacgagactgacgtgctgctgctcaacaacacgcggccgccgcaaggcaactggttcggctgcacatggatgaatagcaccgggttcaccaagacatgtggggggcccccgtgtaacatcgggggggtcggcaacaacaccctgacctgccccacggactgcttccggaagcaccccgaggctacctacacaaaatgtggttcggggccttggctgacacctaggtgcatggttgactatccatacaggctctggcattacccctgcactgttaactttaccatcttcaaggttaggatgtatgtggggggcgtggagcacaggctcaatgctgcatgcaattggacccgaggagagcgttgtgacttggaggacagggatagggcggagctcagcccgctgctgctgtctacaacagagtggcaggtactgccctgttccttcaccaccctaccagctctgtccactggcttgattcacctccatcagaacatcgtggacgtgcaatacctatacggtatagggtcagcggttgtctcctttgcaatcaaatgggagtatgtcctgttgcttttccttctcctagcggacgcacgtgtctgtgcctgcttgtggatgatgctgctgatagcccaggccgaggcc

SEQ ID NO: 5 - protein, 363 aa, Hepatitis C virus
DTHVTGGAQAKTTNRLVSMFASGPSQKIQLINTNGSWHINRTALNCNDSLQTGFLAALFYTHSFNSSGCPERMASCRTIDKFDQGWGPITYAESSRSDQRPYCWHYPPPQCTIVPASEVCGPVYCFTPSPVVVGTTDRFGVPTYRWGENETDVLLLNNTRPPQGNWFGCTWMNSTGFTKTCGGPPCNIGGVGNNTLTCPTDCFRKHPEATYTKCGSGPWLTPRCMVDYPYRLWHYPCTVNFTIFKVRMYVGGVEHRLNAACNWTRGERCDLEDRDRAELSPLLLSTTEWQVLPCSFTTLPALSTGLIHLHQNIVDVQYLYGIGSAVVSFAIKWEYVLLLFLLLADARVCACLWMMLLIAQAEA

SEQ ID NO: 6 - DNA, 1089 bp, Hepatitis C virus
gaaacccacgtcaccgggggaaatgccggccgcaccacggctgggcttgttggtctccttacaccaggcgccaagcagaacatccaactgatcaacaccaacggcagttggcacatcaatagcacggccttgaattgcaatgaaagccttaacaccggctggttagcagggctcttctatcaacacaaattcaactcttcaggctgtcctgagaggttggccagctgccgacgccttaccgattttgcccagggctggggtcctatcagttatgccaacggaagcggcctcgacgaacgcccctactgctggcactaccctccaagaccttgtggcattgtgcccgcaaagagcgtgtgtggcccggtatattgcttcactcccagccccgtggtggtgggaacgaccgacaggtcgggcgcgcctacctacagctggggtgcaaatgatacggatgtcttcgtccttaacaacaccaggccaccgctgggcaattggttcggttgtacctggatgaactcaactggattcaccaaagtgtgcggagcgcccccttgtgtcatcggaggggtgggcaacaacaccttgctctgccccactgattgcttccgcaaacatccggaagccacatactctcggtgcggctccggtccctggattacacccaggtgcatggtcgactacccgtataggctttggcactatccttgtaccatcaattacaccatattcaaagtcaggatgtacgtgggaggggtcgagcacaggctggaagcggcctgcaactggacgcggggcgaacgctgtgatctggaagacagggacaggtccgagctcagcccgttgctgctgtccaccacacagtggcaggtccttccgtgttctttcacgaccctgccagccttgtccaccggcctcatccacctccaccagaacattgtggacgtgcagtacttgtacggggtagggtcaagcatcgcgtcctgggccattaagtgggagtacgtcgttctcctgttccttctgcttgcagacgcgcgcgtctgctcctgcttgtggatgatgttactcatatcccaagcggaggcg

SEQ ID NO: 7 - protein, 363 aa, Hepatitis C virus
ETHVTGGNAGRTTAGLVGLLTPGAKQNIQLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMYVGGVEHRLEAACNWTRGERCDLEDRDRSELSPLLLSTTQWQVLPCSFTTLPALSTGLIHLHQNIVDVQYLYGVGSSIASWAIKWEYVVLLFLLLADARVCSCLWMMLLISQAEA

SEQ ID NO: 8 - DNA, 1092 bp, Hepatitis C virus
cacaccctcacaacgggggggcacgctgcccgcctcaccagcgggttcgcgggcctctttacacctgggccgtctcagagaatccagcttataaacaccaatggcagttggcacatcaacaggactgccctgaactgcaatgactccctccagactgggtttcttgccgcgctgttctacgcacataggttcaactcgtccggatgcccggggcgcatggccagctgccgctccattgacaagttcgaccagggatggggtcctatcacttatgctgagcctacaaaagacccggaccagaggccttattgctggcactacccacctcaacaatgtggtatcgtacctgcgtcgcaggtgtgtggtccagtgtattgcttcaccccaagtcctgttgtcgtggggacaaccgatcgtctcggcaaccctacgtacagctggggggagaacgatactgacgtgctgctccttaacaacacgcggccgccgcaaggcaactggttcggctgtacatggatgaatagcactgggttcaccaagacgtgcggggcccccccgtgtaacatcgggggggtcggcaataacaccttgacctgccccacggactgcttccggaagcaccccgaggccacgtactcaaaatgtggctcggggccttggttgacacctaggtgcatggttgactacccatacaggctctggcactacccctgcactgtcaacttctccatctttaaggttaggatgtatgtggggggcgtggagcacaggcttaatgctgcatgcaactggacccgaggagagcgttgcaacttggacgacagggacagatcggagctcagcccgctgctgctctctacaacagagtggcaggttctgccctgctctttcaccaccctaccggctctgtccactggcttgatccacctccatcagaacatcgtggacgtgcaatacctgtacggtatagggtcagcggttgtctcctttgcaatcaaatgggagtatgtcgtgttgcttttccttctcctggcggacgcgcgcgtctgtgcctgcttgtggatgatgctgctgatagcccaggccgaggcc

SEQ ID NO: 9 - protein, 364 aa, Hepatitis C virus
HTLTTGGHAARLTSGFAGLFTPGPSQRIQLINTNGSWHINRTALNCNDSLQTGFLAALFYAHRFNSSGCPGRMASCRSIDKFDQGWGPITYAEPTKDPDQRPYCWHYPPQQCGIVPASQVCGPVYCFTPSPVVVGTTDRLGNPTYSWGENDTDVLLLNNTRPPQGNWFGCTWMNSTGFTKTCGAPPCNIGGVGNNTLTCPTDCFRKHPEATYSKCGSGPWLTPRCMVDYPYRLWHYPCTVNFSIFKVRMYVGGVEHRLNAACNWTRGERCNLDDRDRSELSPLLLSTTEWQVLPCSFTTLPALSTGLIHLHQNIVDVQYLYGIGSAVVSFAIKWEYVVLLFLLLADARVCACLWMMLLIAQAEA

SEQ ID NO: 10 - RNA, 39 nt, artificial
  synthetic polyadenylation signal
cuuuauuuucauuagaucuguguguugguuuuuugugug

SEQ ID NO: 11 - protein, 14 aa, Hepatitis C virus
KVLIVMLLFAGVDG

SEQ ID NO: 12 - protein, 14 aa, Hepatitis C virus
KVLIVMLLFAGVDG

SEQ ID NO: 13 - protein, 14 aa, Hepatitis C virus
KVLVVLLLFAGVDA

SEQ ID NO: 14 - DNA, 69 bp, Artificial
  tissue plasminogen activator signal sequence
atggatgcaatgaagagagggctctgctgtgtgctgctgctgtgtggagcagtcttcgtttcgcccagc

SEQ ID NO: 15 - protein, 23 aa, Artificial
  tissue plasminogen activator signal sequence
MDAMKRGLCCVLLLCGAVFVSPS

SEQ ID NO: 16 - DNA, 576 bp, Hepatitis C virus
tatgaagtgcgcaacgtgtccgggggataccatgtcacgaacgactgctccaactcaagcattgtgtacgagacagcggacatgatcatgcataccccggggtgcgtgccctgcgttcgggagagcaattactcccgttgctgggtagcgcttactcccacgcttgcggccaggaataacagcgtccctaccatgacaatacgacgccacatcgatttgctcgtggggacggctgctttctgctccgctatgtacgtgggggatctctgcggatctgtcttcctcgtctcccagctgttcaccttctcgcctcgccggcatgagacggtacaggactgcaattgttcaatctatcccggccatctaacaggtcaccgcatggcttgggatatgatgatgaattggtcacctacagcagccctagtggtgtcgcagttactccggatcccacaagctgtcgtggacatggtggcgggggcccactggggggtcctggcgggccttgcctactattccatggtggggaactgggctaaggtgttgatcgtgatgctgctcttcgccggcgttgacggg

SEQ ID NO: 17 - protein, 192 aa, Hepatitis C virus
YEVRNVSGGYHVTNDCSNSSIVYETADMIMHTPGCVPCVRESNYSRCWVALTPTLAARNNSVPTMTIRRHIDLLVGTAAFCSAMYVGDLCGSVFLVSQLFTFSPRRHETVQDCNCSIYPGHLTGHRMAWDMMMNWSPTAALVVSQLLRIPQAVVDMVAGAHWGVLAGLAYYSMVGNWAKVLIVMLLFAGVDG

SEQ ID NO: 18 - DNA, 576 bp, Hepatitis C virus
tacgaagtgcacaacgtgtccgggatatatcatgtcacgaacgactgctccaacgcaagcattgtgtatgaggcagcggacttgatcatgcatactcctgggtgcgtgccctgcgttcgggaaggcaactcctcccgctgctgggtagcgctcactcccacgctcgcagccaggaacgtcaccatccccaccacgacgatacgacgccacgtcgatctgctcgttggggcggctgctttctgttccgctatgtacgtgggggacctctgcggatctgttttcctcgtctctcagctgttcaccttctcgcctcgccggcatgtgacattacaggactgtaactgctcaatttatcccggccatgtgtcgggtcaccgtatggcttgggacatgatgatgaactggtcgcccacaacagccctagtggtgtcgcagttactccggatcccacaagccgtcgtggacatggtggcgggggcccactggggagtcctggcgggccttgcctactattccatggcggggaactgggctaaggttctgattgtgatgctactttttgctggcgttgacggg

SEQ ID NO: 19 - protein, 192 aa, Hepatitis C virus
YEVHNVSGIYHVTNDCSNASIVYEAADLIMHTPGCVPCVREGNSSRCWVALTPTLAARNVTIPTTTIRRHVDLLVGAAAFCSAMYVGDLCGSVFLVSQLFTFSPRRHVTLQDCNCSIYPGHVSGHRMAWDMMMNWSPTTALVVSQLLRIPQAVVDMVAGAHWGVLAGLAYYSMAGNWAKVLIVMLLFAGVDG

SEQ ID NO: 20 - DNA, 576 bp, Hepatitis C virus
taccaagtgcgcaattcctcggggctttaccatgtcaccaatgattgccctaactcgagtattgtgtacgaggcggccgatgccatcctgcacactccggggtgtgtcccttgcgttcgcgagggtaacgcctcgaggtgttgggtggcggtgacccccacggtggccaccagggacggcaaactccccacaacgcagcttcgacgtcatatcgatctgcttgtcgggagcgccaccctctgctcggccctctacgtgggggacctgtgcgggtctgtctttcttgttggtcaactgtttaccttctctcccaggcgccactggacgacgcaagactgcaattgttctatctatcccggccatataacgggtcatcgcatggcatgggatatgatgatgaactggtcccctacggcagcgttggtggtagctcagctgctccggatcccacaagccatcatggacatgatcgctggtgctcactggggagtcctggcgggcatagcgtatttctccatggtggggaactgggcgaaggtcctggtagtgctgctgctatttgccggcgtcgacgcg

SEQ ID NO: 21 - protein, 192 aa, Hepatitis C virus
YQVRNSSGLYHVTNDCPNSSIVYEAADAILHTPGCVPCVREGNASRCWVAVTPTVATRDGKLPTTQLRRHIDLLVGSATLCSALYVGDLCGSVFLVGQLFTFSPRRHWTTQDCNCSIYPGHITGHRMAWDMMMNWSPTAALVVAQLLRIPQAIMDMIAGAHWGVLAGIAYFSMVGNWAKVLVVLLLFAGVDA

SEQ ID NO: 22 - DNA, 576 bp, Hepatitis C virus
catgaagtgcgtaacgcgtccggggtataccatgtcacgaacgactgctccaactcaagcattgtgtttgaggcggcggacttgatcatgcatactcccgggtgcgtgccctgcgttcgggagggtaactcctcccgctgctgggtagcgctcactcccacgctcgcggccaggaatgctaccatccccactacgacaatacgacaccacgtcgatttgctcgttggggcggctgctctctgctccgctatgtacgtgggggacctctgcggatctgttttcctcgtctctcagctgttcaccttctcgccccgccggcatgcgacattgcaggactgcaattgttcgatctaccccggccacgcgtcaggtcaccgcatggcctgggacatgatgatgaactggtcacctacaacagccctcgtagtgtcgcagttactccggatcccacaagccgtcatcgacatggtggcgggggcccactggggagtcctggcgggccttgcctactattccatggcggggaactgggctaaggttttgattgtgatgctactttttgccggcgttgacggg

SEQ ID NO: 23 - protein, 192 aa, Hepatitis C virus
HEVRNASGVYHVTNDCSNSSIVFEAADLIMHTPGCVPCVREGNSSRCWVALTPTLAARNATIPTTTIRHHVDLLVGAAALCSAMYVGDLCGSVFLVSQLFTFSPRRHATLQDCNCSIYPGHASGHRMAWDMMMNWSPTTALVVSQLLRIPQAVIDMVAGAHWGVLAGLAYYSMAGNWAKVLIVMLLFAGVDG

SEQ ID NO: 24 - DNA, 23 bp, Artificial
  primer HCV JFH1 Taqman Probe, 5′ 6FAM labeled, 3′ TAMRA labeled
aaaggacccagtcttcccggcaa

SEQ ID NO: 25 - DNA, 19 bp, Artificial
  primer HCV-JFH1-S147
tctgcggaaccggtgagta

SEQ ID NO: 26 - DNA, 22 bp, Artificial
  primer HCV JFH1-A221
gggcatagagtgggtttatcca

SEQ ID NO: 27 - DNA, 20 bp, Artificial
  TLR-9 agonist
tccatgacgttcctgacgtt

SEQ ID NO: 28 - DNA, 37772 bp, Adenoviridae - Mastadenovirus
catcatcaataatataccttattttggattgaagccaatatgataatgaggtgggcggagcggggcggggcggggaggagcggcggcgcggggcgggccgggaggtgtggcggaagttgagtttgtaagtgtggcggatgtgacttgctagcgccggatgtggtaaaagtgacgttttttggagtgcgacaacgcccacgggaagtgacatttttcccgcggtttttaccggatgtcgtagtgaatttgggcgttaccaagtaagatttggccattttcgcgggaaaactgaaatggggaagtgaaatctgattaatttcgcgttagtcataccgcgtaatatttgccgagggccgagggactttgaccgattacgtggaggaatcgcccaggtgttttttgaggtgaatttccgcgttccgggtcaaagtctccgttttattattatagtcagctgacgcggagtgtatttatacccgctgatctcgtcaagaggccactcttgagtgccagcgagtagagttttctcctctgccgctccgctctgacaccgggggaaaaatgagacatttcacctacgatggcggtgtcctcaccggccagctggctgcctcggtcctggacgccctgatcgaggaggtattggccgacaattatcctcctccagctcattttgagccacctactcttcacgaactgtatgatttggacgtggtggcacctagcgacccgaacgagcaggcggtttccagtttttttcctgactctatgctgttggccagccaggagggggtcgagctcgagacccctcctccaatcgccgtttctcctgagcctccgaccctgaccaggcagcccgatcgccgtgttggacctgcgactatgccccatctgctgcccgaggtgatcgatctcacctgtaacgagtctggttttccacccagcgaggatgaggacgaagagggtgagcagtttgtgttagattctgtggaggaacccgggcgcggttgcagatcttgtcaataccatcggaaaaatacaggagacccccaaattatgtgttccctgtgttatatgaagacgacctgtatgtttatttacagtaagtttgtgattggtgggtcggtgggctgtagtgtgggtaggtggtctgtggtttttttttttttaatatcagcttgggctaaaaaactgctatggtaatttttttaaggtccggtgtctgaacctgagcaggaagctgaaccggagcctgagagtcgccccaggagaaggcctgcaattctaactagaccgagtgcacctgtagcgagggacctcagcagtgcagagaccaccgattccggtccttcctcatcccctccagagattcatcccgtggtgcctttgtgtcccctcaagcccgttgccgtgagagttagtgggcggagggccgccgtggagagcattgaggacttgcttaatgagacacaggaacctttggacttgagctgtaaacgccctaggcaataaacctgcttacctggactgaatgagttgacgcctatgtttgcttttgaatgacttaatgtgtatataataaagagtgagataatgtttaattgcatggtgtgtttgattggggcggggtttgttgggtatataagcttccctgggctaaacttggttacacttgacctcatggaggcctgggagtgtttagagagctttgccgaagtgcgtgccttgctggaagagagctctaataatacctctgggtggtggaggtatttttggggctctccccaggctaagttagtttgtagaatcaaggaggattacaagtgggaatttgaacagcttttgaaatcctgtggtgagctcttggattctttgaatctgggccaccaggctcttttccaggacaagatcatcaggactttggatttttccacaccggggcgcattgctgccggggttgcttttctagcttttttgaaggataaatggagcgaagagacccacttgagttcgggatacgtcctggattttctggccatacaactgtggagagcatggatcaggcacaagaacagaatgcaactgttgtcttccgtccgtccgttgctgattcagccggaggagcagcagaccgggccggaggaccgggctcgtctggaaccagaagagagggcaccggagaggagcgcgtggaacctgggagccggcctgaacggccatccacatcgggagtgaatgttggacaggtggcggatctctttccagaactgcgacgaatcttaactatcagggaggatggacaatttgttaaggggcttaagagggagcggggggcttctgaacataacgaggaggccagtaatttagcttttagtctgatgaccagacaccgtcccgagtgcattacttttcagcagattaaggataattgtgccaatgagttagatctgctgggtcagaagtacagcatagagcagttgaccacttactggctgcagccgggtgatgatctggaggaagctattagggtgtatgccaaggtggccctgaggcccgattgcaagtacaagctcaaggggctggtgaatatcaggaattgttgctacatttctgggaacggggcggaggtggagatagagaccgatgacagggtggcctttaggtgtagcatgatgaatatgtggcctggggtgctgggcatggacggggtggtgattatgaatgtgaggttcacggggcccaattttaatggcacggtgttcctgggcaacaccaacttggtgctgcacggggtgagcttctatggctttaacaacacctgtgtggaggcctggaccgatgtgaaggtccgtggctgtgccttctacggatgttggaaggcggtagtgtgtcgccccaagagcaggagttccattaaaaaatgcttgtttgagaggtgcaccctgggggtgctggcggagggcaactgtcgggtgcgccacaatgtggcctcagaatgcggttgcttcatgctagtcaagagcgtggcggtcatcaagcataacatggtgtgcggcaacagcgaggacaaggcctcgcagatgctgacctgctcggatggcaactgccacttactgaagaccgtacatataaccagccacagccgcaaggcctggcccgtgttcgagcacaacgtgttgacccgctgctctttgcatctgggcaacaggaggggtgtgttcctgccctatcaatgcaacttgagccacaccaagatcttgctagagcccgaaagcatgtccaaggtgaacctgaacggggtgtttgacatgaccctgaagatatggaaggtgctgaggtacgacgagaccaggtctcgatgcaggccctgcgagtgcgggggcaagcatatgaggaaccagcctgtgatgctggatgtgaccgaggagctgaggcctgaccacttggttctggcctgcaccagggccgagtttggttctagcgatgaagacacagactgaggtgggtgagtgggcgtggtctgggggtgggaagcaatatataagttgggggtcttagggtctctgtgtctgttttgcagagggaccgccggcgccatgagcgggagcagtagcagcaacgccttggatggcagcatcgtgagcccttatttgacgacgcgcatgccccactgggccggggtgcgtcagaatgtgatgggctccagcatcgacggacgacccgtgctgcccgcaaattccgccacgctgacctacgcgaccgtcgcggggaccccgttggacgccaccgccgccgccgccgccaccgccgccgcctcggccgtgcgcagcctggccacggactttgcattcttgggacccttggccaccggggcggccgcccgtgccgccgttcgcgatgacaagctgaccgccctgctggcgcagttggatgcgcttacccgggaactgggtgacctttcgcagcaggtcgtggccctgcgccagcaggtctccgccctgcaggctagcgggaatgcttctcctgcaaatgccgtttaagataaataaaaccagactctgtttggattaaagaaaagtagcaagtgcattgctctctttatttcataattttccgcgcgcgataggcccgagtccagcgttctcggtcgttgagggtgcggtgtatcttctccaggacgtggtagaggtggctctggacgttgagatacatgggcatgagcccgtcccgggggtggaggtagcaccactgcagagcttcatgctccggggtggtgttgtagatgatccagtcgtagcaggagcgctgggcatggtgcctaaaaatgtccttaagcagcaggccgatggccagggggaggcccttggtgtaagtgtttacaaaacggttgagttgggaagggtgcatgcggggtgagatgatgtgcatcttagattgtatttttagattggcgatgtttcctcccagatcccttctgggattcatgttgtggaggaccaccagcacagtatatccggtgcacttgggaaatttgtcatgcagcttagagggaaatgcgtggaagaacttggagacgcccttgtggcctcccagattctccatgcattcgtccatgatgatggcaatgggcccgcgggaggcggcctgggcaaagatgtttctggggtcactgacatcgtagttgtgttccagggtgagatcgtcataggccatttttataaagcgcgggcggagggtgcccgactgggggatgatggttccctcgggccccggggcgtagttgccttcgcagatctgcatttcccaggccttaatctctgaggggggaatcatatccacttgcggggcgatgaagaaaacggtttccggagccggggagattaactgggatgagagcaggtttctcagcagctgtgactttccacagccggtgggtccataaataacacctataaccggctgcagctggtagttgagcgagctgcagctgccgtcgtcccggaggaggggggccacctcattgagcatgtcccggacgcgcttgttctcctcgaccaggtccgccagaaggcgctcgccgcccagggacagcagctcttgcaaggaagcaaagtttttcagcggtttgaggccgtccgccgtgggcatgtttttcagggtctggccgagcagctccaggcggtcccagagctcggtgacgtgctctacggcatctctatccagcatatctcctcgtttcgcgggttggggcggctttcgctgtagggcaccaggcgatggtcgtccagcgcggccagagtcatgtccttccatgggcgcagggtcctcgtcagggtggtctgggtcacggtgaaggggtgcgccccgggctgggcgctggccagggtgcgcttgagactggtcctgctggtgctgaagcgctgccggtcttcgccctgcgcgtcggccaggtagcatttgaccatggtgtcgtagtccagcccctccgcggcgtgtcccttggcgcgcagcttgcccttggaggtggcgccgcacgcggggcactgcaggctcttgagcgcgtagagcttgggggcgaggaagaccgattcgggggagtaggcgtccgcgccgcaggccccgcacacggtctcgcactccaccagccaggtgagctcggggcgctcggggtcaaaaaccaggtttcccccatgctttttgatgcgtttcttacctcgggtctccatgaggcggtgtccccgttcggtgacgaagaggctgtccgtgtctccgtagaccgacttgaggggtctgtcctccaggggggtccctcggtcctcttcgtagagaaactcggaccactctgagacaaaggcccgcgtccaggccaggacgaaggaggccaggtgggaggggtaccggtcgttgtccactagggggtccaccttctccaaggtgtgaagacacatgtcgccctcctcggcgtccaggaaggtgattggcttgtaggtgtaggccacgtgacccggggttccggacgggggggtataaaagggggtgggggcgcgctcgtcctcactctcttccgcatcgctgtctgcgagggccagctgctggggtgagtattccctctcgaaggcgggcatgacctcagcgctgaggctgtcagtttctaaaaacgaggaggatttgatgttcacctgtcccgagctgatgcctttgagggtgcccgcgtccatctggtcagaaaacacgatctttttattgtccagcttggtggcgaacgacccgtagagggcgttggagagcagcttggcgatggagcgcagggtctgattcttgtcccggtcggcgcgctccttggccgcgatgttgagctgcacgtactcgcgcgcgacgcagcgccactcggggaagacggtggtgcgctcgtcgggcaccaggcgcacgcgccagccgcggttgtgcagggtgacgaggtccacgctggtggcgacctcgccgcgcaggcgctcgttggtccagcagaggcgcccgcccttgcgcgagcagaaggggggcagggggtcgagttgggtttcgtccggggggtccgcgtccaccgtgaagaccccggggcgcaggcgcgcgtcgaagtagtcgatcttgcatccttgcaagtccagcgcccgctgccagtcgcgggcggcgagcgcgcgctcgtaggggttgagcggcgggccccagggcatggggtgggtgagcgcggaggcgtacatgccgcagatgtcatagacgtagaggggctcccggaggatgcccaggtaggtggggtagcagcggccgccgcggatgctggcgcgcacgtagtcgtagagctcgtgcgagggggcgaggaggtcggggcccaggttggtgcgggcggggcgctccgcgcggaagacgatctgcctgaagatggcatgcgagttggaagagatggtggggcgctggaagacgttgaagctggcgtcctgcaggccgacggcgtcgcgcacgaaggaggcgtaggactcgcgcagcttgtgcaccagctcggcggtgacctgcacgtcgagcgcgcagtagtcgagggtctcgcggatgatgtcatacttagcctgccccttctttttccacagctcgcggttgaggacgaactcttcgcggtctttccagtactcttggatcgggaaaccgtccggctccgaacggtaagagcccagcatgtagaactggttgacggcctggtaggcgcagcagcccttctccacgggcagggcgtaggcctgcgcggccttgcggagcgaggtgtgggtcagggcgaaggtgtccctgaccatgaccttgaggtactggtgtttgaagtcggagtcgtcgcagccgccccgctcccagagcgagaagtcggtgcgctttttggagcgggggttgggcagcgcgaaggtgacatcgttgtagaggatcttgcccgcgcgaggcatgaagttgcgggtgatgcggaagggccccggcacttccgagcggttgttgatgacctgggcggcgagcacgatctcgtcgaagccgttgatgttgtggcccacgatgtagagttccaggaagcggggccggcccttgacgctgggcagcttctttagctcttcgtaggtgagctcctcgggcgaggcgaggccgtgctcggccagggcccagtccgccaggtgcgggttgtccgcgaggaaggaccgccagaggtcgcgggccaggagggtctgcaggcggtccctgaaggtcctgaactggcggcctacggccatcttttcgggggtgacgcagtagaaggtgagggggtcttgctgccaggggtcccagtcgagctccagggcgaggtcgcgcgcggcggcgaccaggcgctcgtcgcccccgaatttcatgaccagcatgaagggcacgagctgctttccgaaggcgcccatccaagtgtaggtctctacatcgtaggtgacaaagagacgttccgtgcgaggatgcgagccgatcgggaagaactggatctcccgccaccagttggaggagtggctgttgatgtggtgaaagtagaagtcccgtcggcgggccgagcactcgtgctggcttttgtaaaagcgagcgcagtactggcagcgctgcacgggctgtacctcttgcacgagatgcacctgccgaccgcggacgaggaagctgagtgggaatctgagccccccgcatggctcgcggcctggctggtgctcttctactttggatgcgtggccgtcaccgtctggctcctcgaggggtgttacggtggagcggatcaccacgccgcgcgagccgcaggtccagatatcggcgcgcggcggtcggagtttgatgacgacatcgcgcagctgggagctgtccatggtctggagctcccgcggcggcggcaggtcagccgggagttcttgcaggtttacctcgcagagacgggccagggcgcggggcaggtccaggtggtacttgaattcgagaggcgtgttggtggcggcgtcgatggcttgcagtatgccgcagccccggggcgcgacgacggtgccccgcggggcggtgaagctcccgccgccgctcctgctgtcgccgccggtggcggggcttagaagcggtgccgcggtcgggcccccggaggtagggggggctccggtcccgcgggcaggggcggcagcggcacgtcggcgccgcgcgcgggcaggagctggtgctgcgcccggaggttgctggcgaaggcgacgacgcggcggttgatctcctggatctggcgcctctgcgtgaagacgacgggtccggtgagcttgaacctgaaagagagttcgacagaatcaatctcggtgtcattgaccgcgacctggcgcaggatctcctgcacgtcgcccgagttgtcttggtaggcgatctcggccatgaactgttcaatctcttcctcctggaggtctccgcgtccggcgcgctccacggtggccgccaggtcgttggagatgcgcgccatgagctgcgagaaggcgttgagtccgccctcgttccacactcggctgtagaccacgccgccctggtcgtcgcgggcgcgcatgaccacctgcgcgaggttgagttccacgtggcgcgcaaagacggcgtagttgcgcaggcgctggaagaggtagttgagggtggtggcggtgtgctcggccacaaagaagtacatgacccagcggcgcaacgtggattcgttgatgtcccccaaggcctccagtcgctccatggcctcgtagaagtccacggcgaagttgaaaaactgggagttgcgcgccgacacggtcaactcctcctccagaagacggatgagctcggcgacggtgtcgcgcacctcgcgctcgaaggctatgggaatctcttcctccgccagcatcaccacctcttcctcttcttcctcctctggcacttccatgatggcttcctcctcttcggggggtggcggcgggggagggggcgctcggcgccggcggcggcgcaccgggaggcggtccacgaagcgctcgatcatctccccgcggcggcgacgcatggtctcggtgacggcgcggccgttctctcggggacgcagctggaagacgccgccggtcatctggtgctggggcgggtggccgtggggcagcgagaccgcgctgacgatgcatcttaacaattgctgcgtaggtacgccgccgagggacctgagggagtccagatccaccggatccgaaaacctttcgaggaaggcatctaaccagtcgcagtcgcaaggtaggctgagcaccgtggcgggcggcggggggtggggggagtgtctggcggaggtgctgctgatgatgtaattgaagtaggcggtcttgacacggcggatggtcgacaggagcaccatatctttgggcccggcctgctggatgcggaggcggtcggccatgccccaggcttcgttctggcatctgcgcaggtctttgtagtagtcttgcatgagcctttccaccggcacctcttctccttcttcttctgacatctctgctgcatctgcggccctggggcgacggcgcgcgcccctgccccccatgcgcgtcaccccgaaccccctgagcggctggagcagggccaggtcggcgacgacgcgctcggccaggatggcctgctggacctgcgtgagggtggtttggaagtcatccaagtccacgaagcggtggtaggcgcccgtgttgatggtgtaggtgcagttggccatgacggaccagttgacggtctggtggcccggttgcgtcatctcggtgtacctgaggcgcgagtaggcgcgcgagtcgaagatgtagtcgttgcaagtccgcaccaggtactggtagcccaccaggaagtgcggcggcggctggcggtagaggggccagcggagggtggcgggggctccgggggccaggtcttccagcatgaggcggtggtattcgtagatgtacctggacatccaggtgatgcccgcggcggtggtggaggcgcgcgggaagtcgcgcacccggttccagatgttgcgcagcggcagaaagtgctccatggtaggcgtgctctggccggtcaggcgcgcgcagtcgttgatactctagaccagggaaaacgaaagccggtcagcgggcactcttccgtggtctggtggataaattcgcaagggtatcatggcggagggcctcggttcgagccccgggcccgggccggacggtccgccatgatccacgcggttaccgcccgcgtgtcgaacccaggtggcgacgtcagacaacggtggagtgttccttttgggttttttttccaaatttttctggccgggcgccgacgccgccgcgtaagagactagagtgcaaaagcgaaagcagtaagtggctcgctccctgtagcccggaggatccttgctaagggttgcgttgcggcgaaccccggttcgagtctggctctcgcgggccgctcgggtcggccggaaccgcggctaaggcgggattggcctccccctcattaaagaccccgcttgcggattcctccggacacaggggacgagcccctttttacttttgcttttctcagatgcatccggtgctgcggcagatgcgccccccgccccagcagcagcagcaacatcagcaagagcggcaccagcagcagcgggagtcatgcagggccccctcgcccacgctcggcggtccggcgacctcggcgtccgcggccgtgtctggagccggcggcggggggctggcggacgacccggaggagcccccgcggcgcagggccagacagtacctggacctggaggagggcgagggcctggcgcgactgggggcgccgtcccccgagcgccacccgcgggtgcagctgaagcgcgactcgcgcgaggcgtacgtgcctcggcagaacctgttcagagaccgcgcgggcgaggagcccgaggagatgcgggaccgcaggttcgccgcggggcgggagctgcggcaggggctgaaccgggagcggctgctgcgcgaggaggactttgagcccgacgcgcggacggggatcagccccgcgcgcgcgcacgtggcggccgccgacctggtgacggcatacgagcagacggtgaaccaggagatcaacttccaaaaaagcttcaacaaccacgtgcgcacgctggtggcgcgcgaggaggtgaccatcggcctgatgcacctgtgggactttgtgagcgcgctggagcagaaccccaacagcaagcctctgacggcgcagctgttcctgatagtgcagcacagcagggacaacgaggcgttcagggacgcgctgctgaacatcaccgagcccgagggtcggtggctcctggacctgattaacatcttgcagagcatagtggtgcaggagcgcagcctgagcctggccgacaaggtggcggccatcaattactcgatgctcagtctgggcaagttttacgcgcgcaaaatctaccagacgccgtacgtgcccatagacaaggaggtgaagatcgacggcttctacatgcgcatggcgctgaaggtgctgaccctgagcgacgacctgggcgtgtaccgcaacgagcgcatccacaaggccgtgagcgtgagccggcggcgcgagctgagcgaccgcgagctgatgcacagcctgcagcgggcgctggcgggggccggcagcggcgacagggaggccgagtcctacttcgaggcgggggcggacctgcgctgggtgcccagccggagggccctggaggccgcgggggcccgccgcgaggactatgcagacgaggaggaggaggatgacgaggagtacgagctagaggagggcgagtacctggactaaaccgcaggtggtgtttttggtagatgcaagacccgaacgtggtggacccggcgctgcgggcggctctgcagagccagccgtccggccttaactctacagacgactggcgacaggtcatggaccgcatcatgtcgctgacggcgcgcaatccggacgcgttccggcagcagccgcaggccaacaggctctccgccatcttggaggcggtggtgcctgcgcgcgcgaaccccacgcacgagaaggtgctggccatagtgaacgcgctggccgagaacagggccatccgcccggacgaggccgggctggtgtacgacgcgctgctgcagcgcgtggcccgctacaacagcggcaacgtgcagaccaacctggaccggctggtgggggacgtgcgcgaggcggtggcgcagcgggagcgcgcggagcggcagggcaacctgggctccatggtggcgctgaacgccttcctgagcacgcagccggccaacgtgccgcgggggcaggaggactacaccaactttgtaagcgcgctgcggctgatggtgaccgagaccccccagagcgaggtgtaccagtcggggccggactacttcttccagaccagcagacagggcctgcagacggtgaacctgagccaggctttcaagaacctgcgggggctgtggggggtgaaggcgcccaccggggaccgggcgacggtgtccagcctgctgacgcccaactcgcgcctgctgctgctgctgatcgcgccgttcacggacagcggcagcgtgtcccgggagacctacctcgggcacctgctgacgctgtaccgcgaggccatcgggcagacccaggtggacgagcacaccttccaggagatcaccagcgtgagccgcgcgctggggcaggaggacacgggcagcctggaggcgaccctgaactacctgctgaccaaccggcggcagaagatcccctcgctgcatagtttgaccaccgaggaggagcgcatcctgcgctacgtgcagcagagcgtgagcctgaacctgatgcgcgacggggtgacgcccagcgtggcgctggacatgaccgcgcgcaacatggaaccgggcatgtacgccgcgcaccggccttacatcaaccgcctgatggactacttgcatcgcgcggcggccgtgaaccccgagtacttcaccaacgccatcctgaacccgcactggctcccgccgcccgggttctacagcgggggcttcgaggtccccgaggccaacgacggcttcctgtgggacgacatggacgacagcgtgttctccccgcggccgcaggcgctggcggaggcgtcgctgctccgcctccccaagaaggaagagagccgccggcccagcagcgcggcggcctctctgtccgagctgggggcggcggccgcgcggcccgggtccctggggggcagcccctttcccagcctggtggggtctctgcagagcgggcgcaccacccggccccggctgctgggcgaggacgagtacctgaacaactccctgatgcagccggtgcgggagaaaaacctgccccccgccttccccaacaacgggatagagagcctggtagacaagatgagcagatggaagacctatgcgcaggagcacagggactcgcccgtgctccgtccgcccacgcggcgccagcgccacgaccggcagcgggggctggtgtgggatgacgaggactccgcggacgatagcagcgtgctggacctgggggggagcggcggcaacccgttcgcgcacctgcgcccccgcctggggaggatgtttcaataaaaaaaaaaaaaaatcaagcatgatgcaaggttttttaagcggataaataaaaaactcaccaaggccatggcgaccgagcgttgttggtttcttgttgtgttcccttagtatgcggcgcgcggcgatgtaccacgagggacctcctccctcttatgagagcgtggtgggcgcggcggcggcctctccctttgcgtcgcagctggagccgccgtacgtgcctccgcggtacctgcggcctacggggggaagaaacagcatccgttactcggagctggcgcccctgtacgacaccacccgggtgtacctggtggacaacaagtcggcggacgtggcctccctgaactaccagaacgaccacagcaattttttgaccacggtcatccagaacaatgactacaccccgagcgaggccagcacccagaccatcaatctggatgaccggtcgcactggggcggcgacctgaaaaccatcctgcacaccaacatgcccaacgtgaacgagttcatgttcaccaataagttcaaggcgcgggtgatggtgtcgcgctcgcacaccaaggacgaccgggtggagctgaagtacgagtgggtagagttcgagctgcccgagggcaactactcggagaccatgaccatagacctgatgaacaacgcgatcgtggagcactatctgaaagtgggcaggcagaacggggtcctggagagcgacatcggggtcaagttcgacaccaggaacttccgcctggggctggacccggtcaccgggctggttatgcccggggtctacaccaacgaggccttccaccccgacatcatcctgctgcccggctgcggggtggacttcacctacagccgcctgagcaacctgctgggcatccgcaagcggcagcccttccaggagggcttcaggatcacctacgaggacctggaggggggcaacatccccgcgctcctggatgtggaggcctaccaggatagcttgaaggaagaagaggcgggagagggcagcggcggtggcgccggtcaggaggagggcggggcctcctctgaggcctctgcggacccagccgctgccgccgaggcggaggcggccgaccccgcgatggtggtagaggaagagaaggatatgaacgacgaggcggtgcgcggcgacacctttgccactcggggggaggagaagaaagcggaggccgaggccgcggcagaggaggcggcagcagcggcggcggcagtagaggcggcggccgaggcggagaagccccccaaggagcccgtgattaagcccctgaccgaagatagcaagaagcgcagttacaacgtgctcaaggacagcaccaacaccgagtaccgcagctggtacctggcctacaactacggcgacccggcgacgggggtgcgctcctggaccctgctgtgtacgccggacgtgacctgcggctcggagcaggtgtactggtcgctgcccgacatgatgcaagaccccgtgaccttccgctccacgcggcaggtcagcaacttcccggtggtgggcgccgagctgctgcccgtgcactccaagagcttctacaacgaccaggccgtctactcccagctcatccgccagttcacctctctgacccacgtgttcaatcgctttcctgagaaccagattctggcgcgcccgcccgcccccaccatcaccaccgtcagtgaaaacgttcctgctctcacagatcacgggacgctaccgctgcgcaacagcatcggaggagtccagcgagtgaccgtaactgacgccagacgccgcacctgcccctacgtttacaaggccctgggcatagtctcgccgcgcgtcctttccagccgcactttttaagcatgtccatcctcatctcgcccagcaataacaccggctggggcctgctgcgcgcgcccagcaagatgttcggaggggcgaggaagcgctccgaccagcaccccgtgcgcgtgcgcgggcactaccgcgctccctggggcgcgcacaaacgcgggcgcaccggcaccgcggggcgcaccaccgtggacgaagccatcgactcggtggtggagcaggcgcgcaactacacgcccgcggtctccaccgtggacgcggctatcgagagcgtggtgcgaggcgcgcggcggtacgccaaggcgaagagccgccggaggcgcgtggcccgccgccaccgccgccgacccgggagcgccgccaagcgcgccgccgccgccttgctccgtcgggccagacgcacgggccgccgtgccgccatgagggccgcgcgccgcctggccgccggcatcaccaccgtggccccccgcgccagaagacgcgcggccgccgccgccgccgcggccatcagcgacctggccaccaggcgccggggcaacgtgtactgggtgcgcgactcggtgagcggcacgcgcgtgcccgtgcgcttccgccccccgcggacttgagaggagaggacaggaaaaagcaacaacatcaacaacaccaccactgagtctcctgctgttgtgtgtatcccagcggcgcgcgcgcacacggcgacatgtccaagcgcaaaatcaaagaagagatgctccaggtcgtcgcgccggagatctatgggcccccgaagaaggaagagcaggatttcaagccccgcaagataaagcgggtcaaaaagaaaaagaaagatgacgatgatggcgaggtggagtttctgcgcgccacggcgcccaggcgcccgctgcagtggaagggtcggcgcgtaaagcgcgttctgcgccccggcaccgcggtggtcttcacgcccggcgagcgctccacccgcactttcaagcgcgtctatgacgaggtgtacggcgacgaagacctgctggagcaggccaacgatcgctccggagagtttgcttacgggaagcggcaccgggcgatggagaaggacgaggtgctggcgctgccgctggaccggggcaaccccacccccagcctgaagcccgtgaccctgcagcaggtgctaccggccagcgcgccctccgagatgaagcggggcctgaagcgcgagggcggcgacctggcgcccaccgtgcagctaatggtgcccaagcggcagaggctggaggacgtgctggagaaaatgaaagtagaccccggcctgcagccggacatcagggtccgccccatcaagcaggtggcgccgggcctcggcgtgcagaccgtggacgtggtcatccccaccggcgcctcctcttccagcgccgccgccgccgccactagcaccgcggacatggagacgcagactagccccgccgccacctcctcggcggaggtacagacggacccctggttgccgccgccggcgaccgccccctcgcgcgcacgccgcgggcgcaggaagtacggcgccgccagcgcgctcatgcccgagtacgccttgcatccttccatcgcgcccacccccggctaccgaggctacagttaccgcccgcgaagagccaagggctccacccgccgcagccgccgcgccgccacctctacccgccgccgcagtcgccgccgccgccggcagcccgcgctggctccgatctccgtgaaaagagtggcgcgcaacgggaacaccttggtgctgcccagggcgcgctaccaccccagcatcgtttaaaaagcctgttgtggttcttgcagatatggccctcacttgccgcctccgtttcccggtgccgggataccgaggaagatcgcgccgcaggaggggtatggccggacgcggcctgagcggaggcagtcgccgtgcgcaccggcggcgacgcgccaccagccgacgcatgcgcggcggagtgctgcctctgctgatccccctgatcgccgcggcgatcggcgccgtgcccgggatcgcctccgtggccttgcaggcgtcccagaggcgttgacacagacttcttgcaagcttgcaaaaatatggaaaaatccccccaataaaaaagtctagactctcacgctcgcttggtcctgtgactattttgtagaaaaaagatggaagacatcaactttgcgtcgctggccccgcgtcacggctcgcgcccgttcctgggacactggaacgatatcggcaccagcaacatgagcggtggcgccttcagttggggctctctgtggagcggcattaaaaatatcggttctgccgttaagaattacggcaccaaggcctggaacagcagcacgggccagatgttgagagacaagttgaaagagcagaacttccagcagaaggtggtggagggtctggcctccggcatcaacggggtggtggacctggccaatcaggccgtgcaaaataagatcaacagcagactggacccccggccgccggtggaggagctgccgccggcgctggagacggtgtcccccgatgggcggggcgaaaagcgcccgcggcccgacagggaagagaccactctggtcacgcacaccgatgagccgcccccctacgaggaagccctgaagcaaggcttgcccaccactcggcccatcgcgcccatggccaccggggtggtgggccgccacacccccgccacgctggacctgcctcctcctcctgtttcttcttcggccgccgatgcgcagcagcagaaggcggcgctgcccggtccgcccgcggccgccccccgtcccaccgccagtcgagcgcccctgcgtcgcgcggccagcggcccccgcggggtcgcgaggcacagcagcggcaactggcagaacacgctgaacagcatcgtgggtctgggggtgcagtccgtgaagcgccgccgatgctactgaatagcttagctaacggtgttgtatgtgtgtatgcgtcctatgtcaccgccagaggagctgctgagtcgccgccgttcgcgcgcccaccgccactaccaccgccggtaccactccagcgcccctcaagatggcgaccccatcgatgatgccgcagtggtcgtacatgcacatctcgggccaggacgcctcggagtacctgagccccgggctggtgcagttcgcccgcgccaccgacagctacttcagcctgagtaacaagtttaggaaccccacggtggcgcccacgcacgatgtgaccaccgaccggtcccagcgcctgacgctgcggttcatccccgtggaccgcgaggacaccgcgtactcttacaaggcgcggttcaccctggccgtgggcgacaaccgcgtgctggacatggcctccacctactttgacatccgcggcgtgctggacaggggccccaccttcaagccctactccggcaccgcctacaactccctggcccccaagggcgcccccaactcctgcgagtgggagcaagtggagccagctgaagaggcagcagaaaatgaagatgaagaagaagaagaggatgttgttgatcctcaggaacaggagcccactactaaaacacatgtatatgctcaagctcccctttctggcgagaaaattaccaaagatggtctgcaaataggaactgaggctacggcagcaggaggcactaaagacttatttgcagaccctacattccagccagaaccccaagttggcgaatctcagtggaatgaggcggatgctacagcagctggaggtagagtgctcaaaaagaccactcccatgaaaccttgctatggctcatatgcccgccccacaaatgccaatgggggccaaggtgtgctaaaggcaaatgcccagggagtgctcgagtctcaggttgagatgcagttcttttccacttctacaaatgccacaaacgagcaaaacaacatccagcccaaattggtgctgtacagcgaggatgtgcatatggagaccccagacacacacatctcctacaagcctacaaaaagcgatgataattcaaaagtcatgctgggtcagcagtccatgcccaacaggccaaattacatcgccttcagagacaactttatcgggctcatgtattataacagcactggcaacatgggggtgctggcaggtcaggcctcacagttgaatgcagtggtggacctgcaagacagaaacacagaactgtcctaccagctcttgcttgattccatgggagacagaaccagatacttttccatgtggaatcaggccgtggacagttatgacccagatgtcagaattattgaaaatcatggaaccgaagatgagctgcccaactattgtttccctctgggaggcatagggataactgacacttaccaggccattaagactaatggcaatggggcaggagatcaagccaccacgtggcagaaagactcacaatttgcagaccgcaacgaaataggggtgggaaacaacttcgccatggagatcaacctcagtgccaacctgtggaggaacttcctctactccaacgtggccctgtacctgccagacaagcttaagtacaacccctccaacgtggaaatctctgacaaccccaacacctacgactacatgaacaagcgagtggtggccccggggctggtggactgctacatcaacctgggcgcgcgctggtccctggactacatggacaacgtcaaccccttcaaccaccaccgcaatgcgggcctgcgctaccgctccatgcttctgggcaacgggcgctacgtgcccttccacatccaggtgccccagaagttctttgccatcaagaacctcctcctcctgccgggctcctacacctacgagtggaacttcaggaaggatgtcaacatggtcctgcagagctctctgggcaacgacctcagggtcgacggggccagcatcaagttcgagagcatctgcctctacgccaccttcttccccatggcccacaacacggcctccacgctcgaggccatgctcaggaacgacaccaacgaccagtccttcaacgactacctctccgccgccaacatgctctaccccatccccgccaacgccaccaacgtccccatctccatcccctcgcgcaactgggcggccttccgcggctgggccttcacccgccttaagaccaaggagaccccctccctgggctcgggtttcgacccctactacacctactcgggctccataccctacctggacggaaccttctacctcaaccacactttcaagaaggtctcggtcaccttcgactcctcggtcagctggccgggcaacgaccgcctgctcacccccaacgagttcgagatcaagcgctcggtcgacggggagggctacaacgtagcccagtgcaacatgaccaaggactggttcctcatccagatgctggccaactacaacatcggctatcagggcttctacatcccagagagctacaaggacaggatgtactccttctttaggaacttccagcccatgagccggcaggtggtggacgaaaccaagtacaaggactaccagcaggtgggcatcatccaccagcacaacaactcgggcttcgtgggctacctcgcccccaccatgcgcgagggacaggcctaccccgccaacttcccctacccgctcattggcaagaccgcggtcgacagcatcacccagaaaaagttcctctgcgaccgcaccctctggcgcatccccttctccagcaacttcatgtccatgggtgcgctcacggacctgggccagaacctgctctatgccaactccgcccacgcgctcgacatgaccttcgaggtcgaccccatggacgagcccacccttctctatgttctgttcgaagtctttgacgtggttcgggtccaccagccgcaccgcggcgtcatcgagaccgtgtacctgcgcacgcccttctcggccggcaacgccaccacctaaagaagcaagccgccaccgccaccacctgcatgtcgtcgggttccaccgagcaggagctcaaggccatcgtcagagacctgggatgcgggccctattttttgggcaccttcgacaaacgcttcccgggcttcgtcgccccgcacaagctggcctgcgccatcgtcaacacggccggccgcgagaccgggggcgtgcactggctggccttcgcctggaacccgcgctccaaaacatgctacctctttgaccccttcggattctcggaccagcggctcaagcagatctaccagttcgagtacgagggcctgctgcgccgcagcgccatcgcctcctcgcccgaccgctgcgtcaccctcgagaagtccacccagaccgtgcaggggcccgactcggccgcctgcggtctcttctgctgcatgttcctgcatgcctttgtgcactggccccagagtcccatggaccgcaaccccaccatgaacttgctgacggggatccccaactccatgctccagagcccccaggccgcgcccaccctgcgccgcaaccaggagcggctctacagcttcctggagcgccactcgccctacttccgccgccacagcgcgcagatcaggggggccacctctttctgccgcatgcaagagatgcaagggaaaatgcaatgatgtacacagacactttctttttctcaataaatggcaactttatttatacatgctctctctcgggtattcatttccccaccacccaccacccgccgccgtaaccatctgctgctggctttttaaaaatcgaaagggttctgccgggaatcgccgtgcgccacgggcagggacacgttgcggaactggtagcgggtgccccacttgaactcgggcaccaccatgcggggcaagtcggggaagttgtcggcccacaggccgcgggtcagcaccagcgcgttcatcaggtcgggcgccgagatcttgaagtcgcagttggggccgccgccctgcgcgcgcgagttgcggtacaccgggttgcaacactggaacaccagcagcgccggataattcacgctggccagcacgctccggtcggagatcagctcggcgtccaggtcctccgcgttgctcagcgcgaacggggtcagcttgggcacctgccgccccaggaagggagcgtgccccggcttcgagttgcagtcgcagcgcagcgggatcagcaggtgcccgcggccggactcggcgttggggtacagcgcgcgcatgaaggcctccatctggcggaaggccatctgggccttggcgccctccgagaagaacatgccgcaggacttgcccgagaactggttcgcggggcagctagcgtcgtgcaggcagcagcgcgcgtcggtgttggcaatctgcaccacgttgcgcccccaccggttcttcacgatcttggccttggaagcctgctccttcagcgcgcgctgcccgttctcgctggtcacatccatctcgatcacgtgctccttgttcaccatgctgctgccgtgcagacacttcagctcgccctccacctcggtgcagcggtgctgccacagcgcgcagcccgtgggctcgaaatgcttgtaggtcacctccgcgtaggactgcaggtaggcctgcaggaagcgccccatcatggtcacgaaggtcttgttgctgctgaaggtcagctgcagcccgcggtgctcctcgttcagccaggccttgcacacggccgccagcgcctccacctggtcgggcagcatcttgaagttcagcttcagctcattctccacatggtacttgtccatcagcgcgcgcgcagcctccatgcccttctcccaggccgacaccagcggcaggctcaaggggttcaccaccgtcgcagtcgccgccgcgctttcgctttccgctccgctgttctcttcttcctcctcctcttcttcctcgccgcccgcgcgcagcccccgcaccacggggtcgtcttcctgcaggcgccgcaccgagcgcttgccgctcctgccctgcttgatgcgcacgggcgggttgctgaagcctaccatcaccagcgcggcctcttcttgctcgtcctcgctgtccactatgacctcgggggagggcgacctcagaaccgtggcgcgctgcctcttctttttcctgggggcgtttgcaagctccgcggccgcggccgccgccgaggtcgaaggccgagggctgggcgtgcgcggcaccagcgcgtcctgcgagccgtcctcgtcctcggactcgaggcggcagcgagcccgcttctttgggggcgcgcggggcggcggcggcgggggcggcggcgacggagacggggacgagacatcgtccagggtgggaggacggcgggccgcgccgcgtccgcgctcgggggtggtttcgcgctggtcctcttcccgactggccatctcccactgctccttctcctataggcagaaagagatcatggagtctctcatgcaagtcgagaaggaggaggacagcctaaccaccgccccctctgagccctccgccgccaccgccgcggacgacgcgcctaccaccgccgccaccaccaccaccattaccaccctacccggcgacgcagccccgatcgagaaggaagtgttgatcgagcaggacccgggttttgtgagcgaagaggaggatgaggaggatgaaaaggagaaggataccgccgcctcagtgccaaaagaggataaaaagcaagaccaggacgacgcagagacagatgaggcagcaatcgggcggggggacgagaggcatgatgatgatgatgatgacggctacctagacgtgggagacgacgtgctgcttaagcacctgcaccgccagtgcgtcatcgtctgcgacgcgctgcaggagcgctgcgaagtgcccctggacgtggcggaggtcagccgcgcctacgagcggcacctcttcgcgccacacgtgccccccaagcgccgggagaacggcacctgcgagcccaacccgcgcctcaacttctacccggtcttcgcggtacccgaggtgctggccacctaccacatcttcttccaaaactgcaagatccccctctcctgccgcgccaaccgcacccgcgccgacaaggcgctggccctgcggcagggcgcccacatacctgatatcgcctctctggaggaggtgcccaagatcttcgagggtctcggtcgcgacgagaaacgggcggcgaacgctctgcaaggagacagcgaaaacgagagtcactcgggggtgctggtggagctcgagggcgacaacgcgcgcctggccgtgctcaagcgcagcatcgaagtcacccacttcgcctacccggcgctcaacctgccccccaaggtcatgagtgtggtcatgagcgagctcatcatgcgccgcgcccagcccctggacgcggatgcaaacttgcaagagccctccgaggaaggcctgcccgcggtcagcgacgagcagctggcgcgctggctggagacccgcgaccccgcccagctggaggagcggcgcaagctcatgatggccgcggtgctcgtcaccgtggagctcgagtgtctgcagcgcttcttcggggaccccgagatgcagcgcaagctcgaggagaccctgcactacaccttccgccagggctacgtgcgccaggcctgcaagatctccaacgtggagctctgcaacctggtctcctacctgggcatcctgcacgagaaccgcctcgggcagaacgtcctgcactccaccctcaaaggggaggcgcgccgcgactacgtccgcgactgcgtctacctcttcctctgctacacgtggcagacagccatgggggtctggcagcagtgcctggaggagcgcaacctcaaggagctggagaagctcctcaggcgcgccctcagggacctctggaggggcttcaacgagcgctcggtggccgccgcgctggcggacatcatcttccccgagcgcctgctcaaaaccctgcagcagggcctgcccgacttcaccagccagagcatgctgcagaacttcaggaccttcatcctggagcgctcgggcatcctgccggccacctgctgcgcgctgcccagcgacttcgtgcccatcaggtacagggagtgcccgccgccgctctggggccactgctacctcttccagctggccaactacctcgcctaccactcggatctcatggaagacgtgagcggcgagggcctgctcgagtgccactgccgctgcaacctgtgcacgccccaccgctctctagtctgcaacccgcagctgctcagcgagagtcagattatcggtaccttcgagctgcagggtccctcgcccgacgaaaagtccgcggctccggggttgaaactcactccggggctgtggacttccgcctacctacgcaaatttgtacctgaagactaccacgcccacgagatcaggttttacgaagaccaatcccgcccgcccaaggcggagctcaccgcctgcgtcattacccagggccacatcctgggccaattgcaagccatcaacaaagcccgccaagagttcttgctgaaaaagggtcggggggtgtacctggacccccagtccggcgaggagctaaacccgctacccccgccgccgccccagcagcgggaccttgcttcccaggatggcacccagaaagaagcagccgccgccgccgccagcatacatgcttctggaggaagaggaggactgggacagtcaggcagaggaggtttcggacgaggacgaggaggaggagatgatggaagactgggaggaggacagcctagacgaggaagcttcagaggccgaagaggtggcagacgcaacaccatcaccctcggccgcagccccctcgccggcgcccccgaaatcctccgaccccagcagcagcgctataacctccgctcctccggcgccggcgcccacccgcagcagacccaaccgtagatgggacactacaggaaccggggtcggtaagtccaagtgccccccagcgccgcccccgcaacaggagcaacagcagcagcagcggcgacagggctaccgctcgtggcgcggacacaagaacgccatagtcgcctgcttgcaagactgcgggggcaacatctccttcgcccgccgcttcctgctcttccaccacggggtggcttttccccgcaatgtcctgcattactaccgtcatctctacagcccctactgcggcggcagcggcgacccagagggagcggcggcagcagcagcgccagccacagcggcgaccacctaggaagacctccgcgggcaagacggcgggagccgggagacccgcggcggcggcggtagcggcggcggcgggcgcactgcgcctctcgcccaacgaacccctctcgacccgggagctcagacacaggatcttccccactctgtatgctatcttccagcagagcagaggccaggaacaggagctgaaaataaaaaacagatctctgcgctccctcacccgcagctgtctgtatcacaaaagcgaagatcagcttcggcgcacgctggaggacgcggaggcactcttcagcaaatactgcgcgctgactcttaaggactagccgcgcgcccttctcgaatttaggcgggagaaagactacgtcatcgccgaccgccgcccagcccacccagccgacatgagcaaagagattcccacgccctacatgtggagctaccagccgcagatgggactcgcggcgggagcggcccaagactactccacccgcatgaactacatgagcgcggggccccacatgatctcacgggttaatgggatccgcgcccagcgaaaccaaatactgctggaacaggcggccataaccgccacaccccgtcatgacctcaatccccgaaattggcccgccgccctcgtgtaccaggaaaccccctctgccaccaccgtggtacttccgcgtgacacccaggccgaagtccagatgactaactcaggggcgcagctcgcgggcggctttcgtcacggggtgcggccgcaccggccgggtatattacacctggcgatcagaggccgaggtattcagctcaacgacgagtcggtgagctcttcgctcggtctccgtccggacggaaccttccagatcgccggatcaggtcgctcctcattcacgcctcgccaggcgtacctgactctgcagacctcctcctcggagcctcgctccggcggcatcggcaccctccagttcgtggaggagttcgtgccctcggtctacttcaaccccttctcgggacctcccggacgctaccccgaccagttcatcccgaactttgacgcggtgaaggactcggcggacggctacgactgaatgtcaagtgctgaggcagagagcgttcgcctgaaacacctccagcactgccgccgcttcgcctgctttgcccgcagctccggtgagttctgctactttcagctgcccgaggagcataccgaggggccggcgcacggcgtccgcctaaccacccagggcgaggttacctgtacccttatccgggagtttaccctccgtcccctgctagtggagcgggagcggggttcttgtgtcataactatcgcctgcaactgccctaaccctggattacatcaagatctttgttgtcacctgtgcgctgagtataataaacgctgagatcagactctactggggctcctgtcgccatcctgtgaacgccaccgtcttcacccaccccgagcagccccaggcgaacctcacctgcggcctgcgtcggagggccaagaagtacctcacctggtacttcaacggcaccccctttgtggtttacaacagcttcgaccaggacggagttgccttgagagacgacctttccggtctcagctactccattcacaagaacaccaccctccacctcttccctccctacctgccgggaacctacgagtgcgtcaccggccgctgcacccacctcctccgcctgatcgtaaaccagacctttccgggaacacacctcttccccagaacaggaggtgagctcaggaaaccccctggggcccagggcggagacttaccttcgacccttgtggggttaggattttttatcgccgggttgctggctctcctgatcaaagcttccttgagatttgttctctccctttacttttatgaacagctcaacttctaataacgctaccttttctcaggaatcgggtagtaacttctcttctgaaatcgggctgggtgtgctgcttactctgttgatttttttccttatcatacttagccttctgtgcctcaggctcgccgcctgctgcgcacatatctacatctacagccggttgcttaactgctggggtcgccatccaagatgaacggggctcaggtgctatgtctgctggccctggtggcctgcagtgccgccgtcaattttgaggaacccgcttgcaatgtgactttcaagcctgaaggcgcacattgcaccactctggttaaatgtgtgacctctcatgagaaactgctcatcgcctacaaaaacaaaacaggcgagttcgcggtctatagcgtgtggcaacccggagaccataataactactcagtcaccgtcttcgagggtgcggagtctaagaaattcgattacacctttcccttcgaggagatgtgtgaagcggtcatgtacctgtccaaacagtacaagctgtggccccccacccccgaggcgtgtgtggaaaacactgggtctttctgctgtctctctctgacaatcactgtgcttgctctaatctgcacgctgctgtacatgaaattcaggcagaggcgaatctttatcgatgagaaaaaaatgccttgatcgctaacaccggctttctgtctgcagaatgaaagcaatcacctccctactaatcagcaccaccctccttgcgattgcccatgggttgacacgaatcgaagtgccagtggggtccaatgtcaccatggtgggccccgccggcaattcctccctgatgtgggaaaaatatgtccgtaatcaatgggatcattactgctctaatcgaatctgtatcaagcccagagccatctgcgacgggcaaaatctaactttgattgatgtgcaaatgacggatgctgggtactattacgggcagcggggagaaatgattaattactggcgaccccacaaggactacatgctgcatgtagtcaaggcagtccccactactaccacccccaccactaccactcccaccactcccactactaccacccccaccactactactagcactgctactaccgctgcccgcaaagctattacccgcaaaagcaccatgcttagcaccaagccccattctcactcccacgccggcgggcccaccggtgcggcctcagaaaccaccgagctttgcttctgccaatgcactaacgccagcgcccacgaactgttcgacctggagaatgaggatgatgaccagctgagctccgcttgcccggtcccgctgcccgcagagccggtcgccctgaagcagctcggtgatccatttaatgactctcctgtttatccctctcccgaatacccgcccgactctaccttccacatcacgggcaccaacgaccccaacctctccttctacctgatgctgctgctttgtatctctgtggtatcttccgcgctcatgttactgggcatgttctgctgcctcatctgccgcagaaagagaaagtctcgctctcagggccaaccactgatgcccttcccctaccccccagattttgcagataacaagatatgagcacgctgctgacactaaccgctttactcgcctgcgctctaacccttgtcgcttgcgaatccagataccacaatgtcacagttgtgacaggagaaaatgttacattcaactccacggccgacacccagtggtcgtggagcggccacggtagctatgtatacatctgcaatagctccacctcccctagcatgtcctctcccaagtaccactgcaatgccagcctgttcaccctcatcaacgcctccacctcggacaatggactctatgtaggctatgtgacacccggtgggcggggaaagacccacgcctacaacctgcaagttcgccacccctccaccaccgccaccacctctgccgcccctacccgcagcagcagcagcatcagcagcagcagcagcagcagcagattcctgactttaatcctagccagctcaacaaccaccgccaccgctgagaccacccacagctccgcgcccgaaaccacccacacccaccacccagagacgaccgcggcctccagtgaccagatgtcggccaacatcaccgcctcgggtcttgaacttgcttcaacccccaccccaaaaccagtggatgcagccgacgtctccgccctcgtcaatgactgggcggggctgggaatgtggtggttcgccataggcatgatggcgctctgcctgcttctgctctggctcatctgctgcctcaaccgcaggcgggccagacccatctatagacccatcattgttctcaaccccgctgatgatgggatccatagattggatggtctgaaaaacctacttttctcttttacagtatgataaattgagacatgcctcgcattttcatgtacttgacacttctcccactttttctggggtgttctacgctggccgccgtctctcacctcgaggtagactgcctcacacccttcactgtctacctgatttacggattggtcaccctcactctcatctgcagcctaatcacagtagtcatcgccttcatccagtgcattgactacatctgtgtgcgcctcgcatacctgagacaccacccgcagtaccgagacaggaacattgcccaactcctaagactgctctaatcatgcataagactgtgatctgcctcctcatcctcctctccctgcccgctctcgtctcatgccagcccgccacaaaacctccacgaaaaagacatgcctcctgtcgcttgagccaactgtggaatattcccaaatgctacaatgaaaagagcgagctttccgaagcctggctatatgcggtcatgtgtgtccttgtcttctgcagcacaatctttgccctcatgatctacccccactttgatttgggatggaatgcggtcgatgccatgaattaccctacctttcccgcgcccgatatgattccactccgacaggttgtggtgcccgtcgccctcaatcaacgccccccatcccctacacccactgaggtcagctactttaatctaacaggcggagatgactgacactctagatctagaaatggacggcatcggcaccgagcagcgtctcctacagaggcgcaagcaggcggctgaacaagagcgcctcaatcaggagctccgagatctcattaacctgcaccagtgcaaaaaaggcatcttttgcctggtcaagcaggccgatgtcacctacgagaaaaccggtaacagccaccgcctcagctacaagctgcccacccaacgccagaagttggtgctcatggtgggtcagaatcccatcaccgtcacccagcactcggtggagaccgaggggtgtctgcactccccctgtcagggtccggaagacctctgcaccctggtaaagaccctgtgtggtcttagagatttaatcccctttaactaatcaaacactggaatcaataaaaagaatcacttactttaaatcagtcagcaggtctctgtccactttattcagcagcacctccttcccctcctcccaactctggtactccaaacgcctcctggcggcaaacttcctccacaccctgaagggaatgtcagattcttgctcctgtccctccgcacccactatcttcatgttgttgcagatgaagcgcgccaaaacgtctgacgagaccttcaaccccgtgtacccctatgacacggaaaacgggcctccctccgtccctttcctcacccctcccttcgtgtcccccgacggatttcaagaaagccccccaggggtcctgtctctgcgcctgtcagagcccctggtcacttcccacggcatgcttgccctgaaaatgggaaatggcctctccctggatgacgccggcaacctcacctctcaagatgtcaccaccgtcacccctcccctcaaaaaaaccaagaccaacctcagcctccagacctcagcccccctgaccgttagctctgggtccctcaccgtcgcggccgccgctccactggcggtggccggcacctctctcaccatgcaatctcaggcccccttgacagtgcaagatgcaaaactcggcctggccacccagggacccctgaccgtgtctgaaggcaaactcaccttgcagacatcggctccactgacggccgctgacagcagcactctcactgttagtgccacacctcccctcagcacaagcaatggtagtttgagcattgacatgcaggccccgatttataccaccaatggaaaactggcacttaacattggtgctcccctgcatgtggtagacaccctaaatgcactaactgtagtaactggccagggtcttaccataaatggaagagccctgcaaactagagtcacgggtgccctcagttatgacacagaaggcaacatccaactgcaagccggagggggtatgcgcattgacaataatggccaacttatccttaatgtagcttatccatttgatgctcaaaacaacctcagccttagacttggccaaggtcccctaattgttaactctgcccacaacttggatcttaaccttaacagaggcctttacttatttacatctggaaacacgaaaaaactggaagttaacataaaaacagccaaaggtctattttacgatggcaccgctatagcaatcaatgcaggtgacgggctacagtttgggtctggttcagatacaaatccattgcaaactaaacttggattggggctggaatatgactccaacaaagctataatcactaaacttggaactggcctaagctttgacaacacaggtgccatcacagtaggcaacaaaaatgatgacaagcttaccttgtggaccacaccagacccctccccaaactgcagaattaattcagaaaaagatgctaaactcacactagttttgactaaatgcggcagccaggtgttagccagcgtttctgttttatctgtaaaaggcagccttgcccccatcagcggcacagtaactagcgcccagattgttttaagatttgatgaaaacggagttttattgagcaattcttctcttgacccccaatactggaactatagaaaaggcgattctacagaaggcactgcatatactaatgctgtgggatttatgcccaacctcacagcataccctaaaacacagagccagactgctaaaagcaacattgtaagtcaagtttacttgaatggggacaaaacaaaacccatgaccctaaccatcaccctcaatggaactaatgaaacaggggatgctacagtaagcacatactccatgtcattttcatggaactggaatggaagtaattacattaatgacaccttccaaaccaactcctttaccttctcctacatcgcccaagaataaaaaagcatgacgctttgttctctgattcagtgtgtttcttttattttttttcaattacaacagaatcattcaagtcattctccatttagcttaatagacccagtagtgcaaagccccatactagcttatttcagacagtataaattaaaccataccttttgatttcaatattaaaaaaatcatcacaggatcctagtcgtcaggccgccccctccctgccaagacacagaatacacaatcctctccccccggctggctttaaacaacaccatctggttggtgacagacaggttcttcggggttatattccacacggtctcctggcgggccaggcgctcgtcggtgatgctgataaactctcccggcagctcgctcaagttcacgtcgctgtccagcggctgaacctcatgctgacgcggtaactgcgcgaccggctgctgaacaaacggaggccgcgcctacaagggggtagagtcataatcctccgtcaggatagggcggttatgcagcagcagcgagcgaatcatctgctgccgccgccgctccgtccggcaggaaaacaacatcccggtggtctcctccgctataatccgcaccgcccgcagcataagcctcctcgttctccgcgcgcagcaccgcaccctgatctcactcaggttggcgcagtaggtacagcacatcaccacgatgttattcatgatcccacagtgcaaggcgctgtatccaaagctcatgcccgggaccaccgcccccacgtgaccgtcgtaccagaagcgcaggtaaatcaagtgccgacccctcatgaacgtgctggacataaacatcacctccttgggcatgttgtaattcaccacctcccggtaccagatgaatctctgattgaacacggccccttccaccaccatcctgaaccaagaggctaggacctgcccaccggctatgcactgcagggaacccgggttagaacaatgacaatgcagactccagggctcgtaaccgtggatcatccggctgctgaagacatcgatgttggcgcaacacagacacacgtgcatacacttcctcatgattagcagctcctccctcgtcaggatcatatcccaagggataacccattcttgaatcaacgtaaagcccacagagcagggaaggcctcgcacataactcacgttgtgcatggttagcgtgttgcattccggaaacagcggatgatcctccagtatcgaggcgcgggtctcgttctcacagggaggtaaaggggccctgctgtacggactgtggcgggacgaccgagatcgtgttgagcgtaacgtcatggaaaagggaacgccggacgtggtcatacttcttgaagcagaaccaggctcgcgcgtgacagacctccttgcgtctacggtctcgccgcttagctcgctccgtgtgatagttgtagtacagccactctctcaaagcgtcgaggcgacacctggcgtcaggatgtatgtagactccgtcttgcaccgcggccctgataatatccaccaccgtagaataagccacaccaagccaagcaatacactcgctttgcgagcggcagacaggaggagcggggagagacggaaggaccatcataaaattttaaagaatattttccaatacttcgaaatcaagatctaccaaatggcaacgctcccctccactggcgcggtcaaactctacggccaaagaacagataacggcatttttaagatgttcccggacggcgtctaaaagacaaaccgctctcaagtcgacataaattataagccaaaagccatcgggatccatatccactatggacgcgccggcggcgtccaccaaacccaaataattttcttctctccagcgcagcaaaatcccagtaagcaactccctgatattaagatgaaccatgccaaaaatctgttcaagagcgccctccaccttcattctcaagcagcgcatcatgattgcaaaaattcaggttcctcagacacctgtatgagattcaaaacgggaatattaacaaaaattcctctgtcgcgcagatcccttcgcagggcaagctgaacataatcagacaggtctgaacgaaccagcgaggccaaatccccgccaggaaccagatccagagaccctatgctgattatgacgcgcatactcggggctatgctaaccagcgtagcgccgatgtaggcgtgctgcatgggcggcgaaataaaatgcaaggtgctggttaaaaaatcaggcaaagcctcgcgcaaaaaagctaagacatcataatcatgctcatgcaggtagttgcaggtaagctcaggaaccaaaacggaataacacacgattttcctctcaaacatgacttccaggtgactgcataagaaaaaaattataaataataaatattaattaaataaattaaacattggaagcctgtctcacaacaggaaaaaccactctgatcaacataagacgggccacgggcatgcccgcgtgaccataaaaaaatcggtctccgtgattacaaagcaccacagatagctccccggtcatgtcgggggtcatcatgtgagactgtgtatacacgtccgggctgttgacatcggtcaaagaaagaaatcgagctacatagcccggaggaatcaacacccgcacgcggaggtacagcaaaacggtccccataggaggaatcacaaaattagtaggagaaaaaaaaacataaacaccagaaaaaccctcttgccgaggcaaaacagcgccctcccgttccaaaacaacataaagcgcttccacaggagcagccatgacaaagacccgagtcttaccaggaaaattttaaaaaagattcctcaacgcagcaccagcaccaacacctgtcagtgtaaaatgccaagcgccgagcgagtatatataggaataaaaagtgacgtaaacggttaaagtccagaaaacgcccagaaaaaccgcacgcgaacctacgccccgaaacgaaagccaaaaaacagtgaacacgccctttcggcgtcaacttccgctttcccacggtacgtcacttccgcatatagtaaaactacgctacccaacatgcaagaagccacgccccaaaacacgtcacacctcccggcccgccccgcgccgccgctcctccccgccccgccccgctccgcccacctcattatcatattggcttcaatccaaaataaggtatattattgatgatg

SEQ ID NO: 29 - protein, 440 aa, Adenoviridae - Mastadenovirus
MSKKRVRVDDDFDPVYPYDADNAPTVPFINPPFVSSDGFQEKPLGVLSLRLADPVTTKNGEITLKLGEGVDLDDSGKLISKNATKATAPLSISNSTISLNMDAPLYNNNGKLGIRIGAPLKVVDLLNTLAVAYGSGLGLKNNALTVQLVSPLTFDNKGNVKINLGNGPLTVAANRLSVTCKRGLYVTTTGDALESNISWAKGIRFEGNAIAANIGKGLEFGTTSSESDVSNAYPIQVKLGTGLTFDSTGAIVAWNKEDDKLTLWTTADPSPNCHIYSDKDAKLTLCLTKCGSQILGTVSLIAVDTGSLNPITGQVTTALVSLKFDANGVLQTSSTLDKEYWNFRKGDVTPAEPYTNAIGFMPNIKAYPKNTNSAAKSHIVGKVYLHGEVSKPLDLIITFNETSNETCTYCINFQWQWGTDKYKNETLAVSSFTFSYIAQE

SEQ ID NO: 30 - protein, 443 aa, Adenoviridae - Mastadenovirus
MSKKRVRVDDDFDPVYPYDADNAPTVPFINPPFVSSDGFQEKPLGVLSLRLADPVTTKNGEITLKLGEGVDLDDSGKLISKNATKATAPLSISNSTISLNMAAPFYNNNGTLSLNVSTPLAVFPTFNTLGISLGNGLQTSNKLLAVQLTHPLTFSSNSITVKTDKGLYINSSGNRGLEANISLKRGLIFDGNAIATYLGSGLDYGSYDSDGKTRPIITKIGAGLNFDSNNAMAVKLGTGLSFDSAGALTAGNKEDDKLTLWTTPDPSPNCQLLSDRDAKFTLCLTKCGSQILGTVAVAAVTVSSALNPINDTVKSAIVFLRFDSDGVLMSNSSMVGDYWNFREGQTTQSVAYTNAVGFMPNLGAYPKTQSKTPKNSIVSQVYLNGETTMPMTLTITFNGTDEKDTTPVSTYSMTFTWQWTGDYKDKNITFATNSFTFSYMAQE

SEQ ID NO: 31 - protein, 425 aa, Adenoviridae - Mastadenovirus
MSKKRVRVDDDFDPVYPYDADNAPTVPFINPPFVSSDGFQEKPLGVLSLRLADPVTTKNGEITLKLGEGVDLDSSGKLISNTATKAAAPLSFSNNTISLNMDHPFYTKDGKLALQVSPPLNILRTSILNTLALGFGSGLGLRGSALAVQLVSPLTFDTDGNIKLTLDRGLHVTTGDAIESNISWAKGLKFEDGAIATNIGNGLEFGSSSTETGVDDAYPIQVKLGSGLSFDSTGAIMAGNKEDDKLTLWTTPDPSPNCQILAENDAKLTLCLTKCGSQILATVSVLVVGSGNLNPITGTVSSAQVFLRFDANGVLLTEHSTLKKYWGYRQGDSIDGTPYVNAVGFMPNLKAYPKSQSSTTKNNIVGQVYMNGDVSKPMLLTITLNGTDDSNSTYSMSFSYTWTNGSYVGATFGANSYTFSYIAQE

SEQ ID NO: 32 - protein, 425 aa, Adenoviridae - Mastadenovirus
MSKKRVRVDDDFDPVYPYDADNAPTVPFINPPFVSSDGFQEKPLGVLSLRLADPVTTKNGEITLKLGEGLDLDSSGKLISNTATKAAAPLSFSNNTISLNMDHPFYTKDGKLSLQVSPPLNILRTSILNTLALGFGSGLGLRGSALAVQLVSPLTFDTDGNIKLTLDRGLHVTTGDAIESNISWAKGLKFEDGAIATNIGNGLEFGSSSTETGVDDAYPIQVKLGSGLSFDSTGAIMAGNKEDDKLTLWTTPDPSPNCQILAENDAKLTLCLTKCGSQILATVSVLVVGSGNLNPITGTVSSAQVFLRFDANGVLLTEHSTLKKYWGYRQGDSIDGTPYTNAVGFMPNLKAYPKSQSSTTKNNIVGQVYMNGDVSKPMLLTITLNGTDDSNSTYSMSFSYTWTNGSYVGATFGANSYTFSYIAQE

SEQ ID NO: 33 - protein, 442 aa, Adenoviridae - Mastadenovirus
MSKKRARVDDGFDPVYPYDADNAPTVPFINPPFVSSDGFQEKPLGVLSLRLADPVTTKNGAVPLKLGEGVDLDDSGKLISKKSTKANSPLSISNNTISLNMDTPFYTKDGKLTMQVTAPLKLANTAILNTLAMAYGNGLGLNNNALTVQVTSPLTFDNSKVKINLGNGPLMVSANKLSINCLRGLYVAPNNTGLETNISWANAMRFEGNAMAVYIDTNKGLQFGTTSTETGVTNAYPIQVKLGAGLAFDSTGAIVAWNKENDSLTLWTTPDPSPNCKIASEKDAKLTLCLTKCGSQILGTVSLLAVSGSLAPITGAVSTALVSLKFNANGALLDKSTLNKEYWNYRQGDLIPGTPYTHAVGFMPNKKAYPKNTTAASKSHIVGDVYLDGDADKPLSLIITFNETDDETCDYCINFQWKWGADQYKDKTLATSSFTFSYIAQE

SEQ ID NO: 34 - protein, 577 aa, Adenoviridae - Mastadenovirus
MKRAKTSDETFNPVYPYDTENGPPSVPFLTPPFVSPDGFQESPPGVLSLRLSEPLVTSHGMLALKMGNGLSLDDAGNLTSQDVTTVTPPLKKTKTNLSLQTSAPLTVSSGSLTVAAAAPLAVAGTSLTMQSQAPLTVQDAKLGLATQGPLTVSEGKLTLQTSAPLTAADSSTLTVSATPPLSTSNGSLSIDMQAPIYTTNGKLALNIGAPLHVVDTLNALTVVTGQGLTINGRALQTRVTGALSYDTEGNIQLQAGGGMRIDNNGQLILNVAYPFDAQNNLSLRLGQGPLIVNSAHNLDLNLNRGLYLFTSGNTKKLEVNIKTAKGLFYDGTAIAINAGDGLQFGSGSDTNPLQTKLGLGLEYDSNKAIITKLGTGLSFDNTGAITVGNKNDDKLTLWTTPDPSPNCRINSEKDAKLTLVLTKCGSQVLASVSVLSVKGSLAPISGTVTSAQIVLRFDENGVLLSNSSLDPQYWNYRKGDSTEGTAYTNAVGFMPNLTAYPKTQSQTAKSNIVSQVYLNGDKTKPMTLTITLNGTNETGDATVSTYSMSFSWNWNGSNYINDTFQTNSFTFSYIAQE

SEQ ID NO: 35 - protein, 937 aa, Adenoviridae - Mastadenovirus
MATPSMLPQWAYMHIAGQDASEYLSPGLVQFARATDTYFSLGNKFRNPTVAPTHDVTTDRSQRLTLRFVPVDREDNTYSYKVRYTLAVGDNRVLDMASTYFDIRGVLDRGPSFKPYSGTAYNSLAPKGAPNTSQWITKDNGTDKTYSFGNAPVRGLDITEEGLQIGPDESGGESKKIFADKTYQPEPQLGDEEWHDTIGAEDKYGGRALKPATNMKPCYGSFAKPTNAKGGQAKSRTKDDGTTEPDIDMAFFDDRSQQASFSPELVLYTENVDLDTPDTHIIYKPGTDETSSSFNLGQQSMPNRPNYIGFRDNFIGLMYYNSTGNMGVLAGQASQLNAVVDLQDRNTELSYQLLLDSLGDRTRYFSMWNQAVDSYDPDVRIIENHGVEDELPNYCFPLNGVGFTDTFQGIKVKTTNNGTANATEWESDTSVNNANEIAKGNPFAMEINIQANLWRNFLYANVALYLPDSYKYTPANITLPTNTNTYDYMNGRVVAPSLVDAYINIGARWSLDPMDNVNPFNHHRNAGLRYRSMLLGNGRYVPFHIQVPQKFFAIKSLLLLPGSYTYEWNFRKDVNMILQSSLGNDLRTDGASIAFTSINLYATFFPMAHNTASTLEAMLRNDTNDQSFNDYLSAANMLYPIPANATNVPISIPSRNWAAFRGWSFTRLKTRETPSLGSGFDPYFVYSGSIPYLDGTFYLNHTFKKVSITFDSSVSWPGNDRLLTPNEFEIKRTVDGEGYNVAQCNMTKDWFLVQMLAHYNIGYQGFYVPEGYKDRMYSFFRNFQPMSRQVVDEVNYKDYQAVTLAYQHNNSGFVGYLAPTMRQGQPYPANYPYPLIGKSAVASVTQKKFLCDRVMWRIPFSSNFMSMGALTDLGQNMLYANSAHALDMNFEVDPMDESTLLYVVFEVFDVVRVHQPHRGVIEAVYLRTPFSAGNATT

SEQ ID NO: 36 - protein, 937 aa, Adenoviridae - Mastadenovirus
MATPSMLPQWAYMHIAGQDASEYLSPGLVQFARATDTYFSLGNKFRNPTVAPTHDVTTDRSQRLTLRFVPVDGEDNTYSYKVRYTLAVGDNRVLDMASTYFDIRGVLDRGPSFKPYSGTAYNALAPKGAPNTSQWITKDNGTDKTYSFGNAPVRGLDITEEGLQIRTDESGGESKKIFADKTYQPEPQLGDEEWHDTIGAEDKYGGRALKPATNMKPCYGSFAKPTNAKGGQAKSRTKDDGTTEPDIDMAFFDDRSQQASFSPELVLYTENVDLDTPDTHIIYKPGTDETSSSFNLGQQSMPNRPNYIGFRDNFIGLMYYNSTGNMGVLAGQASQLNAVVDLQDRNTELSYQLLLDSLGDRTRYFSMWNQAVDSYDPDVRIIENHGVEDELPNYCFPLNGVGFTDTFQGIKVKTTNNGTANATEWESDTSVNNANEIAKGNPFAMEINIQANLWRNFLYANVALYLPDSYKYTPANITLPTNTNTYDYMNGRVVAPSLVDAYINIGARWSLDPMDNVNPFNHHRNAGLRYRSMLLGNGRYVPFHIQVPQKFFAIKSLLLLPGSYTYEWNFRKDVNMILQSSLGNDLRTDGASIAFTSINLYATFFPMAHNTASTLEAMLRNDTNDQSFNDYLSAANMLYPIPANATNVPISIPSRNWAAFRGWSFTRLKTRETPSLGSGFDPYFVYSGSIPYLDGTFYLNHTFKKVSITFDSSVSWPGNDRLLTPNEFEIKRTVDGEGYNVAQCNMTKDWFLVQMLAHYNIGYQGFYVPEGYKDRMYSFFRNFQPMSRQVVDEVNYKDYQAVTLAYQHNNSGFVGYLAPTMRQGQPYPANYPYPLIGKSAVASVTQKKFLCDRVMWRIPFSSNFMSMGALTDLGQNMLYANSAHALDMNFEVDPMDESTLLYVVFEVFDVVRVHQPHRGVIKAVYLRTPFSAGNATT

SEQ ID NO: 37 - protein, 937 aa, Adenoviridae - Mastadenovirus
MATPSMLPQWAYMHIAGQDASEYLSPGLVQFARATDTYFSLGNKFRNPTVAPTHDVTTDRSQRLTLRFVPVDREDNTYSYKVRYTLAVGDNRVLDMASTYFDIRGVLDRGPSFKPYSGTAYNSLAPKGAPNTSQWITKDNGTDKTYSFGNAPVRGLDITEEGLQIGTDESGGESKKIFADKTYQPEPQLGDEEWHDTIGAEDKYGGRALKPATNMKPCYGSFAKPTNAKGGQAKSRTKDDGTTEPDIDMAFFDDRSQQASFSPELVLYTENVDLDTPDTHIIYKPGTDETSSSFNLGQQSMPNRPNYIGFRDNFIGLMYYNSTGNMGVLAGQASQLNAVVDLQDRNTELSYQLLLDSLGDRTRYFSMWNQAVDSYDPDVRIIENHGVEDELPNYCFPLNGVGFTDTFQGIKVKTTNNGTANATEWESDTSVNNANEIAKGNPFAMEINIQANLWRNFLYANVALYLPDSYKYTPANITLPTNTNTYDYMNGRVVAPSLVDAYINIGARWSLDPMDNVNPFNHHRNAGLRYRSMLLGNGRYVPFHIQVPQKFFAIKSLLLLPGSYTYEWNFRKDVNMILQSSLGNDLRTDGASIAFTSINLYATFFPMAHNTASTLEAMLRNDTNDQSFNDYLSAANMLYPIPANATNVPISIPSRNWAAFRGWSFTRLKTRETPSLGSGFDPYFVYSGSIPYLDGTFYLNHTFKKVSITFDSSVSWPGNDRLLTPNEFEIKRTVDGEGYNVAQCNMTKDWFLVQMLAHYNIGYQGFYVPEGYKDRMYSFFRNFQPMSRQVVDEVNYKDYQAVTLAYQHNNSGFVGYLAPTMRQGQPYPANYPYPLIGKSAVASVTQKKFLCDRVMWRIPFSSNFMSMGALTDLGQNMLYANSAHALDMNFEVDPMDESTLLYVVFEVFDVVRVHQPHRGVIEAVYLRTPFSAGNATT

SEQ ID NO: 38 - protein, 937 aa, Adenoviridae - Mastadenovirus
  misc_feature (296)..(296): Xaa can be any naturally occurring amino acid
MATPSMLPQWAYMHIAGQDASEYLSPGLVQFARATDTYFSLGNKFRNPTVAPTHDVTTDRSQRLTLRFVPVDREDNTYSYKVRYTLAVGDNRVLDMASTYFDIRGVLDRGPSFKPYSGTAYNSLAPKGAPNTSQWITKDNGTDKTYSFGNAPVRGLDITEEGLQIGTDESGGKSKKIFADKTYQPEPQLGDEEWHDTIGAEDKYGGRALKPATNMKPCYGSFAKPTNAKGGQAKSRTKDDGTTEPDIDMAFFDDRSQQASFSPELVLYTENVDLDTPDTHIIYKPGTDETSSSFNXGQQSMPNRPNYIGFRDNFIGLMYYNSTGNMGVLAGQASQLNAVVDLQDRNTELSYQLLLDSLGDRTRYFSMWNQAVDSYDPDVRIIENHGVEDELPNYCFPLNGVGFTDTFQGIKVKTTNNGTANATEWESDTSVNNANEIAKGNPFAMEINIQANLWRNFLYANVALYLPDSYKYTPANITLPTNTNTYDYMNGRVVAPSLVDAYINIGARWSLDPMDNVNPFNHHRNAGLRYRSMLLGNGRYVPFHIQVPQKFFAIKNLLLLPGSYTYEWNFRKDVNMILQSSLGNDLRTDGASIAFTSINLYATFFPMAHNTASTLEAMLRNDTNDQSFNDYLSAANMLYPIPANATNVPISIPSRNWAAFRGWSFTRLKTRETPSLGSGFDPYFVYSGSIPYLDGTFYLNHTFKKVSITFDSSVSWPGNDRLLTPNEFEIKRTVDGEGYNVAQCNMTKDWFLVQMLAHYNIGYQGFYVPEGYKDRMYSFFRNFQPMSRQVVDEVNYKDYQAVTLAYQHNNSGFVGYLAPTMRQGQPYPANYPYPLIGKSAVASVTQKKFLCDRVMWRIPFSSNFMSMGALTDLGQNMLYANSAHALDMNFEVDPMDESTLLYVVFEVFDVVRVHQPHRGVIEAVYLRTPFSAGNATT

SEQ ID NO: 39 - protein, 937 aa, Adenoviridae - Mastadenovirus
  misc_feature (538)..(538): Xaa can be any naturally occurring amino acid
MATPSMLPQWAYMHIAGQDASEYLSPGLVQFARATDTYFSLGNKFRNPTVAPTHDVTTDRSQRLTLRFVPVDREDNTYSYKVRYTLAVGDNRVLDMASTYFDIRGVLDRGPSFKPYSGTAYNSLAPKGAPNTSQWVTKDNGTDKTYSFGNAPVRGLDITEEGLQIGTDDSSTESKKIFADKTYQPEPQVGDEEWHDTIGAEDKYGGRALKPATNMKPCYGSFAKPTNAKGGQAKTRTKDDGTTEPDIDMAFFDDRSQQASFSPELVLYTENVDLETPDTHIIYKPGTDETSSSFNLGQQSMPNRPNYIGFRDNFIGLMYYNSTGNMGVLAGQASQLNAVVDLQDRNTELSYQLLLDSLGDRTRYFSMWNQAVDSYDPDVRIIENHGVEDELPNYCFPLNGVGFTDTFQGIKVKTTNNGTANATEWESDTSVNNANEIAKGNPFAMEINIQANLWRNFLYANVALYLPDSYKYTPANVTLPTNTNTYEYMNGRVVAPSLVDSYINIGARWSLDPMDNVNPFNHHRNAGLRYRSMLLGNXRFVPFHIQVPQKFFAIKSLLLLPGSYTYEWNFRKDVNMILQSSLGNDLRTDGASISFTSINLYATFFPMAHNTASTLEAMLRNDTNDQSFNDYLSAANMLYPIPANATNVPISIPSRNWAAFRGWSFTRLKTKETPSLGSGFDPYFVYSGSIPYLDGTFYLNHTFKKVSITFDSSVSWPGNDRLLTPNEFEIKRTVDGEGYNVAQCNMTKDWFLVQMLAHYNIGYQGFYVPEGYKDRMYSFFRNFQPMSRQVVDEVNYKDYQAVTLAYQHNNSGFVGYLAPTMRQGQPYPANYPYPLIGKSAVTSVTQKKFLCDRVMWRIPFSSNFMSMGALTDLGQNMLYANSAHALDMNFEVDPMDESTLLYVVFEVFDVVRVHQPHRGVIEAVYLRTPFSAGNATT

SEQ ID NO: 40 - protein, 962 aa, Adenoviridae - Mastadenovirus
MATPSMMPQWSYMHISGQDASEYLSPGLVQFARATDSYFSLSNKFRNPTVAPTHDVTTDRSQRLTLRFIPVDREDTAYSYKARFTLAVGDNRVLDMASTYFDIRGVLDRGPTFKPYSGTAYNSLAPKGAPNSCEWEQVEPAEEAAENEDEEEEEDVVDPQEQEPTTKTHVYAQAPLSGEKITKDGLQIGTEATAAGGTKDLFADPTFQPEPQVGESQWNEADATAAGGRVLKKTTPMKPCYGSYARPTNANGGQGVLKANAQGVLESQVEMQFFSTSTNATNEQNNIQPKLVLYSEDVHMETPDTHISYKPTKSDDNSKVMLGQQSMPNRPNYIAFRDNFIGLMYYNSTGNMGVLAGQASQLNAVVDLQDRNTELSYQLLLDSMGDRTRYFSMWNQAVDSYDPDVRIIENHGTEDELPNYCFPLGGIGITDTYQAIKTNGNGAGDQATTWQKDSQFADRNEIGVGNNFAMEINLSANLWRNFLYSNVALYLPDKLKYNPSNVEISDNPNTYDYMNKRVVAPGLVDCYINLGARWSLDYMDNVNPFNHHRNAGLRYRSMLLGNGRYVPFHIQVPQKFFAIKNLLLLPGSYTYEWNFRKDVNMVLQSSLGNDLRVDGASIKFESICLYATFFPMAHNTASTLEAMLRNDTNDQSFNDYLSAANMLYPIPANATNVPISIPSRNWAAFRGWAFTRLKTKETPSLGSGFDPYYTYSGSIPYLDGTFYLNHTFKKVSVTFDSSVSWPGNDRLLTPNEFEIKRSVDGEGYNVAQCNMTKDWFLIQMLANYNIGYQGFYIPESYKDRMYSFFRNFQPMSRQVVDETKYKDYQQVGIIHQHNNSGFVGYLAPTMREGQAYPANFPYPLIGKTAVDSITQKKFLCDRTLWRIPFSSNFMSMGALTDLGQNLLYANSAHALDMTFEVDPMDEPTLLYVLFEVFDVVRVHQPHRGVIETVYLRTPFSAGNATT

SEQ ID NO: 41 - protein, 531 aa, Adenoviridae - Mastadenovirus
MMRRVYPEGPPPSYESVMQQAVAAAMQPPLEAPYVPPRYLAPTEGRNSIRYSELAPLYDTTRLYLVDNKSADIASLNYQNDHSNFLTTVVQNNDFTPTEASTQTINFDERSRWGGQLKTIMHTNMPNVNEFMYSNKFKARVMVSRKTPNGVAVGDDYDGGQDELTYEWVEFELPEGNFSVTMTIDLMNNAIIDNYLAVGRQNGVLESDIGVKFDTRNFRLGWDPVTELVMPGVYTNEAFHPDIVLLPGCGVDFTESRLSNLLGIRKRQPFQEGFQILYEDLEGGNIPALLDVEAYEKSKEESAAAATAAVATASTEVRGDNFASAAAVAEAAETESKIVIQPVEKDSKDRSYNVLADKKNTAYRSWYLAYNYGDPEKGVRSWTLLTTSDVTCGVEQVYWSLPDMMQDPVTFRSTRQVSNYPVVGAELLPVYSKSFFNEQAVYSQQLRAFTSLTHVFNRFPENQILVRPPAPTITTVSENVPALTDHGTLPLRSSIRGVQRVTVTDARRRTCPYVYKALGVVAPRVLSSRTF

SEQ ID NO: 42 - protein, 541 aa, Adenoviridae - Mastadenovirus
MMRRVYPEGPPPSYESVMQQAVAVAMQPPLEAPYVPPRYLAPTEGRNSIRYSELAPLYDTTRLYLVDNKSADIASLNYQNDHSNFLTTVVQNNDFTPTEASTQTINFDERSRWGGQLKTIMHTNMPNVNEFMYSNKFKARVMVSRKTPNGVTVGDDYDGSQDELTYEWVEFELPEGNFSVTMTIDLMNNAIIDNYLAVGRQNGVLESDIGVKFDTRNFRLGWDPVTELVMPGVYTNEAFHPDIVLLPGCGVDFTESRLSNLLGIRKRQPFQEGFQILYEDLEGGNIPALLDVEAYEKSKEDSAAATTAAVATAATTDADATTTRGDTFATQAEEAAALAATDDSESKIVIKPVEKDSKDRSYNVLADKKNTAYRSWYLAYNYGDPEKGVRSWTLLTTSDVTCGVEQVYWSLPDMMQDPVTFRSTRQVSNYPVVGAELLPVYSKSFFNEQAVYSQQLRAFTSLTHVFNRFPENQILVRPPAPTITTVSENVPALTDHGTLPLRSSIRGVQRVTVTDARRRTCPYVYKALGVVAPRVLSSRTF

SEQ ID NO: 43 - protein, 532 aa, Adenoviridae - Mastadenovirus
MMRRVYPEGPPPSYESVMQQAVAAAMQPPLEAPYVPPRYLAPTEGRNSIRYSELAPLYDTTRLYLVDNKSADIASLNYQNDHSNFLTTVVQNNDFTPTEASTQTINFDERSRWGGQLKTIMHTNMPNVNEFMYSNKFKARVMVSRKTPNGVTVTDGSQDELTYEWVEFELPEGNFSVTMTIDLMNNAIIDNYLAVGRQNGVLESDIGVKFDTRNFRLGWDPVTELVMPGVYTNEAFHPDIVLLPGCGVDFTESRLSNLLGIRKRQPFQEGFQILYEDLEGGNIPALLDVEAYEKSKEDSTAVATAATVADATVTRGDTFATQAEEAAALAATDDSESKIVIKPVEKDSKDRSYNVLSDGKNTAYRSWYLAYNYGDPEKGVRSWTLLTTSDVTCGVEQVYWSLPDMMQDPVTFRSTRQVSNYPVVGAELLPVYSKSFFNEQAVYSQQLRAFTSLTHVFNRFPENQILVRPPAPTITTVSENVPALTDHGTLPLRSSIRGVQRVTVTDARRRTCPYVYKALGVVAPRVLSSRTF

SEQ ID NO: 44 - protein, 528 aa, Adenoviridae - Mastadenovirus
MMRRVYPEGPPPSYESVMQQAVAAAMQPPLEAPYVPPRYLAPTEGRNSIRYSELAPLYDTTRLYLVDNKSADIASLNYQNDHSNFLTTVVQNNDFTPTEASTQTINFDERSRWGGQLKTIMHTNMPNVNEFLYSNKFKARVMVSRKTPNGVTVTDGSQDELTYEWVEFELPEGNFSVTMTIDLMNNAIIDNYLAVGRQNGVLESDIGVKFDTRNFRLGWDPVTELVMPGVYTNEAFHPDIVLLPGCGVDFTESRLSNLLGIRKRQPFQEGFQIMYEDLEGGNIPALLDVEAYEKSKEDSAAAATAAVATASTEVRGDNFASAAAVAEAAETESKIVIQPVEKDSKDRSYNVLADKKNTAYRSWYLAYNYGDPEKGVRSWTLLTTSDVTCGVEQVYWSLPDMMQDPVTFRSTRQVSNYPVVGAELLPVYSKSFFNEQAVYSQQLRAFTSLTHVFNRFPENQILVRPPAPTITTVSENVPALTDHGTLPLRSSIRGVQRVTVTDARRRTCPYVYKALGVVAPRVLSSRTF

SEQ ID NO: 45 - protein, 535 aa, Adenoviridae - Mastadenovirus
MMRRAYPEGPPPSYESVMQQAMAAAAAMQPPLEAPYVPPRYLAPTEGRNSIRYSELAPLYDTTRLYLVDNKSADIASLNYQNDHSNFLTTVVQNNDFTPTEASTQTINFDERSRWGGQLKTIMHTNMPNVNEFMYSNKFKARVMVSRKTPNGVTVTEDYDGSQDELKYEWVEFELPEGNFSVTMTIDLMNNAIIDNYLAVGRQNGVLESDIGVKFDTRNFRLGWDPVTELVMPGVYTNEAFHPDIVLLPGCGVDFTESRLSNLLGIRKRQPFQEGFQIMYEDLEGGNIPALLDVDAYEKSKEESAAAATAAVATASTEVRGDNFASAAAVAAAEAAETESKIVIQPVEKDSKDRSYNVLPDKINTAYRSWYLAYNYGDPEKGVRSWTLLTTSDVTCGVEQVYWSLPDMMQDPVTFRSTRQVSNYPVVGAELLPVYSKSFFNEQAVYSQQLRAFTSLTHVFNRFPENQILVRPPAPTITTVSENVPALTDHGTLPLRSSIRGVQRVTVTDARRRTCPYVYKALGIVAPRVLSSRTF

SEQ ID NO: 46 - protein, 581 aa, Adenoviridae - Mastadenovirus
MRRAAMYHEGPPPSYESVVGAAAASPFASQLEPPYVPPRYLRPTGGRNSIRYSELAPLYDTTRVYLVDNKSADVASLNYQNDHSNFLTTVIQNNDYTPSEASTQTINLDDRSHWGGDLKTILHTNMPNVNEFMFTNKFKARVMVSRSHTKDDRVELKYEWVEFELPEGNYSETMTIDLMNNAIVEHYLKVGRQNGVLESDIGVKFDTRNFRLGLDPVTGLVMPGVYTNEAFHPDIILLPGCGVDFTYSRLSNLLGIRKRQPFQEGFRITYEDLEGGNIPALLDVEAYQDSLKEEEAGEGSGGGAGQEEGGASSEASADPAAAAEAEAADPAMVVEEEKDMNDEAVRGDTFATRGEEKKAEAEAAAEEAAAAAAAVEAAAEAEKPPKEPVIKPLTEDSKKRSYNVLKDSTNTEYRSWYLAYNYGDPATGVRSWTLLCTPDVTCGSEQVYWSLPDMMQDPVTFRSTRQVSNFPVVGAELLPVHSKSFYNDQAVYSQLIRQFTSLTHVFNRFPENQILARPPAPTITTVSENVPALTDHGTLPLRNSIGGVQRVTVTDARRRTCPYVYKALGIVSPRVLSSRTF

SEQ ID NO: 47 - protein, 577 aa, Adenoviridae - Mastadenovirus; identical to SEQ ID NO: 34

SEQ ID NO: 48 - protein, 955 aa, Adenoviridae - Mastadenovirus
MATPSMMPQWSYMHISGQDASEYLSPGLVQFARATDSYFSLSNKFRNPTVAPTHDVTTDRSQRLTLRFIPVDREDTAYSYKARFTLAVGDNRVLDMASTYFDIRGVLDRGPTFKPYSGTAYNSLAPKGAPNPCEWDEAVTAVDINLDELGEDEDDAEGEAEQQKSHVFGQAPYSGQNITKEGIQIGVDTTSQAQTPLYADKTFQPEPQVGESQWNETEINYGAGRVLKKTTLMKPCYGSYARPTNENGGQGILLEKEGGKPESQVEMQFFSTTQAAAAGNSDNLTPKVVLYSEDVHLETPDTHISYMPTSNEANSRELLGQQAMPNRPNYIAFRDNFIGLMYYNSTGNMGVLAGQASQLNAVVDLQDRNTELSYQLLLDSMGDRTRYFSMWNQAVDSYDPDVRIIENHGTEDELPNYCFPLGGIINTETLTKVKPKTGQDAQWEKDTEFSEKNEIRVGNNFAMEINLNANLWRNFLYSNVALYLPDKLKYTPANVQISSNSNSYDYMNKRVVAPGLVDCYINLGARWSLDYMDNVNPFNHHRNAGLRYRSMLLGNGRYVPFHIQVPQKFFAIKNLLLLPGSYTYEWNFRKDVNMVLQSSLGNDLRVDGASIKFESICLYATFFPMAHNTASTLEAMLRNDTNDQSFNDYLSAANMLYPIPANATNVPISIPSRNWAAFRGWAFTRLKTKETPSLGSGFDPYYTYSGSIPYLDGTFYLNHTFKKVSVTFDSSVSWPGNDRLLTPNEFEIKRSVDGEGYNVAQCNMTKDWFLIQMLANYNIGYQGFYIPESYKDRMYSFFRNFQPMSRQVVDETKYKDYQQVGIIHQHNNSGFVGYLAPTMREGQAYPANFPYPLIGKTAVDSVTQKKFLCDRTLWRIPFSSNFMSMGALTDLGQNLLYANSAHALDMTFEVDPMDEPTLLYVLFEVFDVVRVHQPHRGVIETVYLRTPFSAGNATT

SEQ ID NO: 49 - protein, 582 aa, Adenoviridae - Mastadenovirus
MRRAAMYHEGPPPSYESVVGAAAASPFASQLEPPYVPPRYLRPTGGRNSIRYSELAPLYDTTRVYLVDNKSADVASLNYQNDHSNFLTTVIQNNDYTPSEASTQTINLDDRSHWGGDLKTILHTNMPNVNEFMFTNKFKARVMVSRSHTKDDRVELKYEWVEFELPEGNYSETMTIDLMNNAIVEHYLKVGRQNGVLESDIGVKFDTRNFRLGLDPVTGLVMPGVYTNEAFHPDIILLPGCGVDFTYSRLSNLLGIRKRQPFQEGFRITYEDLEGGNIPALLDVEAYQNSLKEEEAGEGSGGGGAGQEEGGASSEASADAAAAEAEEAADPAMVVEEEKDMNDEAVRGDTFATRGEEKKAEAEAAAEEAAAAAAAVEAAAEAEKPPKEPVIKPLTEDSKKRSYNVLKDSTNTEYRSWYLAYNYGDPATGVRSWTLLCTPDVTCGSEQVYWSLPDMMQDPVTFRSTRQVSNFPVVGAELLPVHSKSFYNDQAVYSQLIRQFTSLTHVFNRFPENQILARPPAPTITTVSENVPALTDHGTLPLRNSIGGVQRVTVTDARRRTCPYVYKALGIVSPRVLSSRTF

SEQ ID NO: 50 - protein, 542 aa, Adenoviridae - Mastadenovirus
MKRAKTSDETFNPVYPYDTENGPPSVPFLTPPFVSPDGFQESPPGVLSLRLSEPLVTSHGMLALKMGNGLSLDDAGNLTSQDVTTVTPPLKKTKTNLSLQTSAPLTVSSGSLTVAAAAPLAVAGTSLTMQSQAPLTVQDAKLGLATQGPLTVSEGKLTLQTSAPLTAADSSTLTVGTTPPISVSSGSLGLDMEDPMYTHDGKLGIRIGGPLQVVDSLHTLTVVTGNGITVANNALQTKVAGALGYDSSGNLELRAAGGMRINTGGQLILDVAYPFDAQNNLSLRLGQGPLYVNTNHNLDLNCNRGLTTTTSSNTTKLETKIDSGLDYNANGAIIAKLGTGLTFDNTGAITVGNTGDDKLTLWTTPDPSPNCRIHADKDCKFTLVLTKCGSQILASVAALAVSGNLSSMTGTVSSVTIFLRFDQNGVLMENSSLDKEYWNFRNGNSTNATPYTNAVGFMPNLSAYPKTQSQTAKNNIVSEVYLHGDKSKPMILTITLNGTNESSETSQVSHYSMSFTWSWDSGKYATETFATNSFTFSYIAEQ

SEQ ID NO: 51 - protein, 964 aa, Adenoviridae - Mastadenovirus
MATPSMMPQWSYMHISGQDASEYLSPGLVQFARATDSYFSLSNKFRNPTVAPTHDVTTDRSQRLTLRFIPVDREDTAYSYKARFTLAVGDNRVLDMASTYFDIRGVLDRGPTFKPYSGTAYNSLAPKGAPNSCEWEQEETQTAEEAQDEEEDEAEAEEEMPQEEQAPVKKTHVYAQAPLSGEKITKDGLQIGTDATATEQKPIYADPTFQPEPQIGESQWNEADASVAGGRVLKKTTPMKPCYGSYARPTNANGGQGVLVEKDGGKMESQVDMQFFSTSENARNEANNIQPKLVLYSEDVHMETPDTHISYKPAKSDDNSKVMLGQQSMPNRPNYIGFRDNFIGLMYYNSTGNMGVLAGQASQLNAVVDLQDRNTELSYQLLLDSMGDRTRYFSMWNQAVDSYDPDVRIIENHGTEDELPNYCFPLGGIGVTDTYQAIKTNGNGNGGGNTTWTKDETFADRNEIGVGNNFAMEINLSANLWRNFLYSNVALYLPDKLKYNPSNVEISDNPNTYDYMNKRVVAPGLVDCYINLGARWSLDYMDNVNPFNHHRNAGLRYRSMLLGNGRYVPFHIQVPQKFFAIKNLLLLPGSYTYEWNFRKDVNMVLQSSLGNDLRVDGASIKFESICLYATFFPMAHNTASTLEAMLRNDTNDQSFNDYLSAANMLYPIPANATNVPISIPSRNWAAFRGWAFTRLKTKETPSLGSGFDPYYTYSGSIPYLDGTFYLNHTFKKVSVTFDSSVSWPGNDRLLTPNEFEIKRSVDGEGYNVAQCNMTKDWFLIQMLANYNIGYQGFYIPESYKDRMYSFFRNFQPMSRQVVDETKYKDYQQVGIIHQHNNSGFVGYLAPTMREGQAYPANFPYPLIGKTAVDSVTQKKFLCDRTLWRIPFSSNFMSMGALTDLGQNLLYANSAHALDMTFEVDPMDEPTLLYVLFEVFDVVRVHQPHRGVIETVYLRTPFSAGNATT

SEQ ID NO: 52 - protein, 584 aa, Adenoviridae - Mastadenovirus
MRRAAMYHEGPPPSYESVVGAAAASPFASQLEPPYVPPRYLRPTGGRNSIRYSELAPLYDTTRVYLVDNKSADVASLNYQNDHSNFLTTVIQNNDYTPSEASTQTINLDDRSHWGGDLKTILHTNMPNVNEFMFTNKFKARVMVSRSHTKDDRVELKYEWVEFELPEGNYSETMTIDLMNNAIVEHYLKVGRQNGVLESDIGVKFDTRNFRLGLDPVTGLVMPGVYTNEAFHPDIILLPGCGVDFTYSRLSNLLGIRKRQPFQEGFRITYEDLEGGNIPALLDVEAYQDSLKEEEAGEGSGGGGGAGQEEGGASSEASADAAAAAEAEAADPAMVVEEEKDMNDEAVRGDTFATRGEEKKAEAEAAAEEAAAAAAAAVEAAAEAEKPPKEPVIKALTEDSKKRSYNVLKDSTNTAYRSWYLAYNYGDPATGVRSWTLLCTPDVTCGSEQVYWSLPDMMQDPVTFRSTRQVSNFPVVGAELLPVHSKSFYNDQAVYSQLIRQFTSLTHVFNRFPENQILARPPAPTITTVSENVPALTDHGTLPLRNSIGGVQRVTVTDARRRTCPYVYKALGIVSPRVLSSRTF

(19) United States
(12) Patent Application Publication
(10) Pub. No.: US 2011/0129498 A1
(43) Pub. Date: Jun. 2, 2011

(54) VACCINE FOR THE PREVENTION AND THERAPY OF HCV INFECTIONS

(75) Inventors: Riccardo Cortese, Roma (IT); Alfredo Nicosia, Roma (IT)

(73) Assignee: OKAIROS AG, Basel (CH)

(21) Appl. No.: 12/995,807

(22) PCT Filed: Jun. 3, 2009
(86) PCT No.: PCT/EP2009/003970

§ 371 Date: Feb. 7, 2011

Related U.S. Application Data
(60) Provisional application No. 61/058,356, filed on Jun. 3, 2008.

(51) Int. Cl. A61K 39/29; C07K 14/18; C07H 21/00; C12N 15/63; A61P 31/14; A61P 37/04

(52) U.S. Cl. 424/228.1; 530/350; 536/23.72; 435/320.1; 530/300

(57) ABSTRACT

The present invention relates to CD81-binding peptides of the hepatitis virus C(HCV) E2 glycoprotein, which are devoid of or mutated within the amino-terminal 27 amino acids of the mature E2 envelope glycoprotein, or variant thereof which retains the ability to bind to CD81. Furthermore, the present invention provides polypeptides comprising said CD81-binding peptide, polynucleotides encoding the CD81-binding peptide, and expression cassettes and vectors comprising the polynucleotide of the invention. Moreover, the present invention relates to compositions comprising the CD81-binding peptide, the polynucleotide encoding the CD81-binding peptide, the expression cassette, or the vector, and an adjuvant. Furthermore, the present invention provides a pharmaceutical composition comprising the CD81-binding peptide, the polynucleotide, the expression cassette, the vector, or the composition of the invention, and a pharmaceutically acceptable excipient, carrier, or diluent. Moreover, the present invention provides the CD81-binding peptide, the polynucleotide, the expression cassette, the vector, the composition, or the pharmaceutical composition of the invention for induction of an immune response, preferably a broad specificity immune response, against HCV in a mammal, and methods for inducing a therapeutic and/or prophylactic immune response against HCV in a mammal, preferably against HCV of various genotypes.

### TECHNICAL FIELD OF INVENTION

The present invention relates to CD81-binding peptides derived from the Hepatitis C virus (HCV) glycoprotein E2 devoid of or mutated within the hypervariable region 1 (HVR1) that are capable of inducing a broad-specificity prophylactic and/or therapeutic immune response against infection with various HCV genotypes and their use.

### BACKGROUND OF THE INVENTION

Approximately 3% of the world population (around 170 million people) are infected with the hepatitis C virus (HCV), and about 50 to 80% of the acute infected subjects develop chronic hepatitis with viral persistence being at risk of developing liver cirrhosis and hepatocellular carcinoma (Timm and Roggendorf, 2007).

HCV is a member of the family of Flaviviridae, with a 9.5 kb positive-strand RNA genome that encodes three structural proteins, the capsid and viral envelope proteins E1 and E2, and at least six nonstructural proteins, NS2, NS3, NS4A, NS4B, NS5A, and NS5B. The entire genome is translated into a protein of about 3000 amino acids, which is later cut into the separate proteins by cellular and viral proteases. The amino acid coordinates of the HCV structural and nonstructural proteins within the HCV polyprotein are approximately as follows: capsid (aa 1 to 191), E1 (aa 192 to 383), E2 (384 to 746), P7 (aa 747 to 809), NS2 (aa 810 to 1026), NS3 (aa 1027 to 1657), NS4A (aa 1658 to 1711), NS4B (aa 1712 to 1972), NS5A (aa 1973 to 2420), and NS5B (aa 2421 to 3011), wherein the amino acid positions may be shifted by a few amino acids depending on the HCV genotype or isolate. The HCV particle consists of a nucleocapsid surrounded by a lipid bilayer in which the two envelope glycoproteins, E1 and E2, are anchored as a heterodimer (Lavie et al., 2007). HCV E2 is a ˜70 kDa glycoprotein that shows large variation among HCV genotypes and contains a 27 amino acid sequence at its amino terminus that is highly variable and is designated the hypervariable region 1 (HVR1). The envelope proteins are thought to be the primary mediators of virion attachment and cell entry. An essential step during the infection by HCV is the molecular interaction of its envelope glycoprotein E2 (or heterodimeric E1 E2 complex) with a series of cellular membrane proteins (HCV receptors). Initial attachment of the virion may involve glycosaminoglycans and the low density lipoprotein receptor, and it is followed by the sequential interaction with the scavenger receptor class B type 1, the tetraspanning CD81 and tight junction protein Claudin-1, -6, or -9 (Dubuisson et al., 2008). It has been described that interference of the interaction with HCV envelope proteins and CD81 by anti-CD81 antibodies inhibits or interferes with HCV infection (Keck et al., 2008).

Based on phylogenetic analysis, a classification of HCV into six major genotypes was proposed and criteria for the designation of new HCV variants were formulated (Timm and Roggendorf, 2007). These proposals provide an HCV nomenclature scheme for the three major public HCV sequence data bases: Europe (Combet et al., 2007), USA (Kuiken et al., 2005), and Japan (http://s2as02.genes.nig.ac.jp/). HCV genotypes differ from each other by 31%-33% on the nucleotide level, and the genotypes are further divided into multiple epidemiologically distinct subtypes differing by 20% to 25% from one another (Simmonds, 2004).

The virally encoded RNA Polymerase of HCV lacks proof reading function, and thus, the replication of the viral genome is error prone. Theoretically, every possible mutation in every single position of the genome will be generated in one infected host every day (Timm & Roggendorf, 2007). This high error rate is reflected in the generation of a heterogeneous, but closely related swarm of viruses within the same host referred to as quasispecies (Simmonds, 2004). It is believed that the high level of genetic variability enables the HCV to escape the immune system and usually leads to chronic disease. One of the most variable regions is hypervariable region 1 (HVR1) located to the 27 amino terminal amino acids within the envelope glycoprotein E2. Antibodies directed at HCV envelope determinants responsible for receptor recognition are deemed important for neutralization of HCV infections. However, those antibodies targeting the HVR1 have been shown to be isolate-specific. Consistently, immunogens including HCV glycoproteins comprising full length or carboxy terminally truncated versions of E2 or the dimeric complex E1E2, all bearing the HVR1, are capable of inducing neutralizing antibodies that are efficacious in preventing infection from homologous HCV strains, but are much less effective in the prevention of infection with heterologous HCV strains, and are therefore not suitable for broad-specificity protection against HCV infection by vaccination.

The present invention provides antigens/immunogens that are capable of eliciting an immune response against heterologous HCV strains, and thus, these antigens are applicable for broad-specificity protection by vaccination or therapy against HCV infection.

### SUMMARY OF THE INVENTION

In a first aspect, the present invention relates to a CD81-binding peptide of HCV E2, which is devoid of or mutated within the N-terminal 27 amino acids of the mature E2 envelope glycoprotein, or variant thereof which retains the ability to bind to CD81,

a polypeptide comprising said peptide, with the proviso that said polypeptide is not a wild type E2,
a polynucleotide encoding said peptide or said polypeptide, an expression cassette comprising (i) said polynucleotide, and (ii) one or more polynucleotides selected from the group consisting of poly-adenylation signal, promoter, enhancer, a nucleotide sequence encoding a heterologous protein, and a nucleotide sequence encoding a peptide-tag,
a vector comprising said polynucleotide or said expression cassette,
a composition comprising said peptide, said polypeptide, said polynucleotide, said expression cassette, or said vector, and an adjuvant, or
a pharmaceutical composition comprising said peptide, said polypeptide, said polynucleotide, said expression cassette, said vector, or said composition, and a pharmaceutically acceptable excipient, carrier, or diluent,
for induction of an immune response against HCV in a mammal.

In another embodiment, the present invention relates to a method for inducing an immune response in a mammal against HCV comprising administering to said mammal

a CD81-binding peptide of HCV E2, which is devoid of or mutated within the N-terminal 27 amino acids of the mature E2 envelope glycoprotein, or variant thereof which retains the ability to bind to CD81,
a polypeptide comprising said peptide, with the proviso that said polypeptide is not a wild type E2,
a polynucleotide encoding said peptide or said polypeptide,
an expression cassette comprising (i) said polynucleotide, and (ii) one or more polynucleotides selected from the group consisting of poly-adenylation signal, promoter, enhancer, a nucleotide sequence encoding a heterologous protein, and a nucleotide sequence encoding a peptide-tag,
a vector comprising said polynucleotide or said expression cassette,
a composition comprising said peptide, said polypeptide, said polynucleotide, said expression cassette, or said vector, and an adjuvant, or
a pharmaceutical composition comprising said peptide, said polypeptide, said polynucleotide, said expression cassette, said vector, or said composition, and a pharmaceutically acceptable excipient, carrier, or diluent,
in an amount effective to generate an immune response.

In a preferred embodiment, the immune response is therapeutic and/or prophylactic. Preferably, the immune response is directed against two or more different HCV genotypes.

In a preferred embodiment, the vector for generating the immune response (priming) is selected from the group consisting of DNA plasmid, adenovirus (Ad) vectors (e.g., non-replicating Ad5, Ad11, Ad26, Ad35, Ad49, ChAd3, ChAd4, ChAd5, ChAd6, ChAd7, ChAd8, ChAd9, ChAd10, ChAd11, ChAd16, ChAd17, ChAd19, ChAd20, ChAd22, ChAd24, ChAd26, ChAd30, ChAd31, ChAd37, ChAd38, ChAd44, ChAd63, ChAd82, ChAd55, ChAd73, ChAd83, ChAd146, ChAd147, PanAd1, PanAd2, and PanAd3 vectors or replication-competent Ad4 and Ad7 vectors), adeno-associated virus (AAV) vectors (e.g., AAV type 5), alphavirus vectors (e.g., Venezuelan equine encephalitis virus (VEE), sindbis virus (SIN), semliki forest virus (SFV), and VEE-SIN chimeras), herpes virus vectors, measles virus vectors, pox virus vectors (e.g., vaccinia virus, modified vaccinia virus Ankara (MVA), NYVAC (derived from the Copenhagen strain of vaccinia), and avipox vectors: canarypox (ALVAC) and fowlpox (FPV) vectors), and vesicular stomatitis virus vectors, and the vector for enhancing the immune response (boosting) is selected from the group consisting of DNA plasmid, adenovirus (Ad) vectors (e.g., non-replicating Ad5, Ad11, Ad26, Ad35, Ad49, ChAd3, ChAd4, ChAd5, ChAd6, ChAd7, ChAd8, ChAd9, ChAd10, ChAd11, ChAd16, ChAd17, ChAd19, ChAd20, ChAd22, ChAd24, ChAd26, ChAd30, ChAd31, ChAd37, ChAd38, ChAd44, ChAd63, ChAd82, ChAd55, ChAd73, ChAd83, ChAd146, ChAd147 PanAd1, PanAd2, and PanAd3 vectors or replication-competent Ad4 and Ad7 vectors), adeno-associated virus (AAV) vectors (e.g., AAV type 5), alphavirus vectors (e.g., Venezuelan equine encephalitis virus (VEE), sindbis virus (SIN), semliki forest virus (SFV), and VEE-SIN chimeras), herpes virus vectors, measles virus vectors, pox virus vectors (e.g., vaccinia virus, modified vaccinia virus Ankara (MVA), NYVAC (derived from the Copenhagen strain of vaccinia), and avipox vectors: canarypox (ALVAC) and fowlpox (FPV) vectors), and vesicular stomatitis virus vectors, wherein the vector for priming and the vector for boosting are different or the same.

In a further aspect, the present invention relates to a CD81-binding peptide, which corresponds to amino acids 28 to 364 of the amino acid sequence set forth in SEQ ID NO: 3 or 9 or variants thereof, or to amino acids 28 to 363 of the amino acid sequence set forth in SEQ ID NO: 5 or 7 or variants thereof.

In a further aspect, the present invention relates to a polypeptide comprising the CD81-binding peptide of the invention, with the proviso that said polypeptide is not a wild type E2.

In another aspect, the present invention provides a polynucleotide encoding the CD81-binding peptide of the present invention or the polypeptide comprising the CD81-binding peptide of the present invention.

In a further aspect, the present invention relates to an expression cassette comprising (i) the polynucleotide of the present invention and (ii) one or more polynucleotides selected from the group consisting of poly-adenylation signal, promoter, enhancer, a nucleotide sequence encoding a heterologous protein, and a nucleotide sequence encoding a peptide-tag.

In a further aspect, the present invention relates to a vector comprising the polynucleotide of the present invention or the expression cassette of the present invention.

In a further aspect, the present invention relates to a vector comprising (i) a polynucleotide encoding a CD81-binding peptide of HCV E2, which is devoid of or mutated within the N-terminal 27 amino acids of the mature E2 envelope glycoprotein, i.e., the hypervariable region 1 (HVR1) or variant thereof which retains the ability to bind to CD81, (ii) a polynucleotide encoding a polypeptide comprising said CD81-binding peptide with the proviso that said polypeptide is not a wild type E2, or (iii) an expression cassette comprising (a) the polynucleotide of (i) or (ii) and (b) one or more polynucleotides selected from the group consisting of poly-adenylation signal, promoter, enhancer, a nucleotide sequence encoding a heterologous protein, and a nucleotide sequence encoding a peptide-tag.

In a preferred embodiment of all aspects of the present invention, the vector is selected from the group consisting of a plasmid DNA vector, a viral vector, a viral-like particle, a bacterial spore, and a bacteriophage, wherein preferably the vector is a plasmid DNA, an adenovirus (Ad) vector (e.g., a non-replicating Ad5, Ad11, Ad26, Ad35, Ad49, ChAd3, ChAd4, ChAd5, ChAd6, ChAd7, ChAd8, ChAd9, ChAd10, ChAd11, ChAd16, ChAd17, ChAd19, ChAd20, ChAd22, ChAd24, ChAd26, ChAd30, ChAd31, ChAd37, ChAd38, ChAd44, ChAd63, ChAd82, ChAd55, ChAd73, ChAd83, ChAd146, ChAd147 PanAd1, PanAd2, and PanAd3 vector or a replication-competent Ad4 and Ad7 vector), an adeno-associated virus (AAV) vector (e.g., an AAV type 5), an alphavirus vector (e.g., Venezuelan equine encephalitis virus (VEE), sindbis virus (SIN), semliki forest virus (SFV), and VEE-SIN chimeras), a herpes virus vector, a measles virus vector, a pox virus vector (e.g., vaccinia virus, modified vaccinia virus Ankara (MVA), NYVAC (derived from the Copenhagen strain of vaccinia), and an avipox vector such as a canarypox (ALVAC) and fowlpox virus (FPV) vector), and a vesicular stomatitis virus vector. In a particularly preferred embodiment of all aspects of the present invention, the vector is selected from, preferably replication-defective, ChAd55, ChAd73, ChAd83, ChAd146, ChAd147, PanAd1, PanAd2, and PanAd3 vectors.

In a further aspect, the present invention relates to a composition comprising the CD81-binding peptide of the present invention, a polypeptide comprising the CD81-binding peptide of the present invention, a polynucleotide of the present invention, an expression cassette of the present invention, or a vector of the present invention, and an adjuvant.

In a further aspect, the present invention relates to a pharmaceutical composition comprising the CD81-binding peptide of the present invention, a polypeptide comprising the CD81-binding peptide of the present invention, a polynucleotide of the present invention, an expression cassette of the present invention, a vector of the present invention, or a composition of the present invention, and a pharmaceutically acceptable excipient, carrier, or diluent.

### BRIEF DESCRIPTION OF THE DRAWINGS

FIG. 1: Neutralization of sera from mice immunized with adenovirus vector encoding for wild type envelope glycoprotein E2 (Ad6E2). Sera from individual mice were tested for their neutralizing activity against HCVcc from different genotypes (indicated in the inset at the top of the figure). Sera from individual mice are indicated on the horizontal axis. On the vertical axis is shown the neutralization level of the sera for each individual HCVcc.

FIG. 2: Neutralization of sera from mice immunized with adenovirus vector encoding for envelope glycoprotein E2 lacking HVR1 (Ad6DeltaE2). Sera from individual mice were tested for their neutralizing activity against HCVcc from different genotypes (indicated in the inset at the top of the figure). Sera from individual mice are indicated on the horizontal axis. On the vertical axis is shown the neutralization level of the sera for each individual HCVcc.

FIG. 3: Alignment of the amino acid sequences of the envelope glycoprotein E2 from HCV isolates T212, BK, H77, and N2 using the publicly available alignment software ClustalW (http://www.ebi.ac.uk/Tools/clustalw2/index.html) using the default parameters (Protein Gap Open Penalty=10.0; Protein Gap Extension Penalty=0.2; Protein matrix=Gonnet; Protein/DNA ENDGAP=−1; Protein/DNA GAPDIST=4).

### DETAILED DESCRIPTION OF THE INVENTION

### Definitions

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, H. G. W. Leuenberger, B. Nagel, and H. Kölbl, Eds., Helvetica Chimica Acta, CH-4010 Basel, Switzerland, (1995).

The practice of the present invention will employ, unless otherwise indicated, conventional methods of chemistry, biochemistry, recombinant DNA techniques, and immunological methods, within the skill of the art. Such techniques are explained fully in the literature (cf., e.g., Fundamental Virology, 3^{rd} Edition, B. N. Fields and D. M. Knipe eds., Raven Press, New York 1996; Handbook of Experimental Immunology, D. M. Weir and C. C. Blackwell eds., Blackwell Scientific Publications, Oxford 1973; Molecular Cloning: A Laboratory Manual, 2^{nd} Edition, J. Sambrook et al. eds., Cold Spring Harbor Laboratory Press, Cold Spring Harbor 1989).

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

As used in this specification and the appended claims, the singular forms “a”, “an”, and “the” include plural referents, unless the content clearly dictates otherwise. Thus, for example, reference to “an immunogen” includes a mixture of two or more immunogens, and the like.

The term “hepatitis C virus” (HCV) refers to any HCV genotype, e.g., genotype 1, 1a, 1b, 1c, 2, 2a, 2b, 2c, 3, 3a, 3b, 4, 4a, 4b, 4c, 4d, 4e, 5, 5a, 6, 6a, 7, 7a, 7b, 8, 8a, 8b, 9, 9a, 10, 10a, 11, and 11a., and strains and isolates encompassed by these genotypes, for example, as disclosed by the Viral Bioinformatics Resource Center (http://www.hcvdb.org/viruses.asp). Particularly preferred isolates are T212 (genotype 1b), BK (genotype 1b), H77 (genotype 1a), and N2 (genotype 1b). The HCV contains a 9.5 kb positive-sense RNA genome that is translated into a protein containing approximately 3000 amino acids, i.e., the HCV polyprotein, that is further cleaved by cellular and viral proteases into separate structural and nonstructural HCV proteins. Preferred genotypes are 1a, 1b, 1c, 2a, 2b, 2c, 3a, and 3b. Particularly preferred genotypes are 1a and 1b.

The term “E2 protein” designates a polypeptide derived from an HCV E2 region and refers to the envelope glycoprotein 2 of HCV, which preferably corresponds to amino acids 384 to 746 or 747 of the HCV polyprotein, wherein the exact amino acid position within the polyprotein may be shifted depending on the HCV genotype. The E2 protein can be derived from any HCV genotype and strains and isolates as defined above. Preferably the E2 protein corresponds to or consists of an amino acid sequence as set forth in SEQ ID NO: 3, 5, 7, or 9, and most preferably corresponds to or consists of an amino acid sequence as set forth in SEQ ID NO: 9. Furthermore, the E2 protein may comprise a signal sequence, e.g., a signal peptide that begins at approximately amino acid 370 of the HCV polyprotein (preferably SEQ ID NO: 11, 12, or 13) corresponding to the about 14 carboxy terminal amino acids of the envelope glycoprotein E1 or a heterologous signal sequence, such as the signal peptide of the tissue plasminogen activator (tPA) as set forth in SEQ ID NO: 15 (nucleotide sequence set forth in SEQ ID NO: 14).

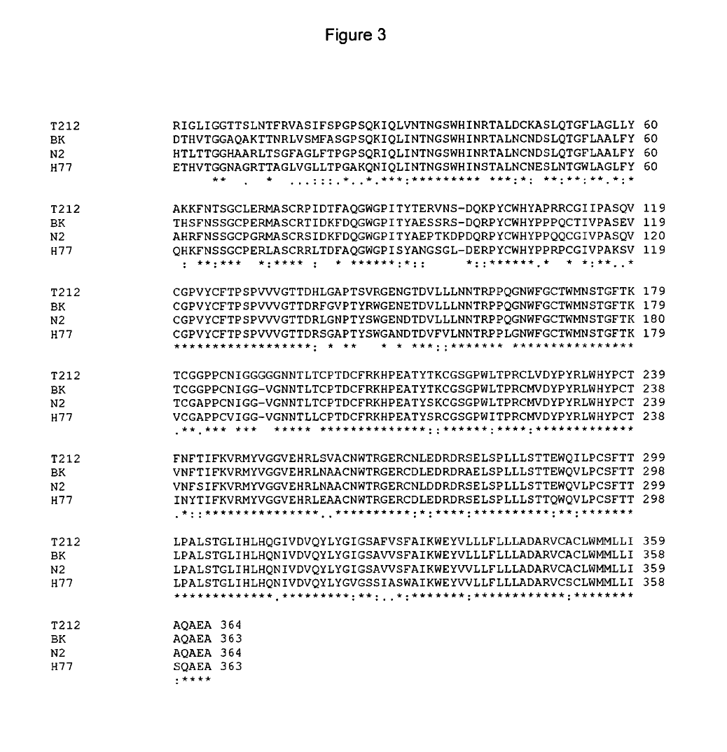

The E2 protein includes an amino terminal “hypervariable region 1 (HVR1)”, which corresponds to amino acids 384 to 410 of the HCV polyprotein. Preferably, said HVR1 has an amino acid sequence of amino acid positions 1 to 27 of SEQ ID NO: 3, 5, 7, or 9. Moreover, the E2 protein contains a carboxy terminal transmembrane domain (TMD) which starts at approximately amino acid positions 715-730 of the HCV polypeptide and may extend as far as amino acid residue 747. For example, the E2 TMDs of the HCV isolates T212 (SEQ ID NO: 3) and N2 (SEQ ID NO: 9) extend from amino acid position 718 to 747 of the HCV polypeptide, the E2 TMD of the HCV isolates BK (SEQ ID NO: 5) and H77 (SEQ ID NO: 7) extend from amino acid position 717 to 746 of the HCV polypeptide. The E2 protein as defined herein may or may not include the TMD or parts thereof. For example, the E2 protein may be carboxy terminally truncated such that the carboxy terminal amino acid residue corresponds to position 746, 745, 717, 716, 715, 714, 685, 684, 683, 682, 666, 665, 664, 663, 662, 661, or 660 of the HCV polyprotein of the HCV isolates T212 and N2, i.e., positions 363, 362, 334, 333, 332, 331, 302, 301, 300, 299, 283, 282, 281, 280, 279, 278, or 277 of the amino acid sequences set forth in SEQ ID NO: 3 or 9, or to positions 745, 744, 716, 715, 714, 713, 684, 683, 682, 681, 665, 664, 663, 662, 661, 660, or 659 of the HCV polyprotein of the HCV isolates BK and H77, i.e., positions 362, 361, 333, 332, 331, 330, 301, 300, 299, 298, 282, 281, 280, 279, 278, 277, or 276 of the amino acid sequences set forth in SEQ ID NO: 5 or 7. The skilled person is well aware of how to determine the corresponding amino acid positions in E2 proteins derived from other genotypes, strains, or isolates. For example, a given E2 amino acid sequence may be aligned with any or all of the amino acid sequences set forth in SEQ ID NO: 3 (T212), 5 (BK), 7 (H77) or 9 (N2) using a standard alignment software such as ClustalW (http://www.ebi.ac.uk/Tools/clustalw2/index.html) with default settings (Protein Gap Open Penalty=10.0; Protein Gap Extension Penalty=0.2; Protein matrix=Gonnet; Protein/DNA ENDGAP=−1; Protein/DNA GAPDIST=4). Amino acid positions corresponding to the above defined carboxy terminal amino acids can be derived from such an alignment by the skilled person. Such an alignment is shown in FIG. 3.

The term “CD81-binding peptide of HCV E2” refers to peptides derived from the envelope glycoprotein E2 of HCV, the E2 protein, which have the ability to bind CD81, wherein the genotype of HCV can be any genotype as specified above. The genotype is preferably selected from the group consisting of 1a, 1b, 1c, 2, 2a, 2b, 3, 3a, 3b, 4, 5, and 6, preferably corresponding to or having an amino acid sequence as set forth in SEQ ID NO: 3, 5, 7, or 9.

A “variant of a CD81-binding peptide of HCV E2” has at least 60%, 65%, 70%, 80%, 81%, 82%, 83%, 84%, 85%, 86%, 87%, 88%, 89%, 90%, 91%, 92%, 93%, 94%, 95%, 96%, 97%, 98%, 99% sequence similarity, preferably sequence identity, preferably over the entire length of the variant using the best sequence alignment and/or over the region of the best sequence alignment, wherein the best sequence alignment is obtainable with art known tools, e.g., Align, using standard settings, preferably EMBOSS::needle, Matrix: Blosum62, Gap Open 10.0, Gap Extend 0.5, with one of the amino acid sequences set forth in SEQ ID NO: 3, 5, 7, or 9 and shows CD81 binding as defined below. The term “CD81-binding peptides of HCV E2 variants” further refers to HCV E2 derived CD81-binding peptides containing amino acid substitutions at 1, 2, 3, 4, 5, 6, 7, 8, 9, 10, 11, 12, 13, 14, 15 or more amino acid positions, and having at least 60%, 65%, 70%, 80%, 81%, 82%, 83%, 84%, 85%, 86%, 87%, 88%, 89%, 90%, 91%, 92%, 93%, 94%, 95%, 96%, 97%, 98%, 99% sequence similarity, preferably sequence identity over the entire length of the variant using the best sequence alignment and/or over the region of the best sequence alignment, wherein the best sequence alignment is obtainable with art known tools, e.g., Align, using standard settings, preferably EMBOSS::needle, Matrix: Blosum62, Gap Open 10.0, Gap Extend 0.5, with one of the amino acid sequences set forth in SEQ ID NO: 3, 5, 7, or 9. In a preferred embodiment the above indicated alignment score is obtained when aligning the sequence of the variant with SEQ ID NO: 3, 5, 7, or 9 at least over a length of 100, 110, 120, 130, 140, 150, 160, 165, 170, 180, 190, 200, 210, 220, 230, 240, 250, 260, or 270 amino acids. Thus, preferably HCV E2 variants thereof have at least 60%, 65%, 70%, 80%, 81%, 82%, 83%, 84%, 85%, 86%, 87%, 88%, 89%, 90%, 91%, 92%, 93%, 94%, 95%, 96%, 97%, 98%, 99% sequence similarity, preferably sequence identity over at least a length of 100 amino acids, when aligning the sequence of the variant with SEQ ID NO: 3, have at least 60%, 65%, 70%, 80%, 81%, 82%, 83%, 84%, 85%, 86%, 87%, 88%, 89%, 90%, 91%, 92%, 93%, 94%, 95%, 96%, 97%, 98%, 99% sequence similarity, preferably sequence identity over at least a length of 100 amino acids, when aligning the sequence of the variant with SEQ ID NO: 5, have at least 60%, 65%, 70%, 80%, 81%, 82%, 83%, 84%, 85%, 86%, 87%, 88%, 89%, 90%, 91%, 92%, 93%, 94%, 95%, 96%, 97%, 98%, 99% sequence similarity, preferably sequence identity over at least a length of 100 amino acids, when aligning the sequence of the variant with SEQ ID NO: 7, have at least 60%, 65%, 70%, 80%, 81%, 82%, 83%, 84%, 85%, 86%, 87%, 88%, 89%, 90%, 91%, 92%, 93%, 94%, 95%, 96%, 97%, 98%, 99% sequence similarity, preferably sequence identity over at least a length of 100 amino acids, when aligning the sequence of the variant with SEQ ID NO: 9, have at least 60%, 65%, 70%, 80%, 81%, 82%, 83%, 84%, 85%, 86%, 87%, 88%, 89%, 90%, 91%, 92%, 93%, 94%, 95%, 96%, 97%, 98%, 99% sequence similarity, preferably sequence identity over at least a length of 110 amino acids, when aligning the sequence of the variant with SEQ ID NO: 3, have at least 60%, 65%, 70%, 80%, 81%, 82%, 83%, 84%, 85%, 86%, 87%, 88%, 89%, 90%, 91%, 92%, 93%, 94%, 95%, 96%, 97%, 98%, 99% sequence similarity, preferably sequence identity over at least a length of 110 amino acids, when aligning the sequence of the variant with SEQ ID NO: 5, have at least 60%, 65%, 70%, 80%, 81%, 82%, 83%, 84%, 85%, 86%, 87%, 88%, 89%, 90%, 91%, 92%, 93%, 94%, 95%, 96%, 97%, 98%, 99% sequence similarity, preferably sequence identity over at least a length of 110 amino acids, when aligning the sequence of the variant with SEQ ID NO: 7, have at least 60%, 65%, 70%, 80%, 81%, 82%, 83%, 84%, 85%, 86%, 87%, 88%, 89%, 90%, 91%, 92%, 93%, 94%, 95%, 96%, 97%, 98%, 99% sequence similarity, preferably sequence identity over at least a length of 110 amino acids, when aligning the sequence of the variant with SEQ ID NO: 9, have at least 60%, 65%, 70%, 80%, 81%, 82%, 83%, 84%, 85%, 86%, 87%, 88%, 89%, 90%, 91%, 92%, 93%, 94%, 95%, 96%, 97%, 98%, 99% sequence similarity, preferably sequence identity over at least a length of 120 amino acids, when aligning the sequence of the variant with SEQ ID NO: 3, have at least 60%, 65%, 70%, 80%, 81%, 82%, 83%, 84%, 85%, 86%, 87%, 88%, 89%, 90%, 91%, 92%, 93%, 94%, 95%, 96%, 97%, 98%, 99% sequence similarity, preferably sequence identity over at least a length of 120 amino acids, when aligning the sequence of the variant with SEQ ID NO: 5, have at least 60%, 65%, 70%, 80%, 81%, 82%, 83%, 84%, 85%, 86%, 87%, 88%, 89%, 90%, 91%, 92%, 93%, 94%, 95%, 96%, 97%, 98%, 99% sequence similarity, preferably sequence identity over at least a length of 120 amino acids, when aligning the sequence of the variant with SEQ ID NO: 7, have at least 60%, 65%, 70%, 80%, 81%, 82%, 83%, 84%, 85%, 86%, 87%, 88%, 89%, 90%, 91%, 92%, 93%, 94%, 95%, 96%, 97%, 98%, 99% sequence similarity, preferably sequence identity over at least a length of 120 amino acids, when aligning the sequence of the variant with SEQ ID NO: 9, have at least 60%, 65%, 70%, 80%, 81%, 82%, 83%, 84%, 85%, 86%, 87%, 88%, 89%, 90%, 91%, 92%, 93%, 94%, 95%, 96%, 97%, 98%, 99% sequence similarity, preferably sequence identity over at least a length of 130 amino acids, when aligning the sequence of the variant with SEQ ID NO: 3, have at least 60%, 65%, 70%, 80%, 81%, 82%, 83%, 84%, 85%, 86%, 87%, 88%, 89%, 90%, 91%, 92%, 93%, 94%, 95%, 96%, 97%, 98%, 99% sequence similarity, preferably sequence identity over at least a length of 130 amino acids, when aligning the sequence of the variant with SEQ ID NO: 5, have at least 60%, 65%, 70%, 80%, 81%, 82%, 83%, 84%, 85%, 86%, 87%, 88%, 89%, 90%, 91%, 92%, 93%, 94%, 95%, 96%, 97%, 98%, 99% sequence similarity, preferably sequence identity over at least a length of 130 amino acids, when aligning the sequence of the variant with SEQ ID NO: 7, have at least 60%, 65%, 70%, 80%, 81%, 82%, 83%, 84%, 85%, 86%, 87%, 88%, 89%, 90%, 91%, 92%, 93%, 94%, 95%, 96%, 97%, 98%, 99% sequence similarity, preferably sequence identity over at least a length of 130 amino acids, when aligning the sequence of the variant with SEQ ID NO: 9, have at least 60%, 65%, 70%, 80%, 81%, 82%, 83%, 84%, 85%, 86%, 87%, 88%, 89%, 90%, 91%, 92%, 93%, 94%, 95%, 96%, 97%, 98%, 99% sequence similarity, preferably sequence identity over at least a length of 140 amino acids, when aligning the sequence of the variant with SEQ ID NO: 3, have at least 60%, 65%, 70%, 80%, 81%, 82%, 83%, 84%, 85%, 86%, 87%, 88%, 89%, 90%, 91%, 92%, 93%, 94%, 95%, 96%, 97%, 98%, 99% sequence similarity, preferably sequence identity over at least a length of 140 amino acids, when aligning the sequence of the variant with SEQ ID NO: 5, have at least 60%, 65%, 70%, 80%, 81%, 82%, 83%, 84%, 85%, 86%, 87%, 88%, 89%, 90%, 91%, 92%, 93%, 94%, 95%, 96%, 97%, 98%, 99% sequence similarity, preferably sequence identity over at least a length of 140 amino acids, when aligning the sequence of the variant with SEQ ID NO: 7, have at least 60%, 65%, 70%, 80%, 81%, 82%, 83%, 84%, 85%, 86%, 87%, 88%, 89%, 90%, 91%, 92%, 93%, 94%, 95%, 96%, 97%, 98%, 99% sequence similarity, preferably sequence identity over at least a length of 140 amino acids, when aligning the sequence of the variant with SEQ ID NO: 9, have at least 60%, 65%, 70%, 80%, 81%, 82%, 83%, 84%, 85%, 86%, 87%, 88%, 89%, 90%, 91%, 92%, 93%, 94%, 95%, 96%, 97%, 98%, 99% sequence similarity, preferably sequence identity over at least a length of 150 amino acids, when aligning the sequence of the variant with SEQ ID NO: 3, have at least 60%, 65%, 70%, 80%, 81%, 82%, 83%, 84%, 85%, 86%, 87%, 88%, 89%, 90%, 91%, 92%, 93%, 94%, 95%, 96%, 97%, 98%, 99% sequence similarity, preferably sequence identity over at least a length of 150 amino acids, when aligning the sequence of the variant with SEQ ID NO: 5, have at least 60%, 65%, 70%, 80%, 81%, 82%, 83%, 84%, 85%, 86%, 87%, 88%, 89%, 90%, 91%, 92%, 93%, 94%, 95%, 96%, 97%, 98%, 99% sequence similarity, preferably sequence identity over at least a length of 150 amino acids, when aligning the sequence of the variant with SEQ ID NO: 7, have at least 60%, 65%, 70%, 80%, 81%, 82%, 83%, 84%, 85%, 86%, 87%, 88%, 89%, 90%, 91%, 92%, 93%, 94%, 95%, 96%, 97%, 98%, 99% sequence similarity, preferably sequence identity over at least a length of 150 amino acids, when aligning the sequence of the variant with SEQ ID NO: 9, have at least 60%, 65%, 70%, 80%, 81%, 82%, 83%, 84%, 85%, 86%, 87%, 88%, 89%, 90%, 91%, 92%, 93%, 94%, 95%, 96%, 97%, 98%, 99% sequence similarity, preferably sequence identity over at least a length of 160 amino acids, when aligning the sequence of the variant with SEQ ID NO: 3, have at least 60%, 65%, 70%, 80%, 81%, 82%, 83%, 84%, 85%, 86%, 87%, 88%, 89%, 90%, 91%, 92%, 93%, 94%, 95%, 96%, 97%, 98%, 99% sequence similarity, preferably sequence identity over at least a length of 160 amino acids, when aligning the sequence of the variant with SEQ ID NO: 5, have at least 60%, 65%, 70%, 80%, 81%, 82%, 83%, 84%, 85%, 86%, 87%, 88%, 89%, 90%, 91%, 92%, 93%, 94%, 95%, 96%, 97%, 98%, 99% sequence similarity, preferably sequence identity over at least a length of 160 amino acids, when aligning the sequence of the variant with SEQ ID NO: 7, have at least 60%, 65%, 70%, 80%, 81%, 82%, 83%, 84%, 85%, 86%, 87%, 88%, 89%, 90%, 91%, 92%, 93%, 94%, 95%, 96%, 97%, 98%, 99% sequence similarity, preferably sequence identity over at least a length of 160 amino acids, when aligning the sequence of the variant with SEQ ID NO: 9. It is understood that variants of the CD81-binding peptides of HCV E2 may comprise additional amino acids not derived from E2, like, e.g., tags, enzymes etc., such additional amino acids will not be considered in such an alignment, i.e., are excluded from the calculation of the alignment score.

The term “CD81” refers to a member of the transmembrane 4 superfamily, also known as the tetraspanning family. Most of these members are cell-surface proteins that are characterized by the presence of four hydrophobic domains. The proteins mediate signal transduction events that play a role in the regulation of cell development, activation, growth and motility. CD81 is a cell surface glycoprotein that is known to complex with integrins and to be involved in mediating HCV entry. To assess binding of a given E2 protein to CD81 either the entire CD81 protein or extracellular parts thereof are used in protein-protein binding assays known in the art and described herein below. A peptide derived of E2 of HCV by mutations is considered to be “CD81-binding”, if it shows at least 20%, preferably 30%, 40%, 50%, 60%, 70%, 80%, 90%, 95%, 100% or more of the binding of an otherwise identical E2 peptide lacking the respective wild type HVR1, preferably an otherwise identical HCV E2 peptide having SEQ ID NO: 3, 5, 7, or 9 and lacking the HVR1 region. Thus, it is particularly preferred that the “CD81-binding peptide of HCV E2 mutated within the N-terminal 27 amino acids of the mature E2 protein” shows at least 20% of the ability of an otherwise identical HCV E2 peptide devoid of the HVR1, preferably an otherwise identical HCV E2 peptide having an amino acid sequence according to SEQ ID NO: 3, 5, 7, or 9 and devoid of HVR1 to bind to CD81, shows at least 30% of the ability of an otherwise identical HCV E2 peptide devoid of the HVR1, preferably an otherwise identical HCV E2 peptide having an amino acid sequence according to SEQ ID NO: 3, 5, 7, or 9 and devoid of HVR1 to bind to CD81; shows at least 40% of the ability of an otherwise identical HCV E2 peptide devoid of the HVR1, preferably an otherwise identical HCV E2 peptide having an amino acid sequence according to SEQ ID NO: 3, 5, 7, or 9 and devoid of HVR1 to bind to CD81; shows at least 50% of the ability of an otherwise identical HCV E2 peptide devoid of the HVR1, preferably an otherwise identical HCV E2 peptide having an amino acid sequence according to SEQ ID NO: 3, 5, 7, or 9 and devoid of HVR1 to bind to CD81; shows at least 60% of the ability of an otherwise identical HCV E2 peptide devoid of the HVR1, preferably an otherwise identical HCV E2 peptide having an amino acid sequence according to SEQ ID NO: 3, 5, 7, or 9 and devoid of HVR1 to bind to CD81; shows at least 70% of the ability of an otherwise identical HCV E2 peptide devoid of the HVR1, preferably an otherwise identical HCV E2 peptide having an amino acid sequence according to SEQ ID NO: 3, 5, 7, or 9 and devoid of HVR1 to bind to CD81; shows at least 80% of the ability of an otherwise identical HCV E2 peptide devoid of the HVR1, preferably an otherwise identical HCV E2 peptide having an amino acid sequence according to SEQ ID NO: 3, 5, 7, or 9 and devoid of HVR1 to bind to CD81; shows at least 90% of the ability of an otherwise identical HCV E2 peptide devoid of the HVR1, preferably an otherwise identical HCV E2 peptide having an amino acid sequence according to SEQ ID NO: 3, 5, 7, or 9 and devoid of HVR1 to bind to CD81; shows at least 95% of the ability of an otherwise identical HCV E2 peptide devoid of the HVR1, preferably an otherwise identical HCV E2 peptide having an amino acid sequence according to SEQ ID NO: 3, 5, 7, or 9 and devoid of HVR1 to bind to CD81; shows 100% or more of the ability of an otherwise identical HCV E2 peptide devoid of the HVR1, preferably an otherwise identical HCV E2 peptide having an amino acid sequence according to SEQ ID NO: 3, 5, 7, or 9 and devoid of HVR1 to bind to CD81.

A CD81-binding peptide of HCV E2, “which is devoid of HVR1” means a CD81-binding peptide derived from the E2 protein that is lacking the hypervariable region 1 (HVR), i.e., that is lacking amino acids corresponding to amino acids 384 to 410 of the HCV polypeptide. In a preferred embodiment, a CD81-binding peptide of HCV E2, which is devoid of HVR1 has an amino acid sequence that corresponds to amino acids 28 to 364 of SEQ ID NO: 3 or 9, amino acids 28 to 363 of SEQ ID NO: 5 or 7, or variants thereof which retain the ability to bind to CD81, and preferably exhibit increased CD81 binding when compared to a wild type E2 or variant thereof comprising a wild type HVR1, preferably compared to an E2 protein having the identical amino acid sequence as the CD81-binding peptide with the exception of HVR1 which is wild type.

The term “peptide” refers to a part of a protein or a full length protein which is composed of a single amino acid chain. The term “protein” comprises peptides that resume a secondary and tertiary structure and additionally refers to proteins that are made up of several amino acid chains, i.e., several subunits, forming quartenary structures.

Residues in two or more peptides are said to “correspond” to each other if the residues occupy an analogous position in the polypeptide structures. As is well known in the art, analogous positions in two or more polypeptides can be determined by aligning the polypeptide sequences based on amino acid sequence or structural similarities. Such alignment tools are well known to the person skilled in the art and can be, for example, obtained on the World Wide Web, e.g., ClustalW (www.ebi.ac.uk/clustalw) or Align (http://www.ebi.ac.uk/emboss/align/index.html) using standard settings, preferably for Align EMBOSS::needle, Matrix: Blosum62, Gap Open 10.0, Gap Extend 0.5. Those skilled in the art understand that it may be necessary to introduce gaps in either sequence to produce a satisfactory alignment. The “best sequence alignment” between two polypeptides is defined as the alignment that produces the largest number of aligned identical residues. The “region of best sequence alignment” ends and, thus, determines the metes and bounds of the length of the comparison sequence for the purpose of the determination of the similarity score, if the sequence similarity, preferably identity, between two aligned sequences drops to less than 30%, preferably less than 20%, more preferably less than 10% over a length of 10, 20, or 30 amino acids.

The term “sequence similarity” means that amino acids at the same position of the best sequence alignment are identical or similar, preferably identical. “Similar amino acids” possess similar characteristics, such as polarity, solubility, hydrophilicity, hydrophobicity, charge, or size. Similar amino acids are preferably leucine, isoleucine, and valine; phenylalanine, tryptophan, and tyrosine; lysine, arginine, and histidine; glutamic acid and aspartic acid; glycine, alanine, and serine; threonine, asparagine, glutamine, and methionine. The skilled person is well aware of sequence similarity searching tools, e.g., available on the World Wide Web (e.g., www.ebi.ac.uk/Tools/similarity.html).

The term “increased CD81 binding” in the context of this invention means that the CD81-binding peptides of HCV E2 of the invention exhibit in a preferred embodiment increased binding to CD81 when compared to wild type E2 glycoproteins having a wild type HVR1, preferably compared to an E2 protein having the identical amino acid sequence as the CD81-binding peptide with the exception of HVR1 which is wild type, in a binding assay. Such binding assays are well known to the person skilled in the art and are described herein below. For example, pull-down experiments after binding of recombinant E2 to CD81 displayed on cells or ELISA assays with recombinant CD81. Alternatively, binding of E2 to the cell surface can be analyzed by using a fluorescence-activated cell sorting (FACS)-based assay.

The term “purified” in reference to a polypeptide, does not require absolute purity such as a homogenous preparation, rather it represents an indication that the polypeptide is relatively purer than in the natural environment. Generally, a purified polypeptide is substantially free of other proteins, lipids, carbohydrates, or other materials with which it is naturally associated, preferably at a functionally significant level, for example, at least 85% pure, more preferably at least 95% pure, most preferably at least 99% pure. A skilled artisan can purify a polypeptide using standard techniques for protein purification. A substantially pure polypeptide will yield a single major band on a non-reducing polyacrylamide gel.

The term “isolated polynucleotide” refers to polynucleotides that were (i) isolated from their natural environment, (ii) amplified by polymerase chain reaction, or (iii) wholly or partially synthesized, and means a single or double-stranded polymer of deoxyribonucleotide or ribonucleotide bases and includes DNA and RNA molecules, both sense and anti-sense strands. The term comprises cDNA, genomic DNA, and recombinant DNA. A polynucleotide may consist of an entire gene, or a portion thereof. In a preferred embodiment, a polynucleotide as defined herein encodes a CD81-binding peptide of HCV E2 of any HCV genotype, strain or isolate as described above, or degenerate variants thereof. By “degenerate variants” is meant nucleic acid sequences that encode the same amino acid sequence, but in which at least one codon in the nucleotide sequence is different. Degenerate variants occur due to the degeneracy of the genetic code, whereby two or more different codons can encode the same amino acid.

The term “vector” as used herein includes any vectors known to the skilled person including plasmid vectors, cosmid vectors, phage vectors such as lambda phage, viral vectors such as adenovirus (Ad) vectors (e.g., non-replicating Ad5, Ad11, Ad26, Ad35, Ad49, ChAd3, ChAd4, ChAd5, ChAd6, ChAd7, ChAd8, ChAd9, ChAd10, ChAd11, ChAd16, ChAd17, ChAd19, ChAd20, ChAd22, ChAd24, ChAd26, ChAd30, ChAd31, ChAd37, ChAd38, ChAd44, ChAd63 and ChAd82 vectors or replication-competent Ad4 and Ad7 vectors), adeno-associated virus (AAV) vectors (e.g., AAV type 5), alphavirus vectors (e.g., Venezuelan equine encephalitis virus (VEE), sindbis virus (SIN), semliki forest virus (SFV), and VEE-SIN chimeras), herpes virus vectors, measles virus vectors, pox virus vectors (e.g., vaccinia virus, modified vaccinia virus Ankara (MVA), NYVAC (derived from the Copenhagen strain of vaccinia), and avipox vectors: canarypox (ALVAC) and fowlpox (FPV) vectors), and vesicular stomatitis virus vectors, viral like particles, or bacterial spores.

A chimpanzee adenovirus (also abbreviated herein as “ChAd” for common chimpanzee adenovirus and “PanAd” for bonobo chimpanzee adenovirus) provides a basis for reducing the adverse effects associated with the preexisting immunity in humans to common serotypes of human adenoviruses. Thus, viral vectors based on chimpanzee adenovirus represent an alternative to the use of human derived adenoviral vectors for the development of genetic vaccines (Farina SF, J Virol. 2001 December; 75(23):11603-13.; Fattori E, Gene Ther. 2006 July; 13(14):1088-96). The adenovirus types ChAd55, ChAd73, ChAd83, ChAd146, ChAd147, PanAd1, PanAd2, and PanAd3 are characterized by a complete absence of preexisting neutralizing antibody in humans directed against these adenovirus types. Thus, these adenoviruses provide a particularly valuable medical tool that can, e.g., be used for immunization and/or gene therapy.

In addition to the above mentioned adenoviral vectors, the adenovirus vectors ChAd55, ChAd73, ChAd83, ChAd146, and ChAd147 isolated from the Common Chimpanzee (Pan troglodytes) and PanAd1, PanAd2, and PanAd3 from bonobos (Pan paniscus) are also encompassed by the term “vector”.

For example, an adenovirus vector encompassed by the term “vector” in the context of the present application may be an adenovirus that has been deposited at ECACC (European Collection of Cell Culture, Porton Down, Salisbury, SP4 OJG, UK) and has a deposit number selected from the group consisting of 08110601 (ChAd83), 08110602 (ChAd73), 08110603 (ChAd55), 08110604 (ChAd147), and 08110605 (ChAd146). The deposits of the aforementioned adenoviral strains (Latin name: Mastadenovirus, Adenoviridae) have been made on Nov. 6, 2008 by Okairos AG, Elisabethenstr. 3, 4051 Basel, Switzerland. These deposits will be maintained under the terms of the Budapest Treaty on the International Recognition of the Deposit of Microorganisms for the Purposes of Patent Procedure. These deposits were made merely as a convenience for those of skill in the art and are not an admission that a deposit is required under 35 U.S.C. 112. All restrictions on the availability to the public of the deposited material will be irrevocably removed, except for the requirements specified in 37 C. F. R. 1. 808 (b), upon the granting of a patent. These adenoviruses and vectors based thereon are described in more detail in PCT/EP2009/000672 filed on Feb. 2, 2009, U.S. 61/172,624 filed on Apr. 24, 2009, and U.S. 61/174,852 filed on May 1, 2009, which are herewith incorporated in their entirety.

Furthermore, an adenovirus vector encompassed by the term “vector” in the context of the present application may be an adenovirus comprising the genomic nucleotide sequence as set forth in SEQ ID NO: 28 (PanAd1).

Furthermore, the term “vector” in the context of the present application encompasses viral vectors that carry one or more of the fiber, hexon, and/or penton proteins of ChAd55, ChAd73, ChAd83, ChAd146, ChAd147, PanAd1, PanAd2, and/or PanAd3 on their surface or a nucleotide sequence preferably in their genome encoding one or more of the fiber, hexon, and/or penton proteins of ChAd55, ChAd73, ChAd83, ChAd146, ChAd147, PanAd1, PanAd2, and/or PanAd3. Fiber, hexon, and penton proteins are adenovirus capsid proteins that represent the most surface exposed adenovirus epitopes. No neutralizing antibodies specific for the viruses ChAd55, ChAd73, ChAd83, ChAd146, ChAd147, PanAd1, PanAd2, and PanAd3 are comprised in human blood sera. Thus, one advantage of the aforementioned chimpanzee hexon, penton, and fiber protein sequences is that the sequences of these proteins can be used to improve other adenoviruses, which have been engineered for, e.g., medical purposes. For example, the capsid proteins or functional fragments thereof can be used to, e.g., replace/substitute one or more of the major structural capsid proteins or functional fragments thereof, respectively, of any adenovirus, to obtain improved recombinant adenoviruses with a reduced seroprevalence in humans.

The protein sequences of the fiber, hexon, and penton proteins of ChAd55, ChAd73, ChAd83, ChAd146, ChAd147, PanAd1, PanAd2, and PanAd3 are set forth in SEQ ID NO: 29 (ChAd55 fiber), SEQ ID NO: 30 (ChAd73 fiber), SEQ ID NO: 31 (ChAd83 fiber), SEQ ID NO: 32 (ChAd146 fiber), SEQ ID NO: 33 (ChAd147 fiber), SEQ ID NO: 34 (PanAd1 fiber), SEQ ID NO: 47 (PanAd2 fiber), SEQ ID NO: 50 (PanAd3 fiber), SEQ ID NO: 35 (ChAd55 hexon), SEQ ID NO: 36 (ChAd73 hexon), SEQ ID NO: 37 (ChAd83 hexon), SEQ ID NO: 38 (ChAd146 hexon), SEQ ID NO: 39 (ChAd147 hexon), SEQ ID NO: 40 (PanAd1 hexon), SEQ ID NO: 48 (PanAd2 hexon), SEQ ID NO: 51 (PanAd3 hexon), SEQ ID NO: 41 (ChAd55 penton), SEQ ID NO: 42 (ChAd73 penton), SEQ ID NO: 43 (ChAd83 penton), SEQ ID NO: 44 (ChAd146 penton), SEQ ID NO: 45 (ChAd147 penton), SEQ ID NO: 46 (PanAd1 penton), SEQ ID NO: 49 (PanAd2 penton), and SEQ ID NO: 52 (PanAd3 penton), respectively.

The terms “ChAd55”, “ChAd73”, “ChAd83”, “ChAd146”, “ChAd147”, “PanAd1”, “PanAd2”, and/or “PanAd3” in the context of the present invention also encompass all adenoviral vectors that carry one or more, preferably all of fiber, hexon, and penton proteins or nucleotide sequences encoding said proteins of one or more of the adenoviral vectors ChAd55, ChAd73, ChAd83, ChAd146, ChAd147, PanAd1, PanAd2, and/or PanAd3. For example, a vector in the context of the present invention may be any adenoviral vector, preferably replication-defective, which carries the fiber, hexon, and/or penton proteins or nucleotide sequences encoding for said proteins from ChAd55, or which carries the fiber, hexon, and/or penton proteins or nucleotide sequences encoding for said proteins from ChAd73, or which carries the fiber, hexon, and/or penton proteins or nucleotide sequences encoding for said proteins from ChAd83, or which carries the fiber, hexon, and/or penton proteins or nucleotide sequences encoding for said proteins from ChAd146, or which carries the fiber, hexon, and/or penton proteins or nucleotide sequences encoding for said proteins from ChAd147, or which carries the fiber, hexon, and/or penton proteins or nucleotide sequences encoding for said proteins from PanAd1, or which carries the fiber, hexon, and/or penton proteins or nucleotide sequences encoding for said proteins from PanAd2, or which carries the fiber, hexon, and/or penton proteins or nucleotide sequences encoding for said proteins from PanAd3, or which carries the fiber, hexon, and/or penton proteins or nucleotide sequences encoding for said proteins from ChAd55, ChAd73, ChAd83, ChAd146, ChAd147, PanAd1, PanAd2, and/or PanAd3, and preferably does not carry any fiber, hexon, and/or penton proteins or nucleotide sequences encoding said proteins from any other adenovirus than ChAd55, ChAd73, ChAd83, ChAd146, ChAd147, PanAd1, PanAd2, and/or PanAd3.

The adenoviral vectors are preferably replication defective. The term “replication-defective” adenovirus refers to an adenovirus that has been rendered to be incapable of replication because it has been engineered to comprise at least a functional deletion or a complete removal of a gene product that is essential for viral replication. For example, one or more genes selected from the group consisting of E1A, E1B, E2A, E2B, E3 and E4 gene can be deleted, rendered non-functional, and/or can be replaced by an expression cassette as outlined above. A skilled person is well aware of how to introduce these genomic alterations in the adenovirus, for example, in a deposited adenovirus strain. In this respect, methods of generating modified adenoviruses comprising a molecule for delivery into a target cell, which is a preferred modification of the deposited strains, have been described above.

In a particularly preferred embodiment of all aspects of the present invention, the vector or the recombinant vector is selected from the group consisting of replication-defective ChAd55, replication-defective ChAd73, replication-defective ChAd83, replication-defective ChAd146, replication-defective ChAd147, replication-defective PanAd1, replication-defective PanAd2, and replication-defective PanAd3 vectors as described above.

Vectors include expression as well as cloning vectors. Expression vectors comprise plasmids as well as viral vectors and generally contain a nucleotide sequence encoding a polypeptide along with regulatory elements for proper transcription and processing. The regulatory elements that may be present include those naturally associated with the nucleotide sequence encoding the polypeptide and exogenous regulatory elements not associated with the nucleotide sequence. Exogenous regulatory elements such as an exogenous promoter can be useful for expression in a particular host, such as in bacteria, yeast, plant, insect, or mammalian cells. Examples of regulatory elements useful for functional expression include a promoter, a terminator, a ribosome binding site, and a polyadenylation signal. Cloning vectors are generally used to engineer and amplify a certain desired DNA variant and may lack functional sequences needed for expression of the desired DNA variants. Furthermore, the vectors described herein are also used for the delivery of DNA vaccines.

Reference to “recombinant” nucleic acid or vector indicates the presence of two or more nucleic acid regions not naturally associated with each other.

The term “expression cassette” refers to a nucleic acid molecule which comprises at least one nucleic acid sequence that is to be expressed, along with its transcription and translation control sequences. Changing the expression cassette will cause the vector in which it is incorporated to direct the expression of a different sequence or combination of sequences. Because of the restriction sites being engineered to be present at the 5′ and 3′ ends, the cassette can be easily inserted, removed, or replaced with another cassette.

“Recombinant host cell”, as used herein, refers to a host cell that comprises a polynucleotide that codes for a peptide of interest, e.g., the CD81-binding peptide of HCV E2 or variant thereof according to the invention. This polynucleotide may be found inside the host cell (i) freely dispersed as such, (ii) incorporated in a vector, or (iii) integrated into the host cell genome or mitochondrial DNA. The recombinant cell can be used for expression of a polynucleotide of interest or for amplification of the polynucleotide or the vector of the invention. The term “recombinant host cell” includes the progeny of the original cell which has been transformed, transfected, or infected with the polynucleotide or the recombinant vector of the invention. A recombinant host cell may be a bacterial cell such as an E. coli cell, a yeast cell such as Saccharomyces cerevisiae or Pichia pastoris, a plant cell, an insect cell such as SF9 or Hi5 cells, or a mammalian cell. Preferred examples of mammalian cells are Chinese hamster ovary (CHO) cells, green African monkey kidney (COS) cells, human embryonic kidney (HEK 293) cells, HELA cells, Huh7.5 human hepatoma cells, Hep G2 human hepatoma cells, Hep 3B human hepatoma cells and the like.

As used herein, “vaccine” refers to a pharmaceutical composition comprising an immunogen that serves to stimulate an immune response to an HCV antigen, wherein the immunogen is preferably a CD81-binding peptide of HCV E2, which is devoid of or mutated within HVR1. The vaccine may serve prophylactic and/or therapeutic purposes. The immune response need not provide complete protection and/or treatment against HCV. Preferably, the protective and/or therapeutic immune response is directed against homologous and heterologous HCV infection. Preferably, the protective and/or therapeutic immune response induced by the vaccine exhibits broad specificity, e.g., by the induction of cross-reactive antibodies. In some cases, a vaccine will include an immunological adjuvant in order to enhance the immune response. The vaccine or pharmaceutical composition according to the present invention may contain the immunogen, i.e., the CD81-binding peptide of HCV E2 according to the present invention, as protein, or the vaccine may contain a nucleic acid encoding the immunogen of the invention, e.g., a DNA vaccine.

By “therapeutically effective amount” is meant an amount of CD81-binding peptide of HCV E2 according to the present invention, a polynucleotide encoding the CD81-binding peptide according to the present invention, the expression cassette according to the present invention, or the vector according to the present invention which will induce an immunological response in the individual to which it is administered. Such a response will generally result in the development of a secretory, cellular and/or antibody-mediated immune response to the immunogen, i.e., the CD81-binding peptide of HCV E2, which is devoid of or mutated within the HVR1 according to the present invention. Usually, such a response includes but is not limited to one or more of the following effects: the production of antibodies from any of the immunological classes, such as immunoglobulins A, D, E, G, or M; the proliferation of B and T lymphocytes; the provision of activation, growth, and differentiation signals to immunological cells; expansion of helper T cell, suppressor T cell, and/or cytotoxic T cell and/or γδ T cell populations.

The term “adjuvant” as used herein refers to substances, which when administered prior, together or after administration of an antigen/immunogen accelerates, prolong and/or enhances the quality and/or strength of an immune response to the antigen in comparison to the administration of the antigen alone, thus, reducing the quantity of antigen/immunogen necessary in any given vaccine, and/or the frequency of injection necessary in order to generate an adequate immune response to the antigen/immunogen of interest.

The term “antibody” refers to both monoclonal and polyclonal antibodies, i.e., any immunoglobulin protein or portion thereof which is capable of recognizing an antigen or hapten within the CD81-binding peptide of HCV E2, or variant thereof. Antigen-binding portions may be produced by recombinant DNA techniques or by enzymatic or chemical cleavage of intact antibodies. In some embodiments, antigen-binding portions include Fab, Fab′, F(ab′)₂, Fd, Fv, dAb, and complementarity determining region (CDR) variants, single-chain antibodies (scFv), chimeric antibodies such as humanized antibodies, diabodies, and polypeptides that contain at least a portion of an antibody that is sufficient to confer specific antigen binding to the polypeptide.

The term “induction of cross-reactive antibodies” means capable of inducing antibodies that are effective in the prevention or treatment of infection by heterologous HCV strains.

By “infection by heterologous HCV strains” is meant an infection by an HCV strain whose sequence in the envelope glycoprotein E2 is different from that from which the used immunogen is derived. In contrast, by “infection by a homologous HCV strain” is meant an infection by an HCV strain with a genotype or closely related to the genotype from which the envelope protein E2 used as immunogen is derived.

The administration of an immunogen for inducing/generating an immune response against HCV in a mammal in the context of the present invention is termed “priming”, and the administration of an immunogen for enhancing an immune response against HCV in a mammal is termed “boosting”. Priming and boosting may be performed using the antigen/immunogen as protein. In a preferred embodiment priming and boosting may be performed using a vector containing a nucleic acid sequence encoding the CD81 binding peptide of the present invention, wherein the vector for priming and the vector for boosting may be the same or different. The term “heterologous prime-boost” means that the vector for inducing/generating an immune response (priming) against HCV in a mammal and the vector for enhancing the immune response (boosting) against HCV in a mammal are different. In a preferred embodiment of heterologous prime-boost two different adenovirus vectors are used that are not cross-reacting.

A “patient” refers to a mammal capable of being infected with HCV. Examples of patients are humans and chimpanzees.

The term “excipient” when used herein is intended to indicate all substances in a pharmaceutical formulation which are not active ingredients such as, e.g., carriers, binders, lubricants, thickeners, surface active agents, preservatives, emulsifiers, buffers, flavoring agents, or colorants.

The term “pharmaceutically acceptable carrier” includes, for example, magnesium carbonate, magnesium stearate, talc, sugar, lactose, pectin, dextrin, starch, gelatin, tragacanth, methylcellulose, sodium carboxymethylcellulose, a low melting wax, cocoa butter, and the like.

### DESCRIPTION

The present inventors have found that CD81-binding peptides derived from the HCV envelope protein E2, which are devoid of or mutated within the hypervariable region 1 (HVR1) are capable of inducing a broad-specificity immune response when used as immunogen in a pharmaceutical composition (vaccine).

Without being bound to any theory, it is believed that mutation or deletion of HVR1 leads to increased binding to CD81, which indicates enhanced exposure of CD81 binding sites within E2 compared to wild type E2. When used as antigen/immunogen, for example, in a pharmaceutical composition or vaccine, these exposed CD81-binding sites may serve as epitopes for the generation of an immune response, preferably the generation of antibodies directed against said CD81 binding sites. Such antibodies may interfere with the interaction between CD81, an HCV receptor, and the HCV envelope proteins, and thus, interfere with or inhibit an HCV infection by inhibiting the docking and entry of the virus.

In one aspect, the present invention relates to a CD81-binding peptide of hepatitis C virus (HCV) envelope protein E2, which is devoid of the hypervariable region 1 (HVR1), i.e., which lacks the 27 amino terminal amino acids, or which is mutated within the HVR1. In a preferred embodiment of the CD81-binding peptide of HCV E2 according to the present invention, N amino acids of the HVR1 are mutated or deleted, wherein N is any number between 1 and 27, i.e., 1, 2, 3, 4, 5, 6, 7, 8, 9, 10, 11, 12, 13, 14, 15, 16, 17, 18, 19, 20, 21, 22, 23, 24, 25, 26, or 27.

In an even more preferred embodiment, said CD81-binding peptide that is devoid of or mutated within the HVR1 exhibits increased binding to CD81 when compared to wild type E2 glycoproteins having a wild type HVR1, preferably compared to an E2 protein having the identical amino acid sequence as the CD81-binding peptide with the exception of HVR1 which is wild type.

The skilled person is well aware of experiments for testing binding to CD81. For example, cells expressing CD81, e.g., CHO cells transfected with an expression construct encoding CD81 or MOLT-4 cells, are incubated with the CD81-binding peptide of the present invention or variants thereof, e.g., as culture supernatant of cells expressing and secreting recombinant soluble CD81-binding peptides of the invention, preferably concentrated, or in the form of a cell extract of recombinant cells expressing the CD81-binding peptides of the present invention. For the purpose of testing for CD81-binding it is preferred that the CD81-binding peptides are fused to a peptide or protein tag such as a His-tag. After washing and lysis of the cells, the lysates are analyzed by Western blotting for the detection of cell-bound CD81-binding peptide. For example, for detection of recombinant E2 protein of genotype 1a, the rat monoclonal antibody 6-1/a (Flint et al., 1999) may be used in combination with a secondary antibody comprising a detectable marker or an enzyme capable of generating a detectable signal such as horseradish peroxidase. Alternatively, a primary antibody directed against the peptide or protein tag such as an anti-His-tag antibody, which are commercially available, may be used in combination with an appropriate secondary antibody.

Alternatively, pull down experiments using GST-fusion proteins comprising a CD81 protein, e.g., the CD81 large extracellular loop (LEL), may be incubated with culture supernatant of cells expressing and secreting recombinant soluble CD81-binding peptides of the invention, preferably concentrated, or with a cell extract of recombinant cells expressing the CD81-binding peptides of the present invention. In a preferred embodiment, the CD81-binding peptides are fused to a peptide or protein tag such as a His-tag. The mixture is then incubated, e.g., with glutathione beads. After washing and elution of the beads the CD81-binding peptides bound to the GST-CD81 fusion protein, e.g., the GST-CD81 LEL fusion protein, may be analyzed by Western blotting as described above using antibodies directed against the CD81-binding peptide or the peptide or protein tag, for example, the His-tag. Instead, the GST-CD81 protein may be immobilized on an enzyme-linked immunosorbent assay (ELISA) plate which is then incubated with culture supernatant of cells expressing and secreting recombinant soluble CD81-binding peptides of the invention, preferably concentrated, or with a cell extract of recombinant cells expressing the CD81 binding peptides of the present invention. After washing the plate, the bound CD81-binding peptides are detected using antibodies directed against the CD81-binding peptide or the peptide or protein tag such as anti-His-tag antibodies, which are well known in the field, and incubation with a secondary antibody comprising a detectable marker or an enzyme that is capable of generating a detectable signal such as alkaline phosphatase. If applicable, the plate is then incubated with the appropriate substrate, i.e., in the case of alkaline phosphatase for example with p-nitrophenyl phosphate, and the signal is detected using an ELISA reader. The intensity of the signal indicates the extent of binding.

Alternatively, binding of the CD81-binding peptides of the invention to CD81 may be analyzed by a fluorescence-activated cell sorting (FACS)-based assay. Cells expressing CD81, e.g., CHO cells transfected with an expression construct encoding CD81 or MOLT-4 cells, are incubated with the CD81-binding peptide of the present invention or variants thereof, preferably fused to a protein or peptide tag such as a His-tag, e.g., as culture supernatant of cells expressing and secreting recombinant soluble CD81-binding peptides, preferably concentrated, or in the form of a cell extract of recombinant cells expressing the CD81-binding peptides of the present invention. The cells are washed and bound CD81-binding peptides are detected using antibodies directed against the CD81-binding peptide or the peptide or protein tag such as anti-His-tag antibodies in combination with a fluorescently labeled secondary antibody, e.g., a phycoerythrin conjugated antibody. Flow cytometry data acquisition is performed on a FACS machine such as FACSCalibur (Becton Dickinson) and analyzed with appropriate analysis software such as the CellQuest software (Becton Dickinson).

In one embodiment of this aspect of the invention, the HVR1 is mutated in “N” amino acids of the 27 amino terminal amino acids, for example, in the amino acids corresponding to amino acids 1 to 27 as set forth in amino acid sequence SEQ ID NO: 3, 5, 7, or 9, wherein “N” is selected from 1, 2, 3, 4, 5, 6, 7, 8, 9, 10, 11, 12, 13, 14, 15, 16, 17, 18, 19, 20, 21, 22, 23, 24, 25, 26, and 27 amino acids. For example, CD81-binding peptides of HCV E2 having the following mutations within the HVR1 at 1 to 27 amino acid positions, i.e., at 1, 2, 3, 4, 5, 6, 7, 8, 9, 10, 11, 12, 13, 14, 15, 16, 17, 18, 19, 20, 21, 22, 23, 24, 25, 26, or 27 amino acid positions, compared to amino acids 1 to 27 of the amino acid sequence set forth in SEQ ID NO: 3, 5, 7, or 9, and lead to increased CD81-binding in the context of a wild type CD81-binding peptide of HCV E2 are encompassed by the present invention. Preferably, the following amino acids are substituted at amino acid position 1: Cys, Phe, Leu, Met, Pro, Trp, or Tyr; amino acids Ala, Cys, Asp, Glu, Phe, Gly, His, Ile, Lys, Leu, Met, Asn, Pro, Gln, Arg, Val, Trp, or Tyr at amino acid position 2; amino acids Ala, Asp, Glu, Phe, Gly, Lys, Pro, or Trp at amino acid position 3; amino acids Asp, Glu, Phe Gly, Lys, Asn, Pro, or Trp at amino acid position 4; amino acids Cys, Asp, Glu, Gly, His, Lys, Asn, Pro, Arg, or Tyr at amino acid position 5; amino acids Cys, Asp, Glu, Phe, His, Ile, Lys, Leu, Met, Asn, Pro, Gln, Arg, Ser, Thr, Val, Trp, or Tyr at amino acid position 6; amino acids Cys, Asp, Phe, Ile, Lys, Leu, Asn, Pro, Thr, Trp, or Tyr at amino acid position 7; amino acids Asp, Phe, Gly, Met, Pro, Trp, or Tyr at amino acid position 8; amino acids Cys, Asp, Phe, Gly, His, Ile, Lys, or Trp at amino acid position 9; amino acids Cys, Asp, Phe, His, Ile, Lys, Leu, Met, Asn, Pro, Gln, Arg, Thr, Val, Trp, or Tyr at amino acid position 10; amino acids Asp, Ile, Leu, Met, Asn, Pro, Thr, Val, or Trp at amino acid position 11; amino acids Cys, Phe, Lys, Met, or Trp at amino acid position 12; amino acids Cys, Asp, Flu, Phe, Gly, His, Lys, Asn, Pro, Gln, Arg, Ser, Trp, or Tyr at amino acid position 13; amino acids Cys, Asp, Glu, Ile, or Pro at amino acid position 14; amino acids Cys, Asp, Glu, Phe, His, Leu, Met, Pro, Trp, or Lys at amino acid position 15; amino acids Ala, Cys, Asp, Glu, His, Lys, Met, Asn, Pro, Gln, Arg, Thr, or Trp at amino acid position 16; amino acids Cys, Asp, Glu, Phe, Gly, His, Ile, Lys, Leu, Met, Gln, or Trp at amino acid position 17; amino acids Cys, Glu, Phe, His, Ile, Met, Pro, Gln, Val, Trp, or Lys at amino acid position 18; amino acids Ala, Cys, Asp, Glu, Gly, His, Lys, Asn, Pro, Gln, Arg, Thr, Val, Trp, or Tyr at amino acid position 19; amino acids Ala, Cys, Asp, Glu, Gly, His, Ile Lys, Met, Asn, Pro, Gln, Arg, Thr, Val, Trp, or Tyr at amino acid position 20; amino acids Cys, Glu, Phe, His, Ile, Lys, Pro, Val, Trp, or Tyr at amino acid position 21; amino acids Cys, Asp, Glu, Gly, or Asn at amino acid position 22; amino acids Cys, Asp, Glu, Phe, His, Ile, Lys, Met, Asn, Pro, Gln, Arg, Ser, Thr, Val, Trp, or Tyr at amino acid position 23; amino acids Cys, Asp, Glu, Phe, His, Ile, Lys, Leu, Met, Asn, Thr, Val, Trp, or Tyr at amino acid position 24; amino acids Cys, Asp, Glu, Phe, Gly, Ile, Leu, or Pro at amino acid position 25; amino acids Ala, Cys, Asp, Phe, Gly, Ile, Lys, Leu, Met, Asn, Pro, Ser, Thr, Val, Trp, or Tyr at amino acid position 26, and/or amino acids Ala, Cys, Phe, Gly, Ile, Met, Pro, or Trp at amino acid position 27. In a preferred embodiment, positions 2, 6, 7, 10, 13, 16, 19, 20, 23, and/or 26 or any combination thereof are substituted with the above indicated amino acids for the corresponding amino acid positions. In a more preferred embodiment positions 2, 6, 7, 16, 19, 20, 23, and/or 26 or any combination thereof are substituted with the above indicated amino acids for the corresponding amino acid positions.

Any CD81-binding peptide of E2 of any HCV genotype as defined above comprising any of the above defined HVR1 mutations either alone or in combination is encompassed by the present invention.

In a preferred embodiment the CD81-binding peptide is derived from a naturally occurring HCV genotype selected from the group consisting of genotype 1, 1a, 1b, 1c, 2, 2a, 2b, 2c, 3, 3a, 3b, 4, 4a, 4b, 4c, 4d, 4e, 5, 5a, 6, 6a, 7, 7a, 7b, 8, 8a, 8b, 9, 9a, 10, 10a, 11 and 11a. Particularly preferred are genotypes 1a, 1b, 1c, 2, 2a, 2b, 3, 3a, 3b, 4, 5, and 6. Even more preferred genotypes are 1a and 1b.

Furthermore, all HCV strains/isolates are encompassed by the present invention. The sequences of preferred genotypes, strains, and isolates encompassed by the present invention are, for example, obtainable on the Homepage of the Viral Bioinformatics Resource Center (http://www.hcvdb/viruses.asp). Particularly preferred HCV isolates for all aspects of the present invention are HCV genotype 1b isolate T212, HCV genotype 1b isolate BK, HCV genotype 1a isolate H77, and HCV genotype 1b isolate N2. In a preferred embodiment, the CD81-binding peptide according to the present invention corresponds to or consists of amino acids 28 to 364 of the amino acid sequences set forth in SEQ ID NO: 3 or 9, amino acids 28 to 363 of the amino acid sequences set forth in SEQ ID NO: 5 or 7, or variants thereof, which preferably retain the ability to bind to CD81, more preferably exhibit increased CD81 binding when compared to the accordant CD81-binding peptides or variants thereof comprising a wild type HVR1, preferably compared to an E2 protein having the identical amino acid sequence as the CD81-binding peptide with the exception of HVR1 which is wild type.

In another preferred embodiment, the CD81-binding peptide of the invention corresponds to or consists of amino acids 28 to 363, 28 to 362, 28 to 334, 28 to 333, 28 to 332, 28 to 331, 28 to 302, 28 to 301, 28 to 300, 28 to 299, 28 to 283, 28 to 282, 28 to 281, 28 to 280, 28 to 279, 28 to 278, or 28 to 277 as set forth in SEQ ID NO: 3 or 9, or amino acids 28 to 362, 28 to 361, 28 to 333, 28 to 332, 28 to 331, 28 to 330, 28 to 301, 28 to 300, 28 to 299, 28 to 298, 28 to 282, 28 to 281, 28 to 280, 28 to 279, 28 to 278, 28 to 277, or 28 to 276 as set forth in SEQ ID NO: 5 or 7.

In another preferred embodiment, the CD81-binding peptide of the invention corresponds to the amino acid sequence set forth in SEQ ID NO: 3, 5, 7, or 9, wherein the first 27 amino terminal amino acids are mutated in N amino acids, wherein “N” is selected from 1, 2, 3, 4, 5, 6, 7, 8, 9, 10, 11, 12, 13, 14, 15, 16, 17, 18, 19, 20, 21, 22, 23, 24, 25, 26, and/or 27 amino acids. In this embodiment, it is preferred that the mutation(s) within the HVR1 result(s) in increased CD81 binding when compared to wild type E2. Such HVR1 mutations are described above. In another preferred embodiment, the CD81-binding peptide of the invention corresponds to amino acids 1 to 363, 1 to 362, 1 to 334, 1 to 333, 1 to 332, 1 to 331, 1 to 302, 1 to 301, 1 to 300, 1 to 299, 1 to 283, 1 to 282, 1 to 281, 1 to 280, 1 to 279, 1 to 278, or 1 to 277 as set forth in SEQ ID NO: 3 or 9, or amino acids 1 to 362, 1 to 361, 1 to 333, 1 to 332, 1 to 331, 1 to 330, 1 to 301, 1 to 300, 1 to 299, 1 to 298, 1 to 282, 1 to 281, 1 to 280, 1 to 279, 1 to 278, 1 to 277, or 1 to 276 as set forth in SEQ ID NO: 5 or 7, wherein the first 27 amino terminal amino acids are mutated in N amino acids, wherein N is selected from 1, 2, 3, 4, 5, 6, 7, 8, 9, 10, 11, 12, 13, 14, 15, 16, 17, 18, 19, 20, 21, 22, 23, 24, 25, 26, 27 amino acids. In this embodiment, it is preferred that the mutation(s) within the HVR1 result(s) in increased CD81 binding when compared to wild type E2 glycoproteins having a wild type HVR1, preferably compared to an E2 protein having the identical amino acid sequence as the CD81-binding peptide with the exception of HVR1 which is wild type. Such HVR1 mutations are described above.

In another aspect, the invention encompasses a polypeptide comprising any of the above described CD81-binding peptides of HCV E2 which are devoid of or mutated within the HVR1, preferably derived from a naturally occurring HCV genotype. In a preferred embodiment, the present invention provides a polypeptide comprising any of the above defined CD81-binding peptides fused in frame to another protein or protein variant, wherein the protein or variant thereof may be either heterologous (not derived from HCV) or homologous (derived from HCV), with the proviso that said polypeptide is not wild type E2. In an even more preferred embodiment of this aspect of the invention, said polypeptide exhibits increased CD81 binding when compared to wild type E2 glycoproteins having a wild type HVR1, preferably compared to an E2 protein having the identical amino acid sequence as the CD81-binding peptide with the exception of HVR1 which is wild type.

In a preferred embodiment, the heterologous protein may be Interleukin 2 (IL2), Ubiquitin, the antibody fragment crystallizable region (Fc region), Furin cleavage sites, the T helper universal epitope from the B subunit of the heat labile enterotoxin (LTB), or glycoprotein D (gD) of herpes simplex virus (HSV). In a particular preferred embodiment, the heterologous protein is selected from the group consisting of Furin cleavage sites, the T helper universal epitope from the B subunit of the heat labile enterotoxin (LTB), and the gD protein of HSV.

Furin cleaves C-terminally to RxRR or RxKR where x is any amino acid. An example of a Furine cleavage site is REKR, a motif that occurs in HIV gp160, (Hallenberger et al., 1997). RHRR and RKRR occur in human TGFbeta and IGFR-1, respectively. One constraint on x when using RxR/KR to separate epitopes or proteins is to avoid creating neo-epitopes in the junctional region. Neo-epitopes are not necessarily dangerous by themselves, but may create competition with the target epitopes. One way to avoid most junctional epitopes is by not allowing x to be one of the aliphatic amino acids (L,M,V,I,A), as they are most often found at anchor positions of A2-restricted epitopes.

The B subunit of heat-labile enterotoxin (LTB) fused to carcinoembryonic antigen elicits antigen-specific immune responses and antitumor effects (Facciabene et al., 2007)

It has been shown that viral antigens fused to glycoprotein D (gD) of herpes simplex virus induced T and B cell responses to the antigen that were far more potent than those elicited by the same antigen expressed without gD (Lasaro et al., 2008).

Another example for a heterologous protein comprised by the polypeptide of the present invention is the tissue plasminogen activator (tPA). In a preferred embodiment, the secretion signal sequence of tPA, which preferably corresponds to or consists of the amino acid sequence set forth in SEQ ID NO: 15, preferably encoded by the nucleotide sequence set forth in SEQ ID NO: 14, is fused to the amino terminus of the CD81-binding peptide of the present invention.

In one embodiment of this aspect of the invention, the polypeptide does not comprise naturally occurring HCV sequences apart from the CD81-binding peptide of the invention, which is preferably derived from a naturally occurring HCV genotype. In another embodiment, the polypeptide does not comprise naturally occurring HCV E1 or E2 sequences apart from the CD81-binding peptide of the invention.

In another embodiment of this aspect of the invention, the homologous protein comprised by the polypeptide of the invention may be any of the HCV proteins apart from E2, i.e., capsid, E1, NS2, NS3, NS4A, NS4B, NS5A, or NS5B. In a preferred embodiment, the homologous protein comprised by the polypeptide of the invention is the envelope glycoprotein E1. The amino acid sequence may be derived from any of the above described HCV genotypes, strains, or isolates. In a preferred embodiment, the E1 protein is derived from the HCV isolates T212, BK, H77, or N2. In a more preferred embodiment, the amino acid sequence of the E1 protein corresponds to or consists of one of the amino acid sequences set forth in SEQ ID NO: 17, 19, 21, and 23. The E1 protein comprised by the polypeptide of this embodiment of the invention may be the full-length E1 protein or fragments thereof. In a preferred embodiment the E1 protein fragment corresponds to or consists of the 14 carboxy-terminal amino acids of the E1 protein. In a preferred embodiment, the E1 protein corresponds to or consists of amino acids 179 to 192 of the amino acid sequences set forth in SEQ ID NO: 17, 19, 21, or 23, preferably consists of amino acid sequences set forth in SEQ ID NO: 11, 12, or 13.

In one embodiment of this aspect of the invention, the CD81-binding peptide of the invention comprised by the polypeptide is not flanked by naturally occurring HCV sequences. In a preferred embodiment, the CD81-binding peptide of the invention comprised by the polypeptide is not flanked by naturally occurring HCV E1 and/or E2 sequences.

It is another aspect of the present invention to provide a polynucleotide, preferably isolated, coding for the above-described CD81-binding peptides of HCV E2 and variants thereof or for the above described polypeptide comprising the CD81-binding peptide of the invention, which is preferably derived from a naturally occurring HCV genotype. The molecular biology methods applied for obtaining such isolated nucleotide variants are generally known to the person skilled in the art (for standard molecular biology methods see Sambrook et al., Eds., “Molecular Cloning: A Laboratory Manual”, Cold Spring Harbor Laboratory Press, Cold Spring Harbor, N.Y. (1989), which is incorporated herein by reference). For example, RNA can be isolated from HCV infected cells and cDNA generated applying reverse transcription polymerase chain reaction (RT-PCR) using either random primers (e.g., random hexamers of decamers) or primers specific for the generation of the variants of interest. The variants of interest can then be amplified by standard PCR using variant specific primers, which can be designed by the skilled person based on the sequence data available, for example, on the Homepage of the Viral Bioinformatics Resource Center.

The polynucleotide sequence encoding the CD81-binding peptide of the invention may be derived from any HCV genotype as described above. In a preferred embodiment, the polynucleotide sequence encoding the CD81-binding peptide or variants thereof of the present invention is derived from a nucleotide sequence as set forth in SEQ ID NO: 1, 2, 4, 6, or 8. In this context, “derived” refers to the fact that SEQ ID NO: 1, 2, 4, 6, and 8 encode the full-length HCV E2 peptides, and thus, polynucleotides coding for preferred CD81-binding E2 peptides comprise deletions at the 5′ and 3′ ends of the polynucleotide as required by the respectively encoded E2 peptide.

In a preferred embodiment, a polynucleotide as described herein encodes a CD81-binding peptide of HCV E2, which is devoid of HVR1, preferably showing increased CD81 binding when compared to wild type E2. In a preferred embodiment the isolated polynucleotide coding for the preferred embodiments of the CD81-binding peptides or variants thereof are derived from nucleotides 82 to 1092 of the nucleotide sequence set forth in SEQ ID NO: 1 (HCV genotype 1b, isolate: T212) or 8 (HCV genotype 1b, isolate: N2) or degenerate variants thereof, or nucleotides 82 to 1089 of SEQ ID NO: 4 (HCV genotype 1b, isolate: BK), or 6 (HCV genotype 1a, isolate: H77) or degenerate variants thereof. In a preferred embodiment, the codon usage of the isolated polynucleotide coding for the CD81-binding peptide of HCV E2 or a variant thereof according to the present invention is optimized for the respective expression host organism, preferably said polynucleotide is derived from nucleotides 82 to 1092 of the nucleotide sequence set forth in SEQ ID NO: 2 or degenerate variants thereof. In this context, “derived” refers to the fact that SEQ ID NO: 1, 2, 4, 6, and 8 encode the full-length HCV E2 peptides, and thus, polynucleotides coding for preferred CD81-binding E2 peptides comprise deletions at the 5′ and 3′ ends of the polynucleotide as required by the respectively encoded CD81-binding E2 peptide.

In a preferred embodiment, the polynucleotide sequence encoding the CD81-binding peptide of HCV E2 consists of a nucleotide sequence selected from the group of the following nucleotide sequences: nucleotides 82 to 1092, 82 to 1089, 82 to 1086, 82 to 1002, 82 to 999, 82 to 996, 82 to 993, 82 to 906, 82 to 903, 82 to 900, 82 to 897, 82 to 849, 82 to 846, 82 to 843, 82 to 840, 82 to 837, 82 to 834, and 82 to 831 as set forth in SEQ ID NO: 1, 2, or 8, and nucleotides 82 to 1089, 82 to 1086, 82 to 1083, 82 to 999, 82 to 996, 82 to 993, 82 to 990, 82 to 903, 82 to 900, 82 to 897, 82 to 894, 82 to 846, 82 to 843, 82 to 840, 82 to 837, 82 to 834, 82 to 831, and 82 to 828 as set forth in SEQ ID NO: 4 or 6. Polynucleotide variants or variants of the above defined polynucleotide sequences, which encode for a peptide that retains the ability to bind to CD81 are also encompassed by the present invention.

In a another embodiment, the polynucleotide encodes a CD81-binding peptide or variant thereof which is mutated within the HVR1, and preferably exhibits increased CD81 binding. Preferably, the HVR1 is encoded by nucleotides 1 to 81 of the nucleotide sequences as set forth in SEQ ID NO: 1, 2, 4, 6, or 8. For example, polynucleotides encoding CD81-binding peptides of HCV E2 having the following mutations within the HVR1 at 1 to 27 amino acid positions, i.e., at 1, 2, 3, 4, 5, 6, 7, 8, 9, 10, 11, 12, 13, 14, 15, 16, 17, 18, 19, 20, 21, 22, 23, 24, 25, 26, or 27 amino acid positions, compared to amino acids 1 to 27 of the amino acid sequences set forth in SEQ ID NO: 3, 5, 7, or 9, lead to increased CD81-binding and are encompassed by the present invention: amino acids Cys, Phe, Leu, Met, Pro, Trp, or Tyr at amino acid position 1; amino acids Ala, Cys, Asp, Glu, Phe, Gly, His, Ile, Lys, Leu, Met, Asn, Pro, Gln, Arg, Val, Trp, or Tyr at amino acid position 2; amino acids Ala, Asp, Glu, Phe, Gly, Lys, Pro, or Trp at amino acid position 3; amino acids Asp, Glu, Phe, Gly, Lys, Asn, Pro, or Tip at amino acid position 4; amino acids Cys, Asp, Glu, Gly, His, Lys, Asn, Pro, Arg, or Tyr at amino acid position 5; amino acids Cys, Asp, Glu, Phe, His, Ile, Lys, Leu, Met, Asn, Pro, Gln, Arg, Ser, Thr, Val, Tip, or Tyr at amino acid position 6; amino acids Cys, Asp, Phe, Ile, Lys, Leu, Asn, Pro, Thr, Tip, or Tyr at amino acid position 7; amino acids Asp, Phe, Gly, Met, Pro, Trp, or Tyr at amino acid position 8; amino acids Cys, Asp, Phe, Gly, His, Ile, Lys, or Trp at amino acid position 9; amino acids Cys, Asp, Phe, His, Ile, Lys, Leu, Met, Asn, Pro, Gln, Arg, Thr, Val, Tip, or Tyr at amino acid position 10; amino acids Asp, Ile, Leu, Met, Asn, Pro, Thr, Val, or Tip at amino acid position 11; amino acids Cys, Phe, Lys, Met, or Tip at amino acid position 12; amino acids Cys, Asp, Glu, Phe, Gly, His, Lys, Asn, Pro, Gln, Arg, Ser, Tip, or Tyr at amino acid position 13; amino acids Cys, Asp, Glu, Ile, or Pro at amino acid position 14; amino acids Cys, Asp, Glu, Phe, His, Leu, Met, Pro, Trp, or Tyr at amino acid position 15; amino acids Ala, Cys, Asp, Glu, Gly, His, Lys, Met, Asn, Pro, Gln, Arg, Thr, or Tip at amino acid position 16; amino acids Cys, Asp, Glu, Phe, Gly, His, Ile, Lys, Leu, Met, Gln, or Tip at amino acid position 17; amino acids Cys, Glu, Phe, His, Ile, Met, Pro, Gln, Val, Tip, or Tyr at amino acid position 18; amino acids Ala, Cys, Asp, Glu, Gly, His, Lys, Asn, Pro, Gln, Arg, Thr, Val, Tip, or Tyr at amino acid position 19; amino acids Ala, Cys, Asp, Glu, Gly, His, Ile, Lys, Met, Asn, Pro, Gln, Arg, Thr, Val, Tip, or Tyr at amino acid position 20; amino acids Cys, Glu, Phe, His, Ile, Lys, Pro, Val, Tip, or Tyr at amino acid position 21; amino acids Cys, Asp, Glu, Gly, or Asn at amino acid position 22; amino acids Cys, Asp, Glu, Phe, His, Ile, Lys, Leu, Met, Asn, Pro, Gln, Arg, Ser, Thr, Val, Trp, or Tyr at amino acid position 23; amino acids Cys, Asp, Glu, Phe, His, Ile, Lys, Leu, Met, Asn, Thr, Val, Trp, or Tyr at amino acid position 24; amino acids Cys, Asp, Glu, Phe, Gly, Ile, Leu, or Pro at amino acid position 25; amino acids Ala, Cys, Asp, Phe, Gly, Ile, Lys, Leu, Met, Asn, Pro, Ser, Thr, Val, Trp, or Tyr at amino acid position 26, and/or amino acids Ala, Cys, Phe, Gly, Ile, Met, Pro, or Trp at amino acid position 27. In a preferred embodiment, positions 2, 6, 7, 10, 13, 16, 19, 20, 23, and/or 26 or any combination thereof are substituted with the above indicated amino acids for the corresponding amino acid positions. In a more preferred embodiment, positions 2, 6, 7, 16, 19, 20, 23, and/or 26 or any combination thereof are substituted with the above indicated amino acids for the corresponding amino acid positions. The skilled person is well aware of tools to generate mutations in a nucleotide sequence. For example, mutations may be introduced into a polynucleotide by site-directed mutagenesis using polymerase chain reaction (PCR) or commercially available mutagenesis kits such as the QuickChange® Site-Directed Mutagenesis Kit from Stratagene (La Jolla, Calif., USA) or the Transformer™ Site-Directed Mutagenesis Kit from Clontech (Mountain View, Calif., USA).

In a preferred embodiment, the polynucleotide sequence encoding the CD81-binding peptide of HCV E2 is derived from a nucleotide sequence selected from the group of the following nucleotide sequences: nucleotides 1 to 1092, 1 to 1089, 1 to 1086, 1 to 1002, 1 to 999, 1 to 996, 1 to 993, 1 to 906, 1 to 903, 1 to 900, 1 to 897, 1 to 849, 1 to 846, 1 to 843, 1 to 840, 1 to 837, 1 to 834, and 1 to 831 as set forth in SEQ ID NO: 1, 2, or 8, and nucleotides 1 to 1089, 1 to 1086, 1 to 1083, 1 to 999, 1 to 996, 1 to 993, 1 to 990, 1 to 903, 1 to 900, 1 to 897, 1 to 894, 1 to 846, 1 to 843, 1 to 840, 1 to 837, 1 to 834, 1 to 831, and 1 to 828 as set forth in SEQ ID NO: 4 or 6, wherein nucleotides 1 to 81 are mutated or deleted in such a way, that these nucleotides encode an HVR1 that is mutated or deleted in N amino acids, wherein N is any number between 1 and 27, i.e., 1, 2, 3, 4, 5, 6, 7, 8, 9, 10, 11, 12, 13, 14, 15, 16, 17, 18, 19, 20, 21, 22, 23, 24, 25, 26, or 27. Variants of said polynucleotide sequences are also encompassed by the present invention as long as said polynucleotide encodes a peptide capable of binding to CD81, preferably with increased CD81 binding when compared to wild type E2 glycoproteins having a wild type HVR1, preferably compared to an E2 protein having the identical amino acid sequence as the CD81-binding peptide with the exception of HVR1 which is wild type. HVR1 mutations leading to increased CD81 binding are described above.

In an embodiment of this aspect of the present invention, the polynucleotide encoding for the CD81-binding peptide or variant thereof according to the present invention exhibits 60%, 65%, 70%, 80%, 81%, 82%, 83%, 84%, 85%, 86%, 87%, 88%, 89%, 90%, 91%, 92%, 93%, 94%, 95%, 96%, 97%, 98%, 99% sequence similarity, preferably sequence identity over the entire length of the polynucleotide or variant thereof using the best sequence alignment and/or over the region of the best sequence alignment, wherein the best sequence alignment is obtainable with art known tools, e.g., Align, using standard settings, preferably EMBOSS::needle, Matrix: Blosum62, Gap Open 10.0, Gap Extend 0.5, with one of the nucleotide sequences set forth in SEQ ID NO: 1, 2, 4, 6, or 8. Preferably such polynucleotide hybridizes to a polynucleotide encoding a CD81-binding peptide based on the polynucleotide sequence of SEQ ID NO: 1, 2, 4, 6, or 8 under stringent conditions, wherein said stringent conditions comprise hybridization at about 65° C. followed by washing for about 1 hour in 2×SSC buffer at about 65° C., then washing for about 30 minutes in 0.2×SSC buffer at about 65° C.

In another preferred embodiment, the polynucleotide encodes the polypeptide comprising a CD81-binding peptide of the invention. In a preferred embodiment, the polynucleotide encoding the polypeptide comprising a CD81-binding peptide of the invention may include a nucleotide sequence encoding a heterologous protein. In the context of the invention “a nucleotide sequence encoding a heterologous protein” means a nucleotide/polynucleotide encoding a protein which is not derived from HCV. Examples for such heterologous proteins are protein tags as described below. Preferably, the heterologous protein is selected from the group consisting of Interleukin 2 (IL2), Ubiquitin, the antibody fragment crystallizable region (Fc region), Furin cleavage sites, the T helper universal epitope from LTB, and glycoprotein D (gD) of herpes simplex virus (HSV). In a particular preferred embodiment, the heterologous protein is selected from the group consisting of Furin cleavage sites, the T helper universal epitope from the B subunit of the heat labile enterotoxin (LTB), and the gD protein of HSV. The skilled person is well aware of how to obtain such nucleotide sequences, for example, using public databases such as the database provided by the National Center for Biotechnology Information (NCBI; http://www.ncbi.nlm.nih.gov/sites/entrez).

Preferably, a nucleotide sequence encoding a heterologous protein is fused in frame to the 5′- or the 3′-end of the nucleotide encoding the CD81-binding peptide of the present invention.

In another embodiment, the polynucleotide encoding the polypeptide comprising a CD81-binding peptide of the present invention may include a nucleotide sequence encoding an HCV protein other than E2. In the context of the present invention, “a nucleotide sequence encoding an HCV protein other than E2” means any other nucleotide sequence encoding for an HCV protein except of the nucleotide sequence encoding the envelope glycoprotein E2, i.e., capsid, E1, NS2, NS3, NS4A, NS4B, NS5A, NS5B. In a preferred embodiment, the nucleotide sequence encoding an HCV protein other than E2 is a nucleotide sequence encoding the envelope glycoprotein E1. The E1 sequence may be derived from any of the above described HCV genotypes, strains, or isolates. In a preferred embodiment, the E1 sequence is derived from HCV isolates T212, BK, H77, or N2. In a more preferred embodiment, the E1 nucleotide sequence is derived from or consists of one of the nucleotide sequences set forth in SEQ ID NO: 16, 18, 20, and 22 or degenerate variants thereof. The E1 nucleotide sequence may encode the full-length E1 protein or fragments thereof. In a preferred embodiment the E1 nucleotide sequence encodes the 14 carboxy-terminal amino acids of the E1 protein. In a preferred embodiment, the E1 protein has an amino acid sequence as set forth in SEQ ID NO: 17, 19, 21, or 23 such that the 14 carboxy terminal amino acids have a sequence as set forth in SEQ ID NO: 11, 12, or 13.

Preferably, the nucleotide sequence encoding for an HCV protein other than E2 is fused in frame to the 5′- or the 3′-end of the nucleotide sequence encoding for the CD81-binding peptide of the invention, more preferably the 5′-end.

In one embodiment of this aspect of the invention, the nucleotide sequence encoding the CD81-binding peptide of the invention is not flanked by naturally occurring HCV nucleotide sequences. In a preferred embodiment, the nucleotide sequence encoding the CD81-binding peptide of the invention is not flanked by naturally occurring HCV E1 and/or E2 sequences.

In another embodiment, the polynucleotide of the present invention includes functional sequences that lead to secretion of the encoded peptide or polypeptide. The signal sequence variant usually encodes a signal peptide comprised of hydrophobic amino acids which direct the secretion of the protein from the cell. Preferably, the secretion signal peptide is derived from the HCV envelope protein E1, which is located to the carboxy terminus of E1. In a preferred embodiment, the E1 signal sequence corresponds to or consists of amino acids 179 to 192 of SEQ ID NO: 17, 19, 21, or 23 and may be elongated by corresponding amino terminal amino acids by about 1, 2, 3, 4, 5, 6, 7, 8, 9, or 10 amino acids. In a preferred embodiment, the E1 secretion signal sequence consists of amino acids as set forth in SEQ ID NO: 11, 12, or 13. In another preferred embodiment, the secretion signal peptide is derived from the tissue plasminogen activator (tPA), preferably the tPA signal sequence corresponds to or consists of the amino acid sequence as set forth in SEQ ID NO: 15 and is encoded by a nucleotide sequence as set forth in SEQ ID NO: 14 or a degenerate variant thereof. Thus, in a preferred embodiment, the polynucleotide of the present invention comprises the E1 or the tPA signal sequence fused in frame to the 5′-end of the nucleotide sequence encoding the CD81-binding peptide according to the present invention or encoding the polypeptide comprising the CD81-binding peptide of the invention.

The present invention also comprises the proteins encoded by the above described polynucleotide.

In another aspect, the present invention provides an expression cassette comprising the above defined polynucleotide, and one or more polynucleotides selected from the group consisting of promoter, enhancer, a ribosomal binding site, a Kozak sequence, a nucleotide sequence encoding a heterologous protein, a nucleotide sequence encoding an epitope-, peptide- or protein-tag, and a polyadenylation signal.

Promoters are genetic elements that are recognized by an RNA polymerase and mediate transcription of downstream regions. Preferred promoters are strong promoters that provide for increased levels of transcription. Examples of strong promoters are the immediate early human cytomegalovirus promoter (CMV) and CMV with intron A (Chapman et al., 1991). Additional examples for promoters include naturally occurring promoters such as the EF1 alpha promoter, the murine CMV promoter, the African Green Monkey CMV promoter, Rous Sarcoma virus promoter, SV40 early/late promoters, and the β-actin promoter. Furthermore, artificial promoters such as a synthetic muscle specific promoter and a chimeric muscle-specific/CMV promoter are also applicable in the present invention (Li et al., 1999; Hagstrom et al., 2000).

Furthermore, regulated expression is also encompassed by this aspect of the present invention. Regulated expression of a gene, cDNA, or nucleotide sequence in general, may be achieved by inducible promoters. The activity of such promoters may be triggered by either chemical or physical factors. For instance, chemically regulated promoters include promoters whose transcriptional activity is regulated by the presence or absence of alcohol, tetracycline, steroids, metal or other compounds. For example, a metallothionein promoter (Makarov et al., 1994), a tetracycline inducible promoter system (Baron et al., 1997), or a cell cycle regulated promoter system may be used.

In a preferred embodiment, regulated expression of the transgene inserted in Ad vectors can be obtained by the insertion of a Tet operator sequence linked to a promoter (i.e., human CMV promoter) immediately downstream of the transcription start site and before the translation start site. The resulting vector encoding the CD81-binding peptide is produced in cells expressing the Tet repressor protein (i.e., the HEK 293 cell line expressing the Tet repressor protein). The same regulated expression can be obtained by using any promoter of viral or cellular origin associated with Tet or lac operator sequences and the corresponding vector being produced in suitable cell lines expressing Tet repressor or lac repressor proteins.

An enhancer region increases transcription. Examples of enhancer regions include the CMV enhancer and the SV40 enhancer (Hitt et al., 1995; Xu et al., 2001). An enhancer region can be associated with a promoter.

The ribosomal binding site is located at or near the initiation codon. Examples of preferred ribosomal binding sites include CCACCAUGG, CCGCCAUGG, and ACCAUGG, where AUG is the initiation codon. A preferred Kozak sequence is GCCACC preceding the AUG.

In this aspect of the present invention, “a nucleotide sequence encoding a heterologous protein” means any nucleotide sequence not encoding for an HCV protein. Such nucleotide sequences may, for example, encode selection markers and thereby, e.g., confer antibiotic resistance. An example for a selection marker is a neomycin resistance gene. Preferably, such heterologous nucleotide sequences are linked to the polynucleotide encoding the CD81-binding peptide or the polynucleotide encoding the polypeptide comprising the CD81-binding peptide of the invention, for example, by an internal ribosomal entry site (IRES), which results in expression of two separate, non-fused proteins, i.e., the CD81-binding peptide or the polypeptide comprising the CD81-binding peptide of the invention and the heterologous protein.

Epitope-, peptide-, or protein-tags facilitate purification of polypeptide variants of interest. Such epitope-, peptide-, or protein-tags include, but are not limited to, hemagglutinin- (HA-), FLAG-, myc-tag, poly-His-tag, glutathione-S-transferase- (GST-), maltose-binding-protein-(MBP-), NusA-, and thioredoxin-tag, or fluorescent protein-tags such as (enhanced) green fluorescent protein ((E)GFP), (enhanced) yellow fluorescent protein ((E)YFP), red fluorescent protein (RFP) derived from Discosoma species (DsRed) or monomeric (mRFP), cyan fluorescence protein (CFP), and the like. In a preferred embodiment, the epitope-, peptide-, or protein-tags can be cleaved off the polypeptide variant of interest, for example, using a protease such as thrombin, Factor Xa, PreScission, TEV protease, and the like. The recognition sites for such proteases are well known to the person skilled in the art.

The polyadenylation signal is responsible for cleaving the transcribed RNA and the addition of a poly(A)tail to the RNA. The polyadenylation signal in higher eukaryotes contains an AAUAAA sequence about 11 to 30 nucleotides from the polyadenylation addition site. The poly(A)tail is important for the mRNA processing. Polyadenylation signals that can be used as part of the expression cassette include the minimal rabbit β-globin polyadenylation signal and the bovine growth hormone polyadenylation signal (BGH) (Xu et al., 2001; U.S. Pat. No. 5,122,458). Additional examples include the Synthetic Polyadenylation Signal (SPA) and SV40 polyadenylation signal. The SPA sequence is as follows: AAUAAAAGAU-CUUUAUUUUCAUUAGAUCUGUGUGUUGGUUUUUUGUGUG (SEQ ID NO: 10)

In another aspect, the present invention relates to a recombinant vector comprising the polynucleotide encoding the CD81-binding peptide of the invention or the polypeptide comprising the CD81-binding peptide of the invention, or the expression cassette described above.

Thus, in one aspect, the present invention relates to a recombinant vector comprising (i) a polynucleotide encoding a CD81-binding peptide of hepatitis C virus (HCV) envelope protein E2, which is devoid of the hypervariable region 1 (HVR1), i.e., which lacks the 27 amino terminal amino acids, or which is mutated within the HVR1, or (ii) a polynucleotide encoding a polypeptide comprising said CD81-binding peptide of the invention, or (iii) an expression cassette comprising said polynucleotide, wherein the further and preferred embodiments of the CD81-binding peptide, the polypeptide, the polynucleotide, and the expression cassette are as described above.

The person skilled in the art is well aware of techniques used for the incorporation of polynucleotide sequences of interest into vectors (also see Sambrook et al., 1989). Such vectors include any vectors known to the skilled person including plasmid vectors, cosmid vectors, phage vectors such as bacteriophage lambda, viral vectors such as adenovirus (Ad) vectors (e.g., non-replicating Ad5, Ad11, Ad26, Ad35, Ad49, ChAd3, ChAd4, ChAd5, ChAd6, ChAd7, ChAd8, ChAd9, ChAd10, ChAd11, ChAd16, ChAd17, ChAd19, ChAd20, ChAd22, ChAd24, ChAd26, ChAd30, ChAd31, ChAd37, ChAd38, ChAd44, ChAd63, ChAd82, ChAd55, ChAd73, ChAd83, ChAd146, ChAd147, PanAd1, PanAd2, and PanAd3 vectors or replication-competent Ad4 and Ad7 vectors), adeno-associated virus (AAV) vectors (e.g., AAV type 5), alphavirus vectors (e.g., Venezuelan equine encephalitis virus (VEE), sindbis virus (SIN), semliki forest virus (SFV), and VEE-SIN chimeras), herpes virus vectors, measles virus vectors, pox virus vectors (e.g., vaccinia virus, modified vaccinia virus Ankara (MVA), NYVAC (derived from the Copenhagen strain of vaccinia), and avipox vectors: canarypox (ALVAC) and fowlpox (FPV) vectors), and vesicular stomatitis virus vectors (for a review on viral vectors see Robert-Guroff, 2007), baculoviral vectors, viral-like particles, bacterial spores, or artificial chromosome vectors such as bacterial artificial chromosomes (BAC), yeast artificial chromosomes (YAC), or P1 artificial chromosomes (PAC). Said vectors may be expression vectors suitable for prokaryotic or eukaryotic expression. Said vectors may include an origin of replication (ori), a multiple cloning site, and regulatory sequences such as promoter (constitutive or inducible), transcription initiation site, ribosomal binding site, transcription termination site, polyadenylation signal, and selection marker such as antibiotic resistance or auxotrophic marker based on complementation of a mutation or deletion. In one embodiment the polynucleotide sequence encoding for the CD81-hinging peptide of the invention is operably linked to the regulatory sequences. If the expression cassette as described above is introduced into a vector, it is not required that said vector includes the regulatory elements that are present in the expression cassette. In this case, the vector may include elements such as a selectable marker, an origin of replication, homologous recombination regions, and/or convenient restriction sites necessary for cloning, amplification, or selection purposes. In a preferred embodiment, the vector is selected from the group consisting of a viral vector, a viral-like particle, a bacterial spore, a bacteriophage vector, and a plasmid. In an even more preferred embodiment, the vector is a viral vector selected from the group consisting of an adenoviral vector, adeno-associated virus vector, MVA (modified vaccinia virus Ankara), ALVAC, NYVAC, fowlpox virus vector, alphavirus vector or measles virus vector. In an even more preferred embodiment, the viral vector is selected form adenovirus and MVA, and in a most preferred embodiment, the viral vector is selected from the group consisting of replication-defective ChAd55, replication-defective ChAd73, replication-defective ChAd83, replication-defective ChAd146, replication-defective ChAd147, replication-defective PanAd1, replication-defective PanAd2, and replication-defective PanAd3 vectors.

In another aspect, the present invention provides a recombinant host cell comprising the polynucleotide encoding a CD81-binding peptide of the present invention, the expression cassette comprising the polynucleotide encoding the CD81-binding peptide of the present invention, or the vector comprising the polynucleotide or the expression cassette according to the present invention. The recombinant host cells may be prokaryotic cells such as archea and bacterial cells or eukaryotic cells such as yeast, plant, insect, or mammalian cells. In one embodiment the host cell is a bacterial cell such as an E. coli cell. The person skilled in the art is well aware of methods for introducing said isolated polynucleotide or said recombinant vector into said host cell. For example, bacterial cells can be readily transformed using, for example, chemical transformation, e.g., the calcium chloride method, or electroporation. In another embodiment, the recombinant host cell is a eukaryotic cell. Yeast cells may be transformed, for example, using the lithium acetate transformation method or electroporation. Other eukaryotic cells can be transfected, for example, using commercially available liposome-based transfection kits such as Lipofectamine™ (Invitrogen), commercially available lipid-based transfection kits such as Fugene (Roche Diagnostics), polyethylene glycol-based transfection, calcium phosphate precipitation, gene gun (biolistic), electroporation, or viral infection. In a preferred embodiment of the present invention, the recombinant host cell expresses the CD81-binding peptides of HCV E2 of the invention. In an even more preferred embodiment, said expression leads to soluble CD81-binding peptides according to the present invention. These CD81-binding peptides can be purified using protein purification methods well known to the person skilled in the art, optionally taking advantage of the above-mentioned epitope-, peptide-, or protein-tags.

In another aspect, the present invention provides a composition comprising a CD81-binding peptide according to the present invention, a polypeptide comprising the CD81-binding peptide according to the present invention, a polynucleotide encoding a CD81-binding peptide according to the present invention, an expression cassette according to the present invention as defined above, or a vector comprising a polynucleotide or the expression cassette according to the present invention, and an adjuvant. Examples of adjuvants that may be used in the context of the composition according to the present invention are gel-like precipitates of aluminum hydroxide (alum); AlPO₄; alhydrogel; bacterial products from the outer membrane of Gram-negative bacteria, in particular monophosphoryl lipid A (MPLA), lipopoly-saccharides (LPS), muramyl dipeptides and derivatives thereof; Freund's incomplete adjuvant; liposomes, in particular neutral liposomes, liposomes containing the composition and optionally cytokines; non-ionic block copolymers; ISCOMATRIX adjuvant (Drane et al., 2007); unmethylated DNA comprising CpG dinucleotides (CpG motif), in particular CpG ODN with a phosphorothioate (PTO) backbone (CpG PTO ODN) or phosphodiester (PO) backbone (CpG PO ODN); synthetic lipopeptide derivatives, in particular Pam₃Cys; lipoarabinomannan; peptidoglycan; zymosan; heat shock proteins (HSP), in particular HSP 70; dsRNA and synthetic derivatives thereof, in particular Poly I:poly C; polycationic peptides, in particular poly-L-arginine; taxol; fibronectin; flagellin; imidazoquinoline; cytokines with adjuvant activity, in particular GM-CSF, interleukin- (IL-)2, IL-6, IL-7, IL-18, type I and II interferons, in particular interferon-gamma, TNF-alpha; 25-dihydroxyvitamin D3 (calcitriol); and synthetic oligopeptides, in particular MHCII-presented peptides. Non-ionic block polymers containing polyoxyethylene (POE) and polyoxypropylene (POP), such as POE-POP-POE block copolymers may be used as an adjuvant (Newman et al., 1998). This type of adjuvant is particularly useful for compositions comprising nucleic acids as active ingredient.

Activation of specific receptors stimulate an immune response. Such receptors are known to the skilled artisan and comprise, for example, cytokine receptors, in particular type I cytokine receptors, type II cytokine receptors, TNF receptors; and vitamin D receptor acting as transcription factor; and the Toll-like receptors 1 (TLR1), TLR-2, TLR 3, TLR4, TLR5, TLR-6, TLR7, and TLR9. Agonists to such receptors have adjuvant activity, i.e., are immuno-stimulatory. In a preferred embodiment, the adjuvant of the composition of the present invention may be one or more Toll-like receptor agonists. In a more preferred embodiment, the adjuvant is a Toll-like receptor 4 agonist. In a particular preferred embodiment, the adjuvant is a Toll-like receptor 9 agonist, preferably being encoded by the nucleotide sequence set forth in SEQ ID NO: 27.

In another aspect, the present invention provides a pharmaceutical composition or vaccine comprising a CD81-binding peptide according to the present invention, a polypeptide comprising a CD81-binding peptide according to the present invention, a polynucleotide encoding a CD81-binding peptide according to the present invention or a polypeptide comprising the CD81-binding peptide of the present invention, an expression cassette according to the present invention as defined above, a vector comprising a polynucleotide or the expression cassette according to the present invention, or a composition according to the present invention, and a pharmaceutically acceptable excipient, carrier or diluent. In one embodiment, the pharmaceutical composition or vaccine of the present invention comprises a protein comprising the CD81-binding peptide according to the present invention, a pharmaceutically acceptable excipient, carrier, or diluent, and optionally an adjuvant. In another embodiment, the pharmaceutical composition or vaccine of the present invention comprises the CD81-binding peptide of the present invention, a pharmaceutically acceptable excipient, carrier, or diluent, and optionally an adjuvant. In a preferred embodiment, the pharmaceutical composition or vaccine of the present invention comprises a polynucleotide encoding a CD81-binding peptide of the present invention, a pharmaceutically acceptable excipient, carrier, or diluent, and optionally an adjuvant. In another embodiment, the pharmaceutical composition or vaccine of the present invention comprises the expression cassette according to the present invention, a pharmaceutically acceptable excipient, carrier, or diluent, and optionally an adjuvant. In a preferred embodiment, the pharmaceutical composition or vaccine of the present invention comprises a vector comprising the polynucleotide encoding the CD81-binding peptide according to the present invention or the expression cassette according to the present invention, a pharmaceutically acceptable excipient, carrier, or diluent, and optionally an adjuvant. In a more preferred embodiment, said vector is a viral vector, a viral-like particle, a bacterial spore, a bacteriophage vector, or a plasmid. In an even more preferred embodiment, the vector is a viral vector such as adenovirus (Ad) vectors (e.g., non-replicating Ad5, Ad11, Ad26, Ad35, Ad49, ChAd3, ChAd4, ChAd5, ChAd6, ChAd7, ChAd8, ChAd9, ChAd10, ChAd11, ChAd16, ChAd17, ChAd19, ChAd20, ChAd22, ChAd24, ChAd26, ChAd30, ChAd31, ChAd37, ChAd38, ChAd44, ChAd63, ChAd82, ChAd55, ChAd73, ChAd83, ChAd146, ChAd147, PanAd1, PanAd2, and PanAd3 vectors or replication-competent Ad4 and Ad7 vectors), adeno-associated virus (AAV) vectors (e.g., AAV type 5), alphavirus vectors (e.g., Venezuelan equine encephalitis virus (VEE), sindbis virus (SIN), semliki forest virus (SFV), and VEE-SIN chimeras), herpes virus vectors, measles virus vectors, pox virus vectors (e.g., vaccinia virus, modified vaccinia virus Ankara (MVA), NYVAC (derived from the Copenhagen strain of vaccinia), and avipox vectors: canarypox (ALVAC) and fowlpox (FPV) vectors), and vesicular stomatitis virus vectors. In a more preferred embodiment, the viral vector is selected from the group consisting of an adenoviral vector, adeno-associated virus vector, MVA, ALVAC, NYVAC, fowlpox virus vector, alphavirus vector, or measles virus vector. In an even more preferred embodiment, said vector comprised by the pharmaceutical composition or vaccine of the present invention is an adenoviral vector or MVA, and in a most preferred embodiment, the viral vector comprised by the pharmaceutical composition is selected from the group consisting of replication-defective ChAd55, replication-defective ChAd73, replication-defective ChAd83, replication-defective ChAd146, replication-defective ChAd147, replication-defective PanAd1, replication-defective PanAd2, and replication-defective PanAd3 vectors. In another preferred embodiment, the pharmaceutical composition or vaccine of the present invention comprises the composition of the present invention and a pharmaceutically acceptable excipient, carrier, or diluent.

In a preferred embodiment, the CD81-binding peptide of the present invention is used as an immunogen in the composition, pharmaceutical composition or vaccine of the present invention. The immunogen may be present as protein in the composition, pharmaceutical composition or vaccine of the present invention, i.e., a protein comprising the CD81-binding peptide of the invention or the CD81-binding peptide of the present invention itself, or the immunogen may be encoded by a nucleic acid present in the composition, pharmaceutical composition or vaccine of the present invention, such as in the case of nucleic acid vaccines, e.g., DNA vaccines.

In a preferred embodiment, the composition, pharmaceutical composition or vaccine according to the present invention is used for the treatment of an existing HCV infection in a patient. In this embodiment, the composition, pharmaceutical composition or vaccine of the present invention enhances or induces an immune response directed against HCV in a mammal/patient, preferably a human patient, that is infected with HCV. For a patient infected with HCV, an effective amount of the pharmaceutical composition or vaccine of the present invention used for the treatment of the HCV infection is sufficient to achieve one or more of the following effects: prevent infection, reduce the ability of HCV to replicate, reduce HCV load, increase viral clearance, and increase the HCV specific immune response, such as the cell mediated immune response. In another preferred embodiment, the composition, pharmaceutical composition or vaccine according to the present invention is used for prophylactic purposes to protect a mammal/patient, preferably a human patient, from an infection with HCV by inducing an immune response directed against HCV, preferably inducing immunological memory. For a patient not infected with HCV, an effective amount of the pharmaceutical composition or vaccine of the present invention for prophylactic purposes is sufficient to achieve one or more of the following effects: an increased ability to produce one or more components of an HCV specific immune response, such as a humoral and cell mediated immune response, to an HCV infection, a reduced susceptibility to HCV infection, and a reduced ability of the infecting virus to establish persistent infection for chronic disease.

The composition, pharmaceutical composition or vaccine of the present invention can be formulated and administered to a patient using the guidance provided herein along with techniques well known in the art. Guidelines for pharmaceutical administration in general are provided in, for example, Modern Vaccinology, ed. Kurstak, Plenum Med. Co. 1994; Remington's Pharmaceutical Sciences 18^{th} Edition, ed. Gennaro, Mack Publishing, 1990; and Modern Pharmaceutics 2^{nd} Edition, eds. Banker and Rhodes, Marcel Dekker, Inc., 1999, each of which are hereby incorporated by reference herein.

The composition, pharmaceutical composition or vaccine according to the present invention is typically prepared as injectables, either as liquid solutions or suspensions. Solid forms suitable for solution or suspension in liquid vehicles prior to injection may also be prepared. The pharmaceutical composition or vaccine according to the present invention will comprise a therapeutically effective amount of the CD81-binding peptide of HCV E2 according to the present invention, a polynucleotide encoding the CD81-binding peptide according to the present invention, the expression cassette according to the present invention, or the vector according to the present invention.

Solid administration forms of the composition, pharmaceutical composition or vaccine of the present invention may contain excipients such as microcrystalline cellulose, lactose, sodium citrate, calcium carbonate, dibasic calcium phosphate, glycine, and starch (preferably corn, potato, or tapioca starch), disintegrants such as sodium starch glycolate, croscarmellose sodium, and certain complex silicates, and granulation binders such as polyvinylpyrrolidone, hydroxypropylmethyl cellulose (HPMC), hydroxypropylcellulose (HPC), sucrose, gelatin, and acacia. Additionally, lubricating agents such as magnesium stearate, stearic acid, glyceryl behenate, and talc may be included. Solid compositions of a similar type may also be employed as fillers in gelatin capsules. Preferred excipients in this regard include lactose, starch, a cellulose, milk sugar or high molecular weight polyethylene glycols.

The composition, pharmaceutical composition or vaccine of the present invention suitable for parenteral administration is best used in the form of a sterile aqueous solution which may contain other substances, for example, enough salts or glucose to make the solution isotonic with blood. The aqueous solutions should be suitably buffered (preferably to a pH of from 3 to 9), if necessary. In a preferred embodiment, the pharmaceutical composition of the present invention is administered by a parenteral route, preferably intravenous, intraperitoneal, subcutaneous, intramuscular, intradermal, or by impression through the skin. The most preferred route is intramuscular.

The composition, pharmaceutical composition or vaccine of the present invention suitable for intranasal administration and administration by inhalation is best delivered in the form of a dry powder inhaler or an aerosol spray from a pressurized container, pump, spray or nebulizer with the use of a suitable propellant, e.g., dichlorodifluoromethane, trichlorofluoromethane, dichlorotetrafluoroethane, a hydrofluoroalkane such as 1,1,1,2-tetrafluoroethane (HFA 134A™) or 1,1,1,2,3,3,3-heptafluoropropane (HFA 227EA™), carbon dioxide, or another suitable gas. The pressurized container, pump, spray or nebulizer may contain a solution or suspension of the active compound, e.g., using a mixture of ethanol and the propellant as the solvent, which may additionally contain a lubricant, e.g., sorbitan trioleate.

In another aspect, the present invention provides a CD81-binding peptide according to the present invention, a polypeptide comprising the CD81-binding peptide according to the present invention, a polynucleotide encoding the CD81-binding peptide according to the present invention or a polypeptide comprising the CD81-binding peptide of the invention, an expression vector according to the present invention, a vector according to the present invention, a composition according to the present invention, or a pharmaceutical composition or vaccine of the present invention for induction of an immune response against HCV in a mammal. In a preferred embodiment, said mammal is a human. In a preferred embodiment, the immune response is therapeutic and/or prophylactic. A therapeutic immune response is directed against an existing HCV infection and shall reduce the ability of HCV to infect, to replicate, reduce the HCV load, and increase viral clearance. A prophylactic immune response shall reduce the susceptibility to a future HCV infection and/or shall reduce the ability of the infecting virus to establish persistent infection for chronic disease. In a preferred embodiment, the immune response, preferably therapeutic and/or prophylactic, is directed against two or more, i.e., 3, 4, 5, 6, 7, 8, 9, 10 or more, different HCV isolates. In an even more preferred embodiment, the immune response is directed against two or more, i.e., 3, 4, 5, 6, 7 or more, different HCV genotypes. Thus, the immune response in this preferred embodiment exhibits broad-specificity for HCV genotypes. In a preferred embodiment, the immune response is directed against heterologous HCV infection.

In another aspect, the present invention provides a method for inducing an immune response in a mammal against HCV comprising administering to said mammal the CD81-binding peptide according to the present invention, the polypeptide comprising the CD81-binding peptide according to the present invention, the polynucleotide encoding the CD81-binding peptide according to the present invention or the polypeptide comprising the CD81-binding peptide of the invention, the expression cassette according to the present invention, the vector according to the present invention, the composition according to the present invention, or the pharmaceutical composition or vaccine according to the present invention in an amount effective to generate an immune response. In one embodiment, the present invention provides a method for inducing an immune response in a mammal against HCV comprising administering to said mammal the composition, or pharmaceutical composition or vaccine of the present invention in an amount effective to induce an immune response. In another embodiment, the present invention relates to the use of the CD81-binding peptide according to the present invention, the polypeptide comprising the CD81-binding peptide according to the present invention, the polynucleotide encoding the CD81-binding peptide according to the present invention or the polypeptide comprising the CD81-binding peptide of the invention, the expression cassette according to the present invention, the vector according to the present invention, or the composition according to the present invention for the manufacture of a pharmaceutical composition or vaccine for inducing an immune response in a mammal against HCV.

In a preferred embodiment of the method or use of the present invention, the mammal is a human. In another preferred embodiment of the method or use of the present invention, the immune response is therapeutic and/or prophylactic. A therapeutic immune response is directed against an existing HCV infection and shall reduce the ability of HCV to infect, to replicate, or it shall reduce the HCV load, or increase viral clearance. A prophylactic immune response shall reduce the susceptibility to a future HCV infection and/or shall reduce the ability of the infecting virus to establish persistent infection for chronic disease. In a preferred embodiment, the method or use of the present invention is applied for the treatment of an existing HCV infection in a patient. In this embodiment, the method or use of the present invention results in enhancing or inducing an immune response directed against HCV in a mammal/patient, preferably a human patient, that is infected with HCV. For a patient infected with HCV, an effective amount used for the treatment of the HCV infection is sufficient to achieve one or more of the following effects: reduce the ability of HCV to infect, to replicate, or reduce HCV load, or increase viral clearance, and increase the HCV specific immune response, such as the humoral and cell mediated immune response. In another preferred embodiment, the method or use according to the present invention results in a prophylactic effect to protect a mammal/patient, preferably a human patient, from an infection with HCV by inducing an immune response directed against HCV. For a patient not infected with HCV, an effective amount of for prophylactic purposes is sufficient to achieve one or more of the following effects: an increased ability to produce one or more components of an HCV specific immune response, such as a humoral and a cell mediated immune response to an HCV infection, a reduced susceptibility to HCV infection, and a reduced ability of the infecting virus to establish persistent infection for chronic disease.

In another preferred embodiment of the method or use of the present invention, the immune response is directed against two or more, i.e., 3, 4, 5, 6, 7, 8, 9, 10, or more, different HCV isolates. In an even more preferred embodiment, the immune response is directed against two or more, i.e., 3, 4, 5, 6, 7 or more, different HCV genotypes. Thus, the immune response in this preferred embodiment exhibits broad-specificity for HCV genotypes. In a preferred embodiment of the method or use of the present invention, the immune response is directed against heterologous HCV infection.

In another preferred embodiment of the method or use of the present invention, at least one booster dose comprising the CD81-binding peptide according to the present invention, the polypeptide comprising the CD81-binding peptide according to the present invention, the polynucleotide encoding the CD81-binding peptide according to the present invention or the polypeptide comprising the CD81-binding peptide of the invention, the expression cassette according to the present invention, the vector according to the present invention, the composition according to the present invention, or the pharmaceutical composition or vaccine according to the present invention is administered to the mammal in an amount effective to enhance the immune response. In a preferred embodiment of the method or use of the present invention, at least one booster dose comprising the composition, pharmaceutical composition or vaccine of the present invention is administered to the mammal in an amount effective to enhance the immune response.

In one embodiment of the method or use of the present invention, the vector for inducing/generating the immune response (priming) against HCV in a mammal and the vector for enhancing the immune response (boosting) against HCV in a mammal are the same. In another embodiment, the vector for priming against HCV in a mammal and the vector for boosting against HCV in a mammal are different. Thus, in a preferred embodiment of the method or use of the present invention, a heterologous prime-boost administration regimen is applied. Heterologous prime-boost is a mixed modality involving the use of one type of vector for priming and another type of vector for boosting. The heterologous prime-boost can involve related vectors such as vectors based on different adenovirus serotypes and more distantly related viruses such as adenovirus and fowlpox virus. In a preferred embodiment, the heterologous prime-boost involves tow different serologically non cross-reacting adenovirus vectors.

In a preferred embodiment of the method or use of the present invention, the vector for inducing/generating the immune response is selected from the group consisting of adenovirus (Ad) vectors (e.g., non-replicating Ad5, Ad11, Ad26, Ad35, Ad49, ChAd3, ChAd4, ChAd5, ChAd6, ChAd7, ChAd8, ChAd9, ChAd10, ChAd11, ChAd16, ChAd17, ChAd19, ChAd20, ChAd22, ChAd24, ChAd26, ChAd30, ChAd31, ChAd37, ChAd38, ChAd44, ChAd63, ChAd82, ChAd55, ChAd73, ChAd83, ChAd146, ChAd147, PanAd1, PanAd2, and PanAd3 vectors or replication-competent Ad4 and Ad7 vectors), adeno-associated virus (AAV) vectors (e.g., AAV type 5), alphavirus vectors (e.g., Venezuelan equine encephalitis virus (VEE), sindbis virus (SIN), semliki forest virus (SFV), and VEE-SIN chimeras), herpes virus vectors, measles virus vectors, pox virus vectors (e.g., vaccinia virus, modified vaccinia virus Ankara (MVA), NYVAC (derived from the Copenhagen strain of vaccinia), and avipox vectors: canarypox (ALVAC) and fowlpox (FPV) vectors), and vesicular stomatitis virus vectors, and the vector for enhancing the immune response is selected from the group consisting of adenovirus (Ad) vectors (e.g., non-replicating Ad5, Ad11, Ad26, Ad35, Ad49 ChAd3, ChAd4, ChAd5, ChAd6, ChAd7, ChAd8, ChAd9, ChAd10, ChAd11, ChAd16, ChAd17, ChAd19, ChAd20, ChAd22, ChAd24, ChAd26, ChAd30, ChAd31, ChAd37, ChAd38, ChAd44, ChAd63, ChAd82, and ChAd55, ChAd73, ChAd83, ChAd146, ChAd147, PanAd1, PanAd2, and PanAd3 vectors or replication-competent Ad4 and Ad7 vectors), adeno-associated virus (AAV) vectors (e.g., AAV type 5), alphavirus vectors (e.g., Venezuelan equine encephalitis virus (VEE), sindbis virus (SIN), semliki forest virus (SFV), and VEE-SIN chimeras), herpes virus vectors, measles virus vectors, pox virus vectors (e.g., vaccinia virus, modified vaccinia virus Ankara (MVA), NYVAC (derived from the Copenhagen strain of vaccinia), and avipox vectors: canarypox (ALVAC) and fowlpox (FPV) vectors), and vesicular stomatitis virus vectors. In one embodiment, the vectors for priming and boosting are the same. In another embodiment, the vectors for priming and boosting are different. In a preferred embodiment, the vectors for priming and boosting are serologically different, non-cross-reacting adenovirus vectors.

The length of time between priming and boosting typically varies from about four months to a year, but other time frames may be used. In one embodiment, the interval between prime and boost is for a period of at least 6 months. In another embodiment the interval between prime and boost is for a period of at least 3 months. Priming may involve multiple priming steps with one type of vector, such as 2, 3, or 4 primings.

For compositions, pharmaceutical compositions or vaccines according to the present invention comprising viral vectors, i.e., adenovirus (Ad) vectors (e.g., non-replicating Ad5, Ad11, Ad26, Ad35, Ad49, ChAd3, ChAd4, ChAd5, ChAd6, ChAd7, ChAd8, ChAd9, ChAd10, ChAd11, ChAd16, ChAd17, ChAd19, ChAd20, ChAd22, ChAd24, ChAd26, ChAd30, ChAd31, ChAd37, ChAd38, ChAd44, ChAd63, ChAd82, and ChAd55, ChAd73, ChAd83, ChAd146, ChAd147, PanAd1, PanAd2, and PanAd3 vectors or replication-competent Ad4 and Ad7 vectors), adeno-associated virus (AAV) vectors (e.g., AAV type 5), alphavirus vectors (e.g., Venezuelan equine encephalitis virus (VEE), sindbis virus (SIN), semliki forest virus (SFV), and VEE-SIN chimeras), herpes virus vectors, measles virus vectors, pox virus vectors (e.g., vaccinia virus, modified vaccinia virus Ankara (MVA), NYVAC (derived from the Copenhagen strain of vaccinia), and avipox vectors: canarypox (ALVAC) and fowlpox (FPV) vectors), and vesicular stomatitis virus vectors, the amount of viral particles in the composition, or pharmaceutical composition or vaccine composition to be administered to a patient will depend on the strength of the transcriptional and translational promoters used and on the immunogenicity of the expressed gene product. In general, an immunologically or prophylactically effective dose of 1×10⁷ to 1×10¹² viral particles (i.e., 1×10⁷, 2×10⁷, 3×10⁷, 5×10⁷, 1×10⁸, 2×10⁸, 3×10⁸, 5×10⁸, or 1×10⁹, 2×10⁹, 3×10⁹, 5×10⁹) and preferably about 1×10¹⁰ to 1×10¹¹ particles are administered. For adenovirus vectors an immunologically and/or prophylactically effective dose is preferably 1×10⁸ to 1×10¹¹ viral particles (i.e., 1×10⁸, 5×10⁸, 1×10⁹, 5×10⁹, 1×10¹⁰, 2.5×10¹⁰, 5×10¹⁰).

Preferably, administration is performed directly into the muscle tissue. Subcutaneous injection, intradermal introduction, impression through the skin, and other modes of administration such as intraperitoneal, intravenous, or inhalation delivery are also contemplated. The exact amount of active ingredient, i.e., immunogen or nucleic acid encoding the immunogen, to be administered will vary depending on the subject being treated, the age and general condition of the individual to be treated, the capacity of the individual's immune system to synthesize antibodies, the degree of protection desired, the severity of the condition to be treated, the particular CD81-binding peptide of HCV E2 selected and its mode of administration, among other factors. An appropriate effective amount can be readily determined by one of skill in the art.

For compositions, or pharmaceutical compositions or vaccines according to the present invention comprising the CD81-binding peptide of HCV E2, i.e., the immunogen, as a protein the dose for administration to a large mammal, such as a primate, for example, a baboon, chimpanzee, or human, may be approximately 0.1 μg to about 5.0 mg per dose, or any amount between the stated ranges, such as 0.5 to about 1.0 mg, 1 μg to about 500 μg, 2.5 μg to about 250 μg, 4 μg to about 200 μg, such as 4, 5, 6, 7, 8, 9, 10, 20, 30, 40, 50, 60, 70, 80, 90, 100, etc. μg per dose. Administration of the CD81-binding peptide of the present invention can elicit an anti-E2 antibody titer in the treated mammal that lasts for at least 1 week, 2 weeks, 1 month, 2 months, 3 months, 4 months, 5 months, 6 months, a year, or longer. CD81-binding peptides of HCV E2 can also be administered to provide a memory immune response. If such response is achieved, antibody titers may decline over time, however, exposure to the HCV virus immunogen results in the rapid induction of antibodies, e.g., within only a few days. Optionally, antibody titers can be maintained in a mammal by providing one or more booster injections of the CD81-binding peptide of HCV E2 according to the present invention at 2 weeks, 1 month, 2 months, 3 months, 4 months, 5 months, 6 months, 1 year, or more after the primary injection.

### Example

The Example is designed in order to further illustrate the present invention and serves a better understanding. It is not to be construed as limiting the scope of the invention in any way.

The neutralizing properties of sera induced by immunization with Ad6E2 (Adenovirus vector encoding E2 of HCV genotype 1b, isolate T212 corresponding to the amino acid sequence set forth in SEQ ID NO: 3) or Ad6DeltaE2 (Adenovirus vector encoding E2 of HCV genotype 1b, isolate T212 lacking HVR1 corresponding to amino acids 28 to 364 of the amino acid sequence set forth in SEQ ID NO: 3) were tested in a model system in which cell culture derived HCV (HCVcc) was used to infect cultured Huh7.5 human hepatoma cells (Lindenbach et al., 2005; Wakita et al., 2005; Zhong et al., 2005).

Infectivity was measured by testing the HCV RNA by quantitative PCR analysis from infected Huh7.5 cells. Three HCVcc displaying HCV envelope from the 1a (H77), 1b (ukn), 2a (J6) genotypes were used in these assays to assess the cross-neutralization potential of the induced antibodies.

Antisera from immunized mice were heat-inactivated at 56° C. for 1 h, and then mixed with HCVcc in 10% FBS/DMEM at the dilution of 1:300 (for sera of Ad6E2 immunized mice) or 1:100 (for sera of Ad6DeltaE2 immunized mice) and incubated at 37° C. for 1 h. The virus/serum mixture was transferred to Huh7.5 cells seeded in 12 well plates (8×10⁴ cells per well) and incubated at 37° C. for 6 h. The virus-containing media was removed, replaced with DMEM with 10% FBS and incubated at 37° C. HCVcc infections were terminated after 72 h by cell lysis and infectivity was measure by quantitative PCR on viral RNA. Neutralization was determined by comparing viral infectivity in the presence of immune sera to the mean infectivity in the presence of pre-immune control samples at the same dilution.

For generation of HCVcc, J6/JFH1, J6/JFH1/E1E2 ukn and J6/JFH1/EIE2 H77 RNAs were synthesized by in vitro transcription of an XbaI-linearized template using the T7 MEGAscript kit (Ambion) and purification with the RNeasy mini kit (QIAGEN) with on-column DNase treatment as already described. RNAs were transfected to Huh7.5 cells by the TransIT-mRNA Transfection kit (Mints) according to the manufacturer protocol. Supernatants from transfected cells containing viruses were harvested 3-4 days post transfection, cleared through a 0.45 μm pore size filters and used either directly for infections or stored at 4° C. for 2-3 weeks without loosing significant amount of infectivity.

For quantification analysis of HCV RNA from infected cells, RNA was prepared by using “RNeasy mini kit” columns (Qiagen), and eluted in a volume of 50 μl RNase-free water. 1-10 microliters of the respective sample was used for quantitative RT-PCR analysis employing an ABI PRISM 7900HT Sequence Detector (Applied Biosystems, Darmstadt, Germany). Amplifications were conducted at least in duplicate with the Taqman 2X Universal PCR Master Mix No AmpErase UNG (Applied Biosystems) using the following primers and 3′-phosphate-blocked, 6-carboxyfluorescine (6-FAM)- and tetrachloro-6-carboxyfluorescine (TAMRA)-labeled probes (Applied Biosystems): HCV-JFH1 Taqman probe, 5′-6FAM-AAA GGA CCC AGT CTT CCC GGC AA-TAMRA-3′ (SEQ ID NO: 24); HCV-JFH1-S147, 5′-TCT GCG GAA CCG GTG AGT A-3′ (SEQ ID NO: 25); HCV-JFH1-A221, 5′-GGG CAT AGA GTG GGT TTA TCC A-3′ (SEQ ID NO: 26).

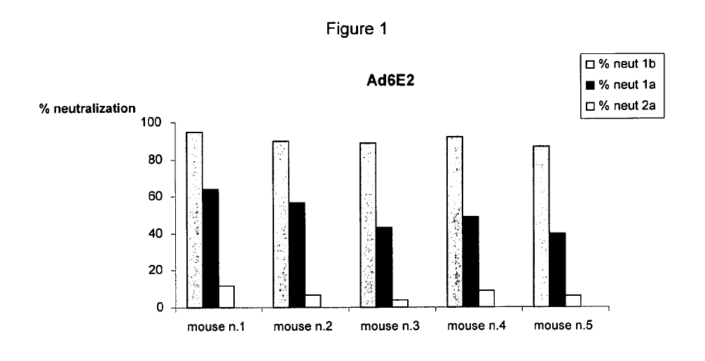

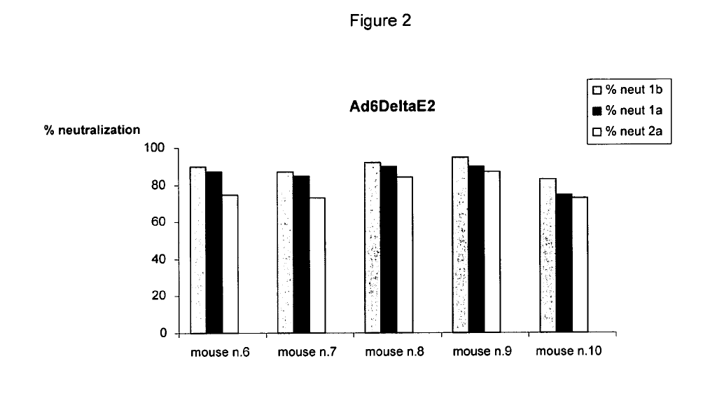

Sera from animals immunized with full length E2 expressing vector (Ad6E2) displayed higher neutralization activity on the homologous HCVcc with respect to immune sera from Ad6DeltaE2 immunized mice. This finding is consistent with the lack of anti-HVR1 neutralizing antibodies in the latter antiserum. However, antibodies induced by the E2 vector showed limited ability to interfere with the infection from the heterologous HCVcc (FIG. 1). In contrast, mice immunized with the DeltaE2 Adenovirus vector were capable of neutralizing all three tested HCVcc (FIG. 2).

### REFERENCES CITED

Baron, U. et al., (1997), Nucleic Acids Res. 25:2723-2729
Chapman, B. S. et al., (1991), Nucleic Acids Res. 19:3979-3986
Combet, C. et al., (2007), Nucleic Acids Res. 35:D363-D366
Dubuisson, J. et al., (2008), Cellular Microbiology 10:821-827
Facciabene, A. et al., (2007), Vaccine 26:47-58
Flint, M. et al., (1999), J. Virol. 73:6235-6244
Hagstrom, J. N. et al., (2000), Blood 95:2536-2542
Higginbottom, A. et al., (2000), J. Virol. 74:3642-3649
Keck, Z. Y. et al., (2008), Curr. Top. Micobiol. Immunol. 317:1-38
Koutsoudakis, G. et al., (2006), J. Virol. 80(11):5308-5320
Kuiken, C. et al., (2005), Bioinformatics 21:379-384
Lasaro, M. O. et al., (2008), Nat. Med. 14:205-12
Lavie, M. et al., (2007), Curr. Issues Mol. Biol. 9:71-86
Leuenberger, H. G. W. et al., Eds., (1995), Helvetica Chimica Acta, CH-4010 Basel, Switzerland
Li, X. et al., (1999), Nat. Biotechnol. 17:241-245
Lindenbach, B. D. et al, (2005), Science 309:623-626
Makarov, S. S. et al., (1994), Nucleic Acids Res. 22:1504-1505
Meola, A. et al., (2000), J. Virol. 74:5933-5938
Newman, M. J. et al., (1998), Critical Reviews in Therapeutic Drug Target Carrier Systems 15:89-142
Robert-Guroff, M., (2007), Curr. Opin. Biotechnol. 18:546-556
Roccasecca, R. et al., (2003), J Virol. 77:1856-67
Simmonds, P., (2004), J. Gen. Virol. 85:3173-3188
Timm, J. and Roggendorf, M., (2007), World J. Gastroenterol. 13:4808-4817
Wakita, T. et al., (2005), Nature Med. 11:791-796
Xu, Z. L., (2001), Gene 272:149-156
Zhong, J. et al., (2005), Proc. Natl. Acad. Sci. USA 102:9294-9299

We claim:

1. A CD81-binding peptide of HCV E2, which is devoid of or mutated within the N-terminal 27 amino acids of the mature E2 envelope glycoprotein, or variant thereof which retains the ability to bind to CD81,
a polypeptide comprising said peptide, with the proviso that said polypeptide is not a wild type E2,
a polynucleotide encoding said peptide or said polypeptide,
an expression cassette comprising (i) said polynucleotide, and (ii) one or more polynucleotides selected from the group consisting of poly-adenylation signal, promoter, enhancer, a nucleotide sequence encoding a heterologous protein, and a nucleotide sequence encoding a peptide-tag,
a vector comprising said polynucleotide or said expression cassette,
a composition comprising said peptide, said polypeptide, said polynucleotide, said expression cassette, or said vector, and an adjuvant, or
a pharmaceutical composition comprising said peptide, said polypeptide, said polynucleotide, said expression cassette, said vector, or said composition, and a pharmaceutically acceptable excipient, carrier, or diluent,
for induction of an immune response against HCV in a mammal.

2. The CD81-binding peptide, the polypeptide, the polynucleotide, the expression cassette, the vector, the composition, or the pharmaceutical composition according to claim 1, wherein the immune response is therapeutic and/or prophylactic.

3. The CD81-binding peptide, the polypeptide, the polynucleotide, the expression cassette, the vector, the composition, or the pharmaceutical composition according to claim 1 wherein the immune response is directed against two or more different HCV genotypes.

4. The CD81-binding peptide according to claim 1 wherein N amino acids of the HVR1 are mutated or deleted, wherein N is any number from 1 to 27.

5. The CD81-binding peptide according to claim 1 which exhibits increased CD81 binding when compared to wild type E2 glycoproteins having a wild type HVR1.

6. The CD81-binding peptide according to claim 1 which is derived from a naturally occurring HCV genotype.

7. The CD81-binding peptide according to claim 1 wherein the HCV genotype is selected from the group consisting of: 1, 1a, 1b, 1c, 2, 2a, 2b, 2c, 3, 3a, 3b, 4, 4a, 4b, 4c, 4d, 4e, 5, 5a, 6, 6a, 7, 7a, 7b, 8, 8a, 8b, 9, 9a, 10, 10a, 11, and 11a.

8. The CD81-binding peptide according to claim 1 which corresponds to amino acids 28 to 364 of the amino acid sequence set forth in SEQ ID NO: 3 or 9 or variants thereof, or to amino acids 28 to 363 of the amino acid sequence set forth in SEQ ID NO: 5 or 7 or variants thereof.

9. The CD81-binding peptide according to claim 1 which is selected from the group consisting of amino acid sequences starting at amino acid position 28 and ending at amino acid position 363, 361, 334, 333, 332, 331, 302, 301, 300, 299, 283, 282, 281, 280, 279, 278, or 277 of the amino acid sequence set forth in SEQ ID NO: 3 or 9, or position 362, 361, 333, 332, 331, 330, 301, 300, 299, 298, 282, 281, 280, 279, 278, 277, or 276 of the amino acid sequence set forth in SEQ ID NO: 5 or 7.

10. The polypeptide according to claim 1 further comprising the HCV envelope glycoprotein E1 or a fragment thereof.

11. The polypeptide according to claim 1 wherein the CD81-binding peptide sequence is preceded by the carboxy terminal 14 amino acids of E1, preferably having an amino acid sequence as set forth in SEQ ID NO: 11, 12, or 13.

12. The polypeptide according to claim 1 wherein the CD81-binding peptide sequence is preceded by the tissue plasminogen activator signal sequence, preferably having an amino acid sequence as set forth in SEQ ID NO: 15.

13. The vector according to claim 1 wherein the vector is selected from the group consisting of a plasmid DNA vector, a viral vector, a viral-like particle, a bacterial spore, and a bacteriophage.

14. The vector according to claim 13, which is a plasmid DNA, an adenovirus (Ad) vector (e.g., a non-replicating Ad5, Ad11, Ad26, Ad35, Ad49, ChAd3, ChAd4, ChAd5, ChAd6, ChAd7, ChAd8, ChAd9, ChAd10, ChAd11, ChAd16, ChAd17, ChAd19, ChAd20, ChAd22, ChAd24, ChAd26, ChAd30, ChAd31, ChAd37, ChAd38, ChAd44, ChAd63, ChAd82, ChAd55, ChAd73, ChAd83, ChAd146, ChAd147, PanAd1, PanAd2, and PanAd3 vector or a replication-competent Ad4 and Ad7 vector), an adeno-associated virus (AAV) vector (e.g., an MV type 5), an alphavirus vector (e.g., Venezuelan equine encephalitis virus (VEE), sindbis virus (SIN), semliki forest virus (SFV), and VEE-SIN chimeras), a herpes virus vector, a measles virus vector, a pox virus vector (e.g., vaccinia virus, modified vaccinia virus Ankara (MVA), NYVAC (derived from the Copenhagen strain of vaccinia), and an avipox vector such as a canarypox (ALVAC) and fowlpox virus (FPV) vector), or a vesicular stomatitis virus vector.

15. The composition according to claim 1 wherein the adjuvant is an agonist for a receptor selected from the group consisting of type I cytokine receptors, type II cytokine receptors, TNF receptors, vitamin D receptor acting as transcription factor, and the Toll-like receptors 1 (TLR1), TLR-2, TLR 3, TLR4, TLR5, TLR-6, TLR7, and TLR9.

16. The composition according to claim 15, wherein the adjuvant is a Toll-like receptor 4 or 9 agonist.

17. Method for inducing an immune response in a mammal against HCV comprising administering to said mammal
a CD81-binding peptide of HCV E2, which is devoid of or mutated within the N-terminal 27 amino acids of the mature E2 envelope glycoprotein, or variant thereof which retains the ability to bind to CD81,
a polypeptide comprising said peptide, with the proviso that said polypeptide is not a wild type E2,
a polynucleotide encoding said peptide or said polypeptide,
an expression cassette comprising (i) said polynucleotide, and (ii) one or more polynucleotides selected from the group consisting of poly-adenylation signal, promoter, enhancer, a nucleotide sequence encoding a heterologous protein, and a nucleotide sequence encoding a peptide-tag,
a vector comprising said polynucleotide or said expression cassette,
a composition comprising said peptide, said polypeptide, said polynucleotide, said expression cassette, or said vector, and an adjuvant, or
a pharmaceutical composition comprising said peptide, said polypeptide, said polynucleotide, said expression cassette, said vector, or said composition, and a pharmaceutically acceptable excipient, carrier, or diluent,
in an amount effective to generate an immune response.

18. The method according to claim 17, wherein the immune response is therapeutic and/or prophylactic.

19. The method according to claim 17 wherein the immune response is directed against two or more different HCV genotypes.

20. The method according to claim 17 wherein at least one booster dose comprising the CD81-binding peptide, the polypeptide, the polynucleotide, the expression cassette, the vector, the composition, or the pharmaceutical composition is administered to the mammal in an amount effective to enhance the immune response.

21. The method according to claim 20, wherein the vector for generating the immune response and the vector for enhancing the immune response are the same.

22. The method according to claim 20, wherein the vector for generating the immune response and the vector for enhancing the immune response are different.

23. The method according to claim 20 wherein the vector for generating the immune response is selected from the group consisting of DNA plasmid, adenovirus (Ad) vectors (e.g., non-replicating Ad5, Ad11, Ad26, Ad35, Ad49, ChAd3, ChAd4, ChAd5, ChAd6, ChAd7, ChAd8, ChAd9, ChAd10, ChAd11, ChAd16, ChAd17, ChAd19, ChAd20, ChAd22, ChAd24, ChAd26, ChAd30, ChAd31, ChAd37, ChAd38, ChAd44, ChAd63, ChAd82, ChAd55, ChAd73, ChAd83, ChAd146, ChAd147, PanAd1, PanAd2, and PanAd3 vectors or replication-competent Ad4 and Ad7 vectors), adeno-associated virus (AAV) vectors (e.g., MV type 5), alphavirus vectors (e.g., Venezuelan equine encephalitis virus (VEE), sindbis virus (SIN), semliki forest virus (SFV), and VEE-SIN chimeras), herpes virus vectors, measles virus vectors, pox virus vectors (e.g., vaccinia virus, modified vaccinia virus Ankara (MVA), NWAC (derived from the Copenhagen strain of vaccinia), and avipox vectors: canarypox (ALVAC) and fowlpox (FPV) vectors), and vesicular stomatitis virus vectors, and the vector for enhancing the immune response is selected from the group consisting of DNA plasmid, adenovirus (Ad) vectors (e.g., non-replicating Ad5, Ad11, Ad26, Ad35, Ad49, ChAd3, ChAd4, ChAd5, ChAd6, ChAd7, ChAd8, ChAd9, ChAd10, ChAd11, ChAd16, ChAd17, ChAd19, ChAd20, ChAd22, ChAd24, ChAd26, ChAd30, ChAd31, ChAd37, ChAd38, ChAd44, ChAd63, ChAd82, ChAd55, ChAd73, ChAd83, ChAd146, ChAd147 PanAd1, PanAd2, and PanAd3 vectors or replication-competent Ad4 and Ad7 vectors), adeno-associated virus (MV) vectors (e.g., AAV type 5), alphavirus vectors (e.g., Venezuelan equine encephalitis virus (VEE), sindbis virus (SIN), semliki forest virus (SFV), and VEE-SIN chimeras), herpes virus vectors, measles virus vectors, pox virus vectors (e.g., vaccinia virus, modified vaccinia virus Ankara (MVA), NWAC (derived from the Copenhagen strain of vaccinia), and avipox vectors: canarypox (ALVAC) and fowlpox (FPV) vectors), and vesicular stomatitis virus vectors.

24. The method according to claim 23 with the proviso that the vector for generating the immune response and the vector for enhancing the immune response are different.

25. The method according to claim 24, wherein the vector for generating the immune response and the vector for enhancing the immune response are serologically different, non-cross-reacting adenovirus vectors.

26. A CD81-binding peptide, which corresponds to amino acids 28 to 364 of the amino acid sequence set forth in SEQ ID NO: 3 or 9 or variants thereof, or to amino acids 28 to 363 of the amino acid sequence set forth in SEQ ID NO: 5 or 7 or variants thereof.

27. The CD81-binding peptide according to claim 26, which is selected from the group consisting of amino acid sequences starting at amino acid position 28 and ending at amino acid position 363, 361, 334, 333, 332, 331, 302, 301, 300, 299, 283, 282, 281, or 280 of the amino acid sequence set forth in SEQ ID NO: 3 or 9, or position 362, 361, 333, 332, 331, 330, 301, 300, 299, 298, 282, 281, 280, or 279 of the amino acid sequence set forth in SEQ ID NO: 5 or 7.

28. A polypeptide comprising the CD81-binding peptide according to claim 26 with the proviso that said polypeptide is not a wild type E2.

29. The polypeptide according to claim 28 further comprising the HCV envelope glycoprotein E1 or a fragment thereof.

30. The polypeptide according to claim 28 wherein the CD81-binding peptide sequence is preceded by the carboxy terminal 14 amino acids of E1, preferably having an amino acid sequence as set forth in SEQ ID NO: 11, 12, or 13.

31. The polypeptide according to claim 28, wherein the CD81-binding peptide sequence is preceded by the tissue plasminogen activator signal sequence, preferably having an amino acid sequence as set forth in SEQ ID NO: 15.

32. A polynucleotide encoding the CD81-binding peptide according to claim 26.

33. An expression cassette comprising (i) the polynucleotide of claim 32, and (ii) one or more polynucleotides selected from the group consisting of poly-adenylation signal, promoter, enhancer, a nucleotide sequence encoding a heterologous protein, and a nucleotide sequence encoding a peptide-tag.

34. A vector comprising a polynucleotide according to claim 32 or a polynucleotide encoding a CD81-binding peptide of claim 1.

35. The vector according to claim 34, wherein the vector is selected from the group consisting of a plasmid DNA vector, a viral vector, a viral-like particle, a bacterial spore, and a bacteriophage.

36. The vector according to claim 35, which is a plasmid DNA, an adenovirus (Ad) vector (e.g., a non-replicating Ad5, Ad11, Ad26, Ad35, Ad49, ChAd3, ChAd4, ChAd5, ChAd6, ChAd7, ChAd8, ChAd9, ChAd10, ChAd11, ChAd16, ChAd17, ChAd19, ChAd20, ChAd22, ChAd24, ChAd26, ChAd30, ChAd31, ChAd37, ChAd38, ChAd44, ChAd63, ChAd82, ChAd55, ChAd73, ChAd83, ChAd146, ChAd147 PanAd1, PanAd2, and PanAd3 vector or a replication-competent Ad4 and Ad7 vector), an adeno-associated virus (AAV) vector (e.g., an AAV type 5), an alphavirus vector (e.g., Venezuelan equine encephalitis virus (VEE), sindbis virus (SIN), semliki forest virus (SFV), and VEE-SIN chimeras), a herpes virus vector, a measles virus vector, a pox virus vector (e.g., vaccinia virus, modified vaccinia virus Ankara (MVA), NYVAC (derived from the Copenhagen strain of vaccinia), and an avipox vector such as a canarypox (ALVAC) and fowlpox virus (FPV) vector), or a vesicular stomatitis virus vector.

37. A composition comprising a CD81-binding peptide according to claim 26, a polypeptide according to claim 28 a polynucleotide according to claim 32, an expression cassette according claim 33 and an adjuvant.

38. The composition according to claim 37, wherein the adjuvant is an agonist for a receptor selected from the group consisting of type I cytokine receptors, type II cytokine receptors, TNF receptors, vitamin D receptor acting as transcription factor, and the Toll-like receptors 1 (TLR1), TLR-2, TLR 3, TLR4, TLR5, TLR-6, TLR7, and TLR9.

39. The composition according to claim 38, wherein the adjuvant is a Toll-like receptor 4 or 9 agonist.

40. A pharmaceutical composition comprising a CD81-binding peptide according to claim 26 a polypeptide according to claim 28, a polynucleotide according to claim 32, an expression cassette according to claim 33, and a pharmaceutically acceptable excipient, carrier, or diluent.
